(19) United States
(12) Patent Application Publication
(10) Pub. No.: US 2010/0172983 A1
(43) Pub. Date: Jul. 8, 2010

(54) Pharmaceutical Compositions for the Coordinated Delivery of Naproxen and Esomeprazole

(76) Inventor: John R. Plachetka, Chapel Hill, NC (US)

Correspondence Address: LAW OFFICE OF MICHAEL A. SANZO, LLC, 15400 CALHOUN DR., SUITE 125, ROCKVILLE, MD, 20855, US

(21) Appl. No.: 12/553,804

(22) Filed: Sep. 3, 2009

Related U.S. Application Data
(63) Continuation-in-part of application No. 11/129,320, filed on May 16, 2005.
Application No. 11/129,320 is a continuation-in-part of application No. 10/158,216, filed on May 31, 2002, now Pat. No. 6,926,907.
(60) Provisional application No. 60/294,588, filed on Jun. 1, 2001.
(60) Provisional application No. 61/095,584, filed on Sep. 9, 2008.

(51) Int. Cl. A61K 9/24; A61K 31/4439; A61P 29/00; A61P 19/02

(52) U.S. Cl. 424/471; 514/338; 424/472

(57) ABSTRACT

The present disclosure is directed to drug dosage forms that release an agent that raises the pH of a patient's gastrointestinal tract, followed by a non-steroidal anti-inflammatory drug. The dosage form is designed so that the NSAID is not released until the intragastric pH has been raised to a safe level. The disclosure also encompasses methods of treating patients by administering this coordinated release, gastroprotective, antiarthritic/analgesic combination unit dosage form to achieve pain and symptom relief with a reduced risk of developing gastrointestinal damage such as ulcers, erosions and hemorrhages.

### CROSS REFERENCE TO RELATED APPLICATIONS

The present application claims the benefit of U.S. provisional application no. 61/095,584, filed on Sep. 9, 2008, and is a continuation-in-part of U.S. application Ser. No. 11/129,320, filed on May 16, 2005, which is a continuation-in-part of U.S. application Ser. No. 10/158,216, filed on May 31, 2002 (now patented as U.S. Pat. No. 6,926,907). The '216 application claims the benefit of U.S. provisional application No. 60/294,588, filed on Jun. 1, 2001. Each of the foregoing applications is incorporated herein by reference in its entirety.

### FIELD OF THE INVENTION

The present invention is directed to pharmaceutical compositions that provide for the coordinated release of an acid inhibitor and a non-steroidal anti-inflammatory drug (NSAID). These compositions have a reduced likelihood of causing unwanted side effects, especially gastrointestinal side effects, when administered as a treatment for pain, arthritis and other conditions amenable to treatment with NSAIDs. The present disclosure is further directed to a method for delivering a pharmaceutical composition to a patient in need thereof, comprising: administering to said patient a pharmaceutical composition in unit dose form comprising naproxen, or a pharmaceutically acceptable salt thereof, and esomeprazole, or a pharmaceutically acceptable salt thereof.

### BACKGROUND OF THE INVENTION

Over 15 million Americans take nonsteroidal anti-inflammatory drugs (NSAIDs) each day as a treatment for pain or inflammation. Although non-steroidal anti-inflammatory drugs are widely accepted as effective agents for controlling pain, their administration can lead to the development of gastroduodenal lesions, e.g., ulcers and erosions, in susceptible individuals. It appears that a major factor contributing to the development of these lesions is the presence of acid in the stomach and upper small intestine of patients. This view is supported by clinical studies demonstrating an improvement in NSAID tolerability when patients are also taking independent doses of acid inhibitors (Dig. Dis. 12:210-222 (1994); Drug Safety 21:503-512 (1999); Aliment. Pharmacol. Ther. 12:135-140 (1998); Am. J. Med. 104(3A):675-745 (1998); Clin. Ther. 17:1159-1173 (1995)). Other major factors contributing to NSAID-associated gastropathy include a local toxic effect of NSAIDs and inhibition of protective prostaglandins (Can. J. Gastroenterol. 13:135-142 (1999) and Pract. Drug Safety 21:503-512, (1999)), which may also make some patients more susceptible to the ulcerogenic effects of other noxious stimuli.

In general, more potent and longer lasting acid inhibitors, such as proton pump inhibitors, are thought to be more protective during chronic administration of NSAIDs than shorter acting agents, e.g., histamine H-2 receptor antagonists (H-2 blockers) (N. Eng. J. Med. 338:719-726 (1998); Am. J. Med. 104(3A):565-615 (1998)). The most likely explanation for this is that gastric pH fluctuates widely throughout the dosing interval with short acting acid inhibitors leaving the mucosa vulnerable for significant periods of time. In particular, the pH is at its lowest point, and hence the mucosa is most vulnerable, at the end of the dosing interval (least amount of acid inhibition) and for some time after the subsequent dose of acid inhibitor. In general, it appears that when a short acting acid inhibitor and an NSAID are administered simultaneously, NSAID-related mucosal damage occurs before the pH of the gastrointestinal tract can be raised and after the acid inhibiting effect of the short acting acid inhibitor dissipates.

Although longer lasting agents, such as proton pump inhibitors (PPIs), usually maintain a consistently higher gastroduodenal pH throughout the day, their antisecretory effect may be delayed for several hours and may not take full effect for several days (Clip. Pharmacokinet. 20:38-49 (1991)). Their effect may be diminished toward the end of the usual dosing interval. Intragastric pH rises particularly slowly with the first dose in a course of treatment since this class of drugs is enteric coated to avoid destruction by stomach acid. As a result, absorption is delayed for several hours. Even then, some patients fail to respond consistently to drugs of this type and suffer from “acid breakthrough” which again leaves them vulnerable to NSAID-associated gastroduodenal damage (Aliment. Pharmacol. Ther. 14:709-714 (2000)). Despite a significant reduction in gastroduodenal lesions with the concomitant administration of a proton pump inhibitor during six months of NSAID therapy, up to 16% of patients still develop ulcers, indicating that there remains substantial room for improvement (N. Eng. J. Med. 338:727-734 (1998)). Thus, the addition of a pH sensitive enteric coating to an NSAID could provide additional protection against gastroduodenal damage not provided by the H2 blocker or PPI alone. In addition, although long acting acid inhibitors may reduce the risk of GI lesions in chronic NSAID users, there are questions about the safety of maintaining an abnormally elevated pH in a patient's GI tract for a prolonged period of time (Scand. J. Gastroenterol. Suppl. 178:85-92 (1990)).

During recent years, attempts have been made to reduce the gastrointestinal risk associated with taking NSAIDs by administering agents that inhibit stomach acid secretion, such as, for example, proton pump inhibitors with the NSAID. Such formulations may be effective in improving NSAID tolerability through dosages of esomeprazole and naproxen that produce the desired pharmacodynamic response and pharmacokinetic values. Parameters that may influence the desired pharmacodynamic response and pharmacokinetic values include, but are not limited to, for example, the dosage of each; extent of drug absorption; extent of drug distribution, and the duration of drug administration.

Recognizing the potential benefits of PPIs for the prevention of NSAID-induced gastroduodenal damage, others have disclosed strategies for combining the two active agents for therapeutic purposes. However, these suggestions do not provide for coordinated drug release or for reducing intragastric acid levels to a non-toxic level prior to the release of NSAID (U.S. Pat. No. 5,204,118; U.S. Pat. No. 5,417,980; U.S. Pat. No. 5,466,436; and U.S. Pat. No. 5,037,815). In certain cases, suggested means of delivery would expose the gastrointestinal tract to NSAIDs prior to onset of PPI activity (U.S. Pat. No. 6,365,184).

Attempts to develop NSAIDs that are inherently less toxic to the gastrointestinal tract have met with only limited success. For example, the recently developed cyclooxygenase-2 (COX-2) inhibitors show a reduced tendency to produce gastrointestinal ulcers and erosions, but a significant risk is still present, especially if the patient is exposed to other ulcerogens (JAMA 284:1247-1255 (2000); N. Eng. J. Med. 343:1520-1528 (2000)). In this regard, it appears that even low doses of aspirin will negate most of the benefit relating to lower gastrointestinal lesions. In addition, the COX-2 inhibitors may not be as effective as other NSAIDs at relieving some types of pain and have been associated with significant cardiovascular problems (JADA 131:1729-1737 (2000); SCRIP 2617, pg. 19, Feb. 14, 2001); NY Times, May 22, 2001, pg. Cl)).

Other attempts to produce an NSAID therapy with less gastrointestinal toxicity have involved the concomitant administration of a cytoprotective agent. In 1998, Searle began marketing Arthrotec™ for the treatment of arthritis in patients at risk for developing GI ulcers. This product contains misoprostol (a cytoprotective prostaglandin) and the NSAID diclofenac. Although patients administered Arthrotec™ do have a lower risk of developing ulcers, they may experience a number of other serious side effects such as diarrhea, severe cramping and, in the case of pregnant women, potential damage to the fetus.

Another approach has been to produce enteric coated NSAID products. However, even though these have shown modest reductions in gastroduodenal damage in short term studies (Scand. J. Gastroenterol. 20: 239-242 (1985) and Scand. J. Gastroenterol. 25:231-234 (1990)), there is no consistent evidence of a long term benefit during chronic treatment.

Overall, it may be concluded that the risk of inducing GI ulcers is a recognized problem associated with the administration of NSAIDs and that, despite considerable effort, an ideal solution has not yet been found. Furthermore, there is a need for a clinically effective therapy that delivers to a patient in need thereof a pharmaceutical composition in a unit dose form comprising naproxen, or pharmaceutically acceptable salt thereof, and esomeprazole, or pharmaceutically acceptable salt thereof, for a duration sufficient to achieve an instragastric pH of about 4 or greater and a plasma level of naproxen that is efficacious.

### SUMMARY OF THE INVENTION

The present invention is based upon the discovery of a new method for reducing the risk of gastrointestinal side effects in people taking NSAIDs for pain relief and for other conditions, particularly during chronic treatment. The method involves the administration of a single, coordinated, unit-dose product that combines: a) an agent that actively raises intragastric pH to levels associated with less risk of NSAID-induced ulcers; and b) an NSAID that is specially formulated to be released in a coordinated way that minimizes the adverse effects of the NSAID on the gastroduodenal mucosa. Either short or long acting acid inhibitors can be effectively used in the dosage forms. This method has the added benefit of being able to protect patients from other gastrointestinal ulcerogens whose effect may otherwise be enhanced by the disruption of gastroprotective prostaglandins due to NSAID therapy.

In its first aspect, the invention is directed to a pharmaceutical composition in unit dosage form suitable for oral administration to a patient. The composition contains an acid inhibitor present in an amount effective to raise the gastric pH of a patient to at least 3.5, preferably to at least 4, and more preferably to at least 5, when one or more unit dosage forms are administered. The gastric pH should not exceed 7.5 and preferably should not exceed 7.0. The term “acid inhibitor” refers to agents that inhibit gastric acid secretion and increase gastric pH. In contrast to art teaching against the use of H2 blockers for the prevention of NSAID-associated ulcers (N. Eng. J. Med. 340:1888-1899 (1999)), these agents are preferred compounds in the current invention. Specific H2 blockers that may be used include cimetidine, ranitidine, ebrotidine, pabutidine, lafutidine, loxtidine or famotidine. The most preferred acid inhibitor is famotidine present in dosage forms in an amount of between 5 mg and 100 mg.

Other preferred agents that may be effectively used as acid inhibitors are the proton pump inhibitors such as omeprazole, esomeprazole, pantoprazole, lansoprazole, rabeprazole, pariprazole, leminoprazole and tenatoprazole. Examples of particular proton pump inhibitors include omeprazole, present in unit dosage forms in an amount of between 5 mg and 50 mg; lansoprazole, present in unit dosage forms in an amount of between 5 mg and 150 mg (and preferably at between 5 mg and 30 mg); and pantoprazole, present in unit dosage forms in an amount of between 10 mg and 200 mg. Recently, a newer class of acid inhibitor has been developed which competes with potassium at the acid pump. The compounds in this class have been referred to as “reversible proton pump inhibitors” or “acid pump antagonists” and may also be used in the present invention. Examples include AZD-0865, AR-H047108, CS-526, pumaprazole, revaprazan and soraprazan (see WO9605177 and WO9605199). Other compounds in this group are H-335/25 (AstraZeneca, Dialog file 128, accession number 020806); Sch-28080 (Schering Plough, Dialog file 128, accession number 009663); Sch-32651 (Schering Plough, Dialog file 128, accession number 006883) and SK&F-96067 (CAS Registry no. 115607-61-9).

The pharmaceutical composition also contains a non-steroidal anti-inflammatory drug in an amount effective to reduce or eliminate pain or inflammation. The NSAID may be celecoxib, rofecoxib, lumiracoxib, valdecoxib, parecoxib, etoricoxib, CS-502, JTE-522, L-745,337, NS398, aspirin, acetaminophen (considered to be an NSAID for the purposes of the present invention), ibuprofen, flurbiprofen, ketoprofen, naproxen, oxaprozin, etodolac, indomethacin, ketorolac, lornoxicam, meloxicam, piroxicam, droxicam, tenoxicam, nabumetone, diclofenac, meclofenamate, mefenamic acid, diflunisal, sulindac, tolmetin, fenoprofen, suprofen, benoxaprofen, aceclofenac, tolfenamic acid, oxyphenbutazone, azapropazone, and phenylbutazone. The most preferred NSAID is naproxen in an amount of between 50 mg and 1500 mg, and more preferably, in an amount of between 200 mg and 600 mg. It will be understood that, for the purposes of the present invention, reference to an acid inhibitor, NSAID, or analgesic agent will include all of the common forms of these compounds and, in particular, their pharmaceutically acceptable salts. The amounts of NSAIDs which are therapeutically effective may be lower in the current invention than otherwise found in practice due to potential positive kinetic interaction and NSAID absorption in the presence of an acid inhibitor.

Preferably, the pharmaceutical composition of the present invention is in the form of a tablet or capsule that has: (a) the acid inhibitor present in an amount effective to raise the gastric pH of a patient to at least 3.5 upon the administration of one or more unit dosage forms; and (b) the non-steroidal anti-inflammatory drug (NSAID) present in an amount effective to reduce or eliminate pain or inflammation in a patient upon administration of one or more of said unit dosage forms. The NSAID in the dosage form should be in a core, preferably a single core when tablets are used, that is surrounded by a coating that does not release NSAID until the pH of the surrounding medium is 3.5 or higher. In the case of capsules, there may be several cores of NSAID, i.e., there may be multiple particles, each being surrounded by a coating that does not release NSAID until the pH of the surrounding medium is 3.5 or higher. The acid inhibitor is in one or more layers outside of the core which do not contain any NSAID. These layers are not surrounded by an enteric coating and, upon ingestion of the tablet or capsule by a patient, release the acid inhibitor into the patient's stomach.

The term “unit dosage form” as used herein refers to a single entity for drug administration. For example, a single tablet or capsule combining both an acid inhibitor and an NSAID would be a unit dosage form. A unit dosage form of the present invention preferably provides for coordinated drug release in a way that elevates gastric pH and reduces the deleterious effects of the NSAID on the gastroduodenal mucosa, i.e., the acid inhibitor is released first and the release of NSAID is delayed until after the pH in the GI tract has risen. For example, a single tablet or capsule containing both esomeprazole and naproxen is a unit dosage form. Unit dosage forms of the present disclosure provide for sequential drug release in a way that elevates gastric pH and reduces the deleterious effects of naproxen on the gastroduodenal mucosa, i.e., the esomeprazole is released first and the release of naproxen is delayed until after the pH in the GI tract has risen to 3.5 or greater. A “unit dosage form” (or “unit dose form”) may also be referred to as a “fixed dosage form” (or “fixed dose form”) or “fixed dosage combination” (or “fixed dose combination”) and are otherwise interchangeable.

In a preferred embodiment, the unit dosage form is a multilayer tablet, having an outer layer comprising the acid inhibitor and an inner core which comprises the NSAID. In the most preferred form, coordinated delivery is accomplished by having the inner core surrounded by a polymeric barrier coating that does not dissolve unless the surrounding medium is at a pH of at least 3.5, preferably at least 4 and more preferably, at least 5. Alternatively, a barrier coating may be employed which controls the release of NSAID by time, as opposed to pH, with the rate adjusted so that NSAID is not released until after the pH of the gastrointestinal tract has risen to at least 3.5, preferably at least 4, and more preferably at least 5. Thus, a time-release formulation may be used to prevent the gastric presence of NSAID until mucosal tissue is no longer exposed to the damage enhancing effect of very low pH.

One NSAID of special interest in dosage forms is aspirin which not only provides relief from pain and inflammation but may also be used in low doses by patients to reduce the risk of stroke, heart attack and other conditions. Thus, pharmaceutical compositions may contain an acid inhibitor in combination with aspirin in an amount effective, upon the administration of one or more unit dosage forms, to achieve any of these objectives. As with the compositions described above the unit dosage form can be a tablet or capsule in which aspirin is present in a core and is surrounded by a coating that does not release the aspirin until the pH of the surrounding medium is 3.5 or higher. The acid inhibitor is in one or more layers outside the core, which do not include an NSAID, are not surrounded by an enteric coating; and, upon ingestion of the dosage form by a patient, release the acid inhibitor into the patient's stomach. Any of the acid inhibitors described herein may be used in the aspirin-containing dosage forms. In dosage forms designed for providing low dose aspirin therapy to patients, the aspirin should typically be present at 20-200 mg.

An embodiment of the present disclosure is directed to a pharmaceutical composition in unit dosage form suitable for administration to a patient, comprising:
(a) esomeprazole, at least a portion of which is not surrounded by an enteric coating; and
(b) naproxen, wherein the naproxen is surrounded by a coating that substantially inhibits the release of the naproxen from the dosage form unless it is exposed to an environment with a pH of about 3.5 or higher;
wherein the unit dosage form provides for release of the esomeprazole and the naproxen such that:
   i) upon introduction of the unit dosage form into a medium, at least a portion of the esomeprazole is released regardless of the pH of the medium; and
   ii) the naproxen is released when the pH of the environment is 3.5 or higher.

In certain embodiments, the coating surrounding the naproxen in the pharmaceutical compositions in unit dosage form disclosed herein may dissolve when the pH of the environment is 4.0 or higher, 4.5 or higher, 5.0 or higher, or 5.5 or higher. In some embodiments, the naproxen may be present in the pharmaceutical compositions in unit dosage form in an amount of between 200 mg and 600 mg. In other embodiments, the esomeprazole may be present in the pharmaceutical compositions in unit dosage form in an amount of between 5 mg and 100 mg. In another embodiment, the unit dosage form is suitable for oral administration to a patient. In yet another embodiment, the unit dosage form is a tablet. In still another embodiment, the unit dosage form is a multilayer tablet comprising a single core and one or more layers outside of the core, wherein:
i) the naproxen is present in the core;
ii) the coating surrounds the core; and
iii) the esomeprazole is in the one or more layers outside the core.

In an embodiment of the present disclosure, the one or more layers outside of the core do not contain naproxen and are not surrounded by an enteric coating. In another embodiment, the unit dosage form is a bilayer tablet having an outer layer of the esomeprazole and an inner core of the naproxen and wherein the outer layer of the tablet is surrounded by a non-enteric film coating. In other embodiments, the coating surrounding the core substantially inhibits the release of the naproxen unless it is in a medium with a pH of 4.0 or greater, 4.5 or greater, 5.0 or greater, or 5.5 or greater.

Another embodiment of the present disclosure is directed to a method of treating pain or inflammation in a patient in need of such treatment, comprising:
(a) orally administering to the patient esomeprazole, wherein at least a portion of the esomeprazole is not surrounded by an enteric coating, wherein the dose of the esomeprazole is effective to raise the gastric pH of the patient to at least 3.5 or higher; and
(b) orally administering to the patient naproxen, wherein the naproxen is surrounded by a coating that substantially inhibits the release of the naproxen unless it is in an environment with a pH of 3.5 or higher,

wherein the naproxen is released and is effective to treat the pain or inflammation of the patient. In certain embodiments, and the pain or inflammation is due to osteoarthritis and/or rheumatoid arthritis.

Yet another embodiment of the present disclosure is directed to a method of treating ankylosing spondylitis in a patient in need of such treatment, comprising:
(a) orally administering to the patient esomeprazole, wherein at least a portion of the esomeprazole is not surrounded by an enteric coating, wherein the dose of the esomeprazole is effective to raise the gastric pH of the patient to at least 3.5 or higher; and
(b) orally administering to the patient naproxen, wherein the naproxen is surrounded by a coating that substantially inhibits the release of the naproxen unless it is in an environment with a pH of 3.5 or higher,

wherein the naproxen is released and is effective to treat the ankylosing spondylitis of the patient.

In certain embodiments disclosed herein, the coating surrounding the naproxen substantially inhibits the release of the naproxen unless it is in an environment with a pH of 4.0 or higher, 4.5 or higher, 5.0 or higher, or 5.5 or higher. In some embodiments, the naproxen may be present in an amount of between 200 mg and 600 mg. In other embodiments, the esomeprazole may be present in an amount of between 5 mg and 100 mg. In still other embodiments, the esomeprazole and the naproxen are delivered as a unit dosage form. In some embodiments, the unit dosage form is suitable for oral administration to a patient. In another embodiment, the unit dosage form provides for the sequential release of the esomeprazole followed by the naproxen. In yet another embodiment, the unit dosage form is a tablet. In still another embodiment, the unit dosage form is a bilayer tablet having an outer layer of the esomeprazole and an inner core of the naproxen and wherein the outer layer of the tablet is surrounded by a non-enteric film coating.

In another aspect, the disclosure is directed to the use of naproxen, or pharmaceutically acceptable salt thereof, and esomeprazole, or pharmaceutically acceptable salt thereof in the manufacture of a pharmaceutical composition in unit dose form wherein said esomeprazole, or pharmaceutically acceptable salt thereof, is released from said unit dose form at a pH of from about 0 or greater to target: a mean % time at which intragastric pH remains at about 4.0 or greater for about a 24 hour period of at least about 41%. In another embodiment, the mean % time at which intragastric pH remains at about 4.0 or greater for about a 24 hour period is at least about 60%, at least about 71%, or at least about 77%. In another embodiment, the pharmaceutical composition further targets a pharmacokinetic (pk) profile having a mean maximum plasma concentration (Cₘₐₓ) for naproxen of about 76 μg/ml or about 79 μg/ml.

In another embodiment, the pharmaceutical composition targets a pharmacokinetic (pk) profile having a mean area under the plasma concentration-time curve from time zero when first dose is administered to about 24 hours after the first dose is administered (AUC₀₋₂₄) for esomeprazole of about 1500 hr*μg/mL, of about 2134 hr*μg/mL or about 4911 hr*μg/mL.

In one aspect, the disclosure is directed to the use of a naproxen, or pharmaceutically acceptable salt thereof, and esomeprazole, or pharmaceutically acceptable salt thereof in the manufacture of a pharmaceutical composition in unit dose form for delivery to a patient in need thereof, wherein a first unit dose form is administered as an AM dose and a second unit dose form is administered about 10 hours later as a PM dose to target:
i) a pharmacokinetic (pk) profile for naproxen where:
   a) the AM dose has a mean Cₘₐₓ of about 81 μg/mL and a median time to maximum concentration (Tₘₐₓ) of from about 2.5 to about 4 hours, and
   b) the PM dose has a mean Cₘₐₓ of about 76.2 μg/mL and a median Tₘₐₓ of from about 10 to about 14 hours; and
ii) a pharmacokinetic (pk) profile for esomeprazole where:
   a) the AM dose has a mean area under the plasma concentration-time curve from time zero when the AM dose is administered to about 10 hours after the AM dose is administered (AUC_{0-10,am}) of about 850 hr*μg/mL, and
   b) the PM dose has a mean area under the plasma concentration-time curve from time zero when the PM dose is administered to about 14 hours after the PM dose is administered (AUC_{0-14,pm}) of about 650 hr*μg/mL.

In one embodiment, the pharmaceutical composition provides for:
i) the mean Cₘₐₓ for said AM dose of naproxen is about 86.2 μg/mL or about 80.9 μg/mL and said median Tₘₐₓ is about 3.0 hours; and
(ii) the mean Cₘₐₓ for said PM dose is about 76.8 μg/mL or about 76.2 μg/mL and said median Tₘₐₓ is about 10 hours.

In another embodiment, the pharmaceutical composition described above provides for:
i) the mean area under the plasma concentration-time curve from time zero when the AM dose is administered to about 10 hours after the AM dose is administered (AUC_{0-10,am}) for said AM dose of esomeprazole is about 1216 hr*μg/mL or about 2779 hr*g/mL, and ii) the mean area under the plasma concentration-time curve from time zero when the PM dose is administered to about 14 hours after the PM dose is administered (AUC_{0-14,pm}) for said PM dose of esomeprazole is about 919 hr*μg/mL or about 2066 hr*μg/mL.

In another embodiment, the pharmaceutical composition targets a pharmacokinetic (pk) profile having a mean area under the plasma concentration-time curve from time zero when first dose is administered to about 24 hours after the first dose is administered (AUC₀₋₂₄) for esomeprazole is about 1500 hr*μg/mL, about 2000 hr*μg/mL, or about 4911 hr*μg/mL.

Another embodiment is directed to the use wherein the pharmaceutical composition further targets a mean % time at which intragastric pH remains at about 4.0 or greater for about a 24 hour period after reaching steady state of at least about 41%, about 60%, about 71%, or about 77%.

In one embodiment, the dose form is a multilayer tablet comprising at least one core and at least a first layer and a second layer, wherein:
i) said core comprises naproxen, or pharmaceutically acceptable salt thereof;
ii) said first layer is a coating that at least begins to release the naproxen, or pharmaceutically acceptable salt thereof, when the pH of the surrounding medium is about 3.5 or greater; and
iii) said second layer comprises esomeprazole, wherein said esomeprazole is released at a pH of from about 0 or greater.

In another embodiment, the dose form provides for the release of esomeprazole at a pH of from about 1 or greater, a pH of from about 0 to about 2, or at a pH of about 2 to 4. In another embodiment, at least a portion of the esomeprazole, or pharmaceutically acceptable salt thereof, is not coated with an enteric coating. In another embodiment, the first layer is an enteric coating. In yet another embodiment, the multi-layer tablet is substantially free of sodium bicarbonate. In one embodiment, the first layer begins to release the naproxen when the pH of the surrounding medium is at about 4.0 or about 4.5 or greater.

In another embodiment, the pharmaceutical composition in unit dose form comprises about 500 mg of said naproxen, or pharmaceutically acceptable salt thereof, and about 20 mg or about 30 mg, of said esomeprazole, or pharmaceutically acceptable salt thereof. In another embodiment, the pharmaceutical composition is administered twice a day for at least about 6 days or twice a day for at least 9 days.

In another embodiment, the pharmaceutical composition is for administration to an at risk patient. The at risk patient can be a patient being treated for a disease or disorder selected from pain and inflammation, or can be a patient being treated for osteoarthritis, rheumatoid arthritis, ankylosing spondylitis, or a combination thereof.

In one aspect, the disclosure is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater to target: a mean % time at which intragastric pH remains at about 4.0 or greater for about a 24 hour period after reaching steady state of at least about 41%.

In another aspect, the disclosure is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of about 81 μg/mL and a median time to maximum concentration (Tₘₐₓ) of from about 2.5 to about 4 hours, and
   b) the PM dose has a mean Cₘₐₓ of about 76.2 μg/mL and a median Tₘₐₓ of from about 10 to about 14 hours; and
ii) a pk profile for esomeprazole where:
   a) the AM dose has a mean area under the plasma concentration-time curve from time zero when the AM dose is administered to about 10 hours after the AM dose is administered (AUC_{0-10,am}) is about 850 hr*m/mL, and
   b) the PM dose has a mean area under the plasma concentration-time curve from time zero when the PM dose is administered to about 14 hours after the PM dose is administered (AUC_{0-14,pm}) is about 650 hr*m/mL.

Yet another aspect is directed to delivering a pharmaceutical composition in unit dose form that provides the pharmacodynamic response and/or pharmacokinetic values disclosed herein to a patient being treated for a disease or disorder selected from pain and inflammation. A further aspect is directed to delivering a pharmaceutical composition in unit dose form that provides the pharmacodynamic response and/or pharmacokinetic values disclosed herein to a patient being treated for osteoarthritis, rheumatoid arthritis, ankylosing spondylitis, or a combination thereof.

A still further aspect is directed to delivering a pharmaceutical composition in unit dose form that provides the pharmacodynamic response and/or pharmacokinetic values disclosed herein to an at risk patient. Another aspect is directed to delivering a pharmaceutical composition in unit dose form that provides the pharmacodynamic response and/or pharmacokinetic values disclosed herein to an at risk patient being treated for a disease or disorder selected from pain and inflammation. A further aspect is directed to delivering a pharmaceutical composition in unit dose form that provides the pharmacodynamic response and/or pharmacokinetic values disclosed herein to an at risk patient being treated for osteoarthritis, rheumatoid arthritis, ankylosing spondylitis, or a combination thereof.

Yet another aspect is directed to delivering a pharmaceutical composition in unit dosage form that provides the pharmacodynamic response and/or pharmacokinetic values disclosed herein via a multilayer tablet comprising at least one core and at least a first layer and a second layer, wherein:
i) said core comprises naproxen, or pharmaceutically acceptable salt thereof;
ii) said first layer is a coating that at least begins to release the naproxen, or pharmaceutically acceptable salt thereof, when the pH of the surrounding medium is at about 3.5 or greater; and said second layer is esomeprazole, or pharmaceutically acceptable salt thereof, wherein said esomeprazole, or pharmaceutically acceptable salt thereof, is released at a pH of from about 0 or greater.

The invention includes methods of treating a patient for pain, inflammation and/or other conditions by administering the pharmaceutical compositions described above. Although the method may be used for any condition in which an NSAID is effective, it is expected that it will be particularly useful in patients with osteoarthritis or rheumatoid arthritis. Other conditions that may be treated include, but are not limited to: all forms of headache, including migraine headache; acute musculoskeletal pain; ankylosing spondylitis; dysmenorrhoea; myalgias; and neuralgias.

In a more general sense, the invention includes methods of treating pain, inflammation and/or other conditions by orally administering an acid inhibitor at a dose effective to raise a patient's gastric pH to at least 3.5, preferably to at least 4 or and more preferably to at least 5. The patient is also administered an NSAID, for example in a coordinated dosage form, that has been coated in a polymer that only dissolves at a pH of at least 3.5, preferably at least 4 and, more preferably, 5 or greater or which dissolves at a rate that is slow enough to prevent NSAID release until after the pH has been raised. When acid inhibitor and NSAID are administered in separate doses, e.g., in two separate tablets, they should be given concomitantly (i.e., so that their biological effects overlap) and may be given concurrently, i.e., NSAID is given within one hour after the acid inhibitor. Preferably, the acid inhibitor is an H2 blocker and, in the most preferred embodiment, it is famotidine at a dosage of between 5 mg and 100 mg. Proton pump inhibitors may also be used and offer advantages in terms of duration of action. Any of the NSAIDs described above may be used in the method but naproxen at a dosage of between 200 and 600 mg is most preferred. It is expected that the acid inhibitor and analgesic will be typically delivered as part of a single unit dosage form which provides for the coordinated release of therapeutic agents. The most preferred dosage form is a multilayer tablet having an outer layer comprising an H2 blocker or a proton pump inhibitor and an inner core comprising an NSAID.

The invention also provides a method for increasing compliance in a patient requiring frequent daily dosing of NSAIDs by providing both an acid inhibitor and NSAID in a single convenient, preferably coordinated, unit dosage form, thereby reducing the number of individual doses to be administered during any given period.

### BRIEF DESCRIPTION OF THE DRAWINGS

FIG. 1 is a schematic diagram of a four layer tablet dosage form. There is a naproxen core layer surrounded by a barrier layer. A third, enteric coating, layer delays the release of naproxen sodium until the pH is at a specific level, e.g., above 4. Finally, there is an outer layer that releases an acid inhibitor such as famotidine.

FIG. 2 illustrates a three layer dosage form. An acid inhibitor, e.g., famotidine, is released immediately after ingestion by a patient in order to raise the pH of the gastrointestinal tract to above a specific pH, e.g., above 4. The innermost layer contains naproxen. Thus, the dosage form has a naproxen core, an enteric film coat and an acid inhibitor film coat.

FIG. 3 illustrates a naproxen sodium pellet which contains a subcoat or barrier coat prior to the enteric film coat.

FIG. 4 illustrates mean pH data over 24 hours on day 9 per protocol population. Treatment A=PN400/E30; B=PN400/E20; C═PN400/E10; D=EC E20+naproxen.

FIG. 5 illustrates mean intragastric pH data over 24 hours on day 1 per protocol population. Treatment A=PN400/E30; B=PN400/E20; C=PN400/E10; D=EC E20+naproxen.

FIG. 6 illustrates mean plasma esomeprazole concentration vs. time curves on day 1.

FIG. 7 illustrates mean plasma esomeprazole concentration vs. time curves on day 9.

FIG. 8 illustrates mean plasma naproxen concentration vs. time curves on day 1.

FIG. 9 illustrates mean naproxen concentration vs. time curves on day 9.

FIG. 10 illustrates the correlation of plasma exposure to esomeprazole with effect on intragastric pH on day 9.

FIG. 11A illustrates mean pH data and mean naproxen concentration vs. time profiles over a 24-hour period on day 9: Treatment B (PN 400/E20).

FIG. 11B illustrates mean pH data and mean naproxen concentration vs. time profiles over a 24-hour period on day 9: Treatment D (EC E20+naproxen).

FIG. 12 illustrates mean plasma naproxen concentration vs. time curves by treatment (All Subjects).

FIG. 13 illustrates mean plasma esomeprazole concentration vs. time curves by treatment.

FIG. 14 illustrates mean plasma concentrations of naproxen and esomeprazole following single dose administration of PN 400/E20 over a 24-hour period. FIG. 14 combines the esomeprazole and naproxen concentration vs. time curves derived for PN 400, as depicted in FIG. 12 (for naproxen) and FIG. 13 (for esomeprazole), at 30 minute timepoints through 4 hours, 2 hour timepoints from 4 to 12 hours, and 4 hour timepoints from 12 to 24 hours. Because FIGS. 12 and 13 present more frequent timepoints, the appearance of the curves in FIG. 14 may differ slightly from their appearance in FIGS. 12 and 13.

### DETAILED DESCRIPTION OF THE INVENTION

Abbreviations and/or special terms that may be used herein are set forth in

Table 1 and the text that follows.

TABLE 1
Abbreviations and Special Terms

| Abbreviation | Explanation |
|---|---|
| ANOVA | analysis of variance |
| AUC | area under the plasma concentration-time curve |
| AUC_{0-10, am} | AUC from time zero (time of AM dosing) to 10 hours after the AM dose |
| AUC_{0-14, pm} | AUC from time zero (time of PM dosing) to 14 hours after the PM dose |
| AUC₀₋₂₄ | AUC from time zero (time of AM dosing) to 24 hours after the AM dose |
| AUC_{0-t, am} | AUC from time zero to the last time point with measurable drug |
| | concentration following AM dosing |
| AUC_{0-t, pm} | AUC from time zero to the last time point with measurable drug |
| | concentration following PM dosing |
| AUC₀₋ₜ | AUC from time zero to the last time point with measurable drug concentration |
| Bid | twice daily |
| BQL | below the lower limit of quantification |
| CBC | complete blood count |
| CI | confidence interval |
| C_{max, am} | maximum plasma concentration after the AM dose |
| C_{max, pm} | maximum plasma concentration after the PM dose |
| CV | coefficient of variation |
| GCP | Good Clinical Practice |
| EC | enteric-coated |
| ECG | electrocardiogram |
| eCRF | electronic case report form |
| Eₘₐₓ | maximal response or pharmacodynamic effect |
| GI | Gastrointestinal |
| GLSM | Geometric least-squares mean |
| HPLC/MS/MS | high pressure liquid chromatography tandem mass spectrometry |
| ITT | intent-to-treat |
| LLOQ | lower limit of quantification |
| LS | least square |
| MedDRA | Medical Dictionary for Regulatory Activities |
| MRM | multiple reaction monitoring |
| λ_{z, am} | apparent first-order elimination rate constant following AM dosing |
| λ_{z, pm} | apparent first-order elimination rate constant following PM dosing |
| Ln | natural log |
| PD | pharmacodynamic(s) |
| PDS | Phoenix Data Systems |
| PK | pharmacokinetic(s) |
| PP | per protocol |
| PPD | Pharmaceutical Product Development |
| PPI | proton pump inhibitor |
| QC | quality control |
| SD | standard deviation |
| SE | standard error |
| SOC | system organ class |
| SPE | solid phase extraction |
| t_{lag, am} | time to the first measurable plasma concentration following the AM dose |
| | (t_{lag, am}) |
| t_{lag, pm} | time to the first measurable plasma concentration following the PM dose |
| | (t_{lag, am}) |
| tₗₐₛₜ | last time point with measurable drug concentration |
| tₘₐₓ | time to maximum plasma concentration |
| t_{1/2, am} | apparent plasma half-life following the AM dose |
| t_{1/2, pm} | apparent plasma half-life following the PM dose |

The term “at risk patient” refers to patient(s) at risk for NSAID associated ulcer due to age or a documented history of gastric ulcers, or receiving concomitant LDA (low dose aspirin). In one embodiment, the at risk patient is a patient at risk for NSAID associated ulcer due to age greater than or equal to 50 years. In another embodiment, the at risk patient is a patient at risk for NSAID associated ulcer due to concomitant aspirin use. In yet another embodiment, the at risk patient is a patient at risk for NSAID associated ulcer due to history of upper gastrointestinal (UGI) ulcer or bleeding.

The term “pharmaceutically-acceptable”, as employed herein, indicates the subject matter being identified as “pharmaceutically acceptable” is suitable and physiologically acceptable for administration to a patient/subject. For example, the term “pharmaceutically acceptable salt(s)” denotes suitable and physiologically acceptable salt(s).

The phrase “naproxen, or pharmaceutically acceptable salt thereof” refers to the free base of naproxen, pharmaceutically acceptable salt(s) of naproxen, and/or mixtures of the free base of naproxen and at least one pharmaceutically acceptable salt of naproxen.

The phrase “esomeprazole, or pharmaceutically acceptable salt thereof” refers to the free base of esomeprazole, pharmaceutically acceptable salt(s) of esomeprazole, and/or mixtures of the free base of esomeprazole and at least one pharmaceutically acceptable salt of esomeprazole.

For the values provided herein, the term “about” indicates a given number may vary by at least 5%, with variations of 10%, 15% or 20% being possible.

With regard to the pharmacokinetic and/or pharmacodynamic values provided herein, the degree of variation is reflected in SDs and % CV values. The % CV=SD/mean×b 100; the SD=(% CV×mean) divided by 100. It can be expected that approximately 68% of patients will be within one SD of the mean and approximately 95% of patients will be within two SDs of the mean. The pharmacokinetic and pharmacodynamic values presented herein are average values, rounded to the nearest whole number, and are based upon results obtained from multiple individuals. As a result, the values presented herein may vary from one patient to another. This variation is reflected in the term “about.”

With regard to the dosages of each of naproxen, or pharmaceutically acceptable salt thereof and/or esomeprazole, or pharmaceutically acceptable salt thereof the term “about” is intended to reflect variations from the specifically identified dosages that are acceptable within the art.

With regard to time periods referred to herein, the term “about” is intended to reflect variations from the specifically identified time periods that are acceptable within the art. With regard to the numerical % coefficient of variation values and/or ranges used herein, the term “about” is intended to reflect variations above and below the stated numerical value and/or range that that may achieve substantially the same results as the stated number. With regard to the pH values and/or ranges recited herein, the term “about” is intended to capture variations above and below the stated number that may achieve substantially the same results as the stated number. With regard to the term numerical values used in conjunction with the phrase “substantially free”, the term is intended to capture variations above and below the stated number that may achieve substantially the same results as the stated number.

The phrase “substantially free” means from about 95% to about 99.99% free. In one embodiment, substantially free means about 95% free. In another embodiment, the term substantially free means about 96% free. In still another embodiment, the term substantially free means about 97% free. In yet another embodiment, the term substantially free means about 98% free. In a further embodiment, the term substantially free means about 99% free. In still a further embodiment, the term substantially free means about 99.99% free.

In the present disclosure, each of the variously stated ranges is intended to be continuous so as to include each numerical parameter between the stated minimum and maximum value of each range. For example, a range of about 1 to about 4 includes about 1, 1, about 2, 2, about 3, 3, about 4, and 4.

The present invention is based upon the discovery of improved pharmaceutical compositions for administering NSAIDs to patients. In addition to containing one or more NSAIDs, the compositions include acid inhibitors that are capable of raising the pH of the GI tract of patients. All of the dosage forms are designed for oral delivery and provide for the coordinated release of therapeutic agents, i.e., for the sequential release of acid inhibitor followed by analgesic.

The NSAIDs used in preparations may be either short or long acting. As used herein, the term “long acting” refers to an NSAID having a pharmacokinetic half-life of at least 2 hours, preferably at least 4 hours and more preferably, at least 8-14 hours. In general, its duration of action will equal or exceed about 6-8 hours. Examples of long-acting NSAIDs are: flurbiprofen with a half-life of about 6 hours; ketoprofen with a half-life of about 2 to 4 hours; naproxen or naproxen sodium with half-lives of about 12 to 15 hours and about 12 to 13 hours respectively; oxaprozin with a half life of about 42 to 50 hours; etodolac with a half-life of about 7 hours; indomethacin with a half-life of about 4 to 6 hours; ketorolac with a half-life of up to about 8-9 hours, nabumetone with a half-life of about 22 to 30 hours; mefenamic acid with a half-life of up to about 4 hours; and piroxicam with a half-life of about 4 to 6 hours. If an NSAID does not naturally have a half-life sufficient to be long acting, it can, if desired, be made long acting by the way in which it is formulated. For example, NSAIDs such as acetaminophen and aspirin may be formulated in a manner to increase their half-life or duration of action. Methods for making appropriate formulations are well known in the art (see e.g. Remington's Pharmaceutical Sciences, 16^{th} ed., A. Oslo editor, Easton, Pa. (1980)).

It is expected that a skilled pharmacologist may adjust the amount of drug in a pharmaceutical composition or administered to a patient based upon standard techniques well known in the art. Nevertheless, the following general guidelines are provided:
Indomethacin is particularly useful when contained in tablets or capsules in an amount from about 25 to 75 mg. A typical daily oral dosage of indomethacin is three 25 mg doses taken at intervals during the day. However, daily dosages of up to about 150 mg are useful in some patients.
Aspirin will typically be present in tablets or capsules in an amount of between about 250 mg and 1000 mg. Typical daily dosages will be in an amount ranging from 500 mg to about 10 g. However, low dose aspirin present at 20-200 mg (and preferably 40-100 mg) per tablet or capsule may also be used.
Ibuprofen may be provided in tablets or capsules of 50, 100, 200, 300, 400, 600, or 800 mg. Daily doses should not exceed 3200 mg. 200 mg-800 mg may be particularly useful when given 3 or 4 times daily.
Flurbiprofen is useful when in tablets at about from 50 to 100 mg. Daily doses of about 100 to 500 mg, and particularly from about 200 to 300 mg, are usually effective.
Ketoprofen is useful when contained in tablets or capsules in an amount of about 25 to 75 mg. Daily doses of from 100 to 500 mg and particularly of about 100 to 300 mg are typical as is about 25 to 50 mg every six to eight hours.
Naproxen is particularly useful when contained in tablets or capsules in an amount of from 250 to 500 mg. For naproxen sodium, tablets of about 275 or about 550 mg are typically used. Initial doses of from 100 to 1250 mg, and particularly 350 to 800 mg are also used, with doses of about 550 mg being generally preferred.
Oxaprozin may be used in tablets or capsules in the range of roughly 200 mg to 1200 mg, with about 600 mg being preferred. Daily doses of 1200 mg have been found to be particularly useful and daily doses should not exceed 1800 mg or 26 mg/kg.
Etodolac is useful when provided in capsules of 200 mg to 300 mg or in tablets of about 400 mg. Useful doses for acute pain are 200-400 mg every six-eight hours, not to exceed 1200 mg/day. Patients weighing less than 60 kg are advised not to exceed doses of 20 mg/kg. Doses for other uses are also limited to 1200 mg/day in divided doses, particularly 2, 3 or 4 times daily.
Ketorolac is usefully provided in tablets of 1-50 mg, with about 10 mg being typical. Oral doses of up to 40 mg, and particularly 10-30 mg/day have been useful in the alleviation of pain.
Nabumetone may be provided in tablets or capsules of between 500 mg and 750 mg. Daily doses of 1500-2000 mg, after an initial dose of 100 mg, are of particular use.
Mefenamic acid is particularly useful when contained in tablets or capsules of 50 mg to 500 mg, with 250 mg being typical. For acute pain, an initial dosage of 1-1000 mg, and particularly about 500 mg, is useful, although other doses may be required for certain patients.
Lornoxicam is provided in tablets of 4 mg or 8 mg. Useful doses for acute pain are 8 mg or 16 mg daily, and for arthritis are 12 mg daily.

Other NSAIDs that may be used include: celecoxib, rofecoxib, meloxicam, piroxicam, droxicam, tenoxicam, valdecoxib, parecoxib, etoricoxib, CS-502, JTE-522, L-745,337, or NS398. JTE-522, L-745,337 and NS398 as described, inter alia, in Wakatani, et al. (Jpn. J. Pharmacol. 78:365-371 (1998)) and Panara, et al. (Br. J. Pharmacol. 116:2429-2434 (1995)). The amount present in a tablet or administered to a patient will depend upon the particular NSAID being used. For example:
Celecoxib may be administered in a tablet or capsule containing from about 100 mg to about 500 mg or, preferably, from about 100 mg to about 200 mg.
Piroxicam may be used in tablets or capsules containing from about 10 to 20 mg.
Rofecoxib will typically be provided in tablets or capsules in an amount of 12.5, or 50 mg. The recommended initial daily dosage for the management of acute pain is 50 mg.
Meloxicam is provided in tablets of 7.5 mg, with a recommended daily dose of 7.5 or 15 mg for the management of osteoarthritis.
Valdecoxib is provided in tablets of 10 or 20 mg, with a recommended daily dose of 10 mg for arthritis or 40 mg for dysmenorrhea.

With respect to acid inhibitors, tablets or capsules may contain anywhere from 1 mg to as much as 1 g. Typical amounts for H2 blockers are: cimetidine, 100 to 800 mg/unit dose; ranitidine, 50-300 mg/unit dose; famotidine, 5-100 mg/unit dose; ebrotidine 400-800 mg/unit dose; pabutidine 40 mg/unit dose; lafutidine 5-20 mg/unit dose; and nizatidine, 50-600 mg/unit dose. Proton pump inhibitors will typically be present at about 5 mg to 600 mg per unit dose. For example, the proton pump inhibitor omeprazole should be present in tablets or capsules in an amount from 5 to 50 mg, with about 10 or 20 mg being preferred. Other typical amounts are: esomeprazole, 5-100 mg, with about 40 mg being preferred; lansoprazole, 5-150 mg (preferably 5-50 mg), with about 7.5, 15 or 30 mg being most preferred; pantoprazole, 10-200 mg, with about 40 mg being preferred; and rabeprazole, 5-100 mg, with about 20 mg being preferred.

### Making of Pharmaceutical Preparations

The pharmaceutical compositions of the invention include tablets, dragees, liquids and capsules and can be made in accordance with methods that are standard in the art (see, e.g., Remington's Pharmaceutical Sciences, 16^{th} ed., A Oslo editor, Easton, Pa. (1980)). Drugs and drug combinations will typically be prepared in admixture with conventional excipients. Suitable carriers include, but are not limited to: water; salt solutions; alcohols; gum arabic; vegetable oils; benzyl alcohols; polyethylene glycols; gelatin; carbohydrates such as lactose, amylose or starch; magnesium stearate; talc; silicic acid; paraffin; perfume oil; fatty acid esters; hydroxymethylcellulose; polyvinyl pyrrolidone; etc. The pharmaceutical preparations can be sterilized and, if desired, mixed with auxiliary agents such as: lubricants, preservatives, disintegrants; stabilizers; wetting agents; emulsifiers; salts; buffers; coloring agents; flavoring agents; or aromatic substances.

Enteric coating layer(s) may be applied onto the core or onto the barrier layer of the core using standard coating techniques. The enteric coating materials may be dissolved or dispersed in organic or aqueous solvents and may include one or more of the following materials: methacrylic acid copolymers, shellac, hydroxypropylmethcellulose phthalate, polyvinyl acetate phthalate, hydroxypropylmethylcellulose trimellitate, carboxymethylethyl-cellulose, cellulose acetate phthalate or other suitable enteric coating polymer(s). The pH at which the enteric coat will dissolve can be controlled by the polymer or combination of polymers selected and/or ratio of pendant groups. For example, dissolution characteristics of the polymer film can be altered by the ratio of free carboxyl groups to ester groups. Enteric coating layers also contain pharmaceutically acceptable plasticizers such as triethyl citrate, dibutyl phthalate, triacetin, polyethylene glycols, polysorbates or other plasticizers. Additives such as dispersants, colorants, anti-adhering and anti-foaming agents may also be included.

### The Making of Tablet Dosage Forms

Preferably, the combination of an acid inhibitor and an NSAID will be in the form of a bi- or multi-layer tablet. In a bilayer configuration, one portion of the tablet contains the acid inhibitor in the required dose along with appropriate excipients, agents to aid dissolution, lubricants, fillers, etc. The second portion of the tablet will contain the NSAID, preferably naproxen, in the required dose along with other excipients, dissolution agents, lubricants, fillers, etc. In the most preferred embodiment, the NSAID layer is surrounded by a polymeric coating which does not dissolve at a pH of less than 4. The NSAID may be granulated by methods such as slugging, low- or high-shear granulation, wet granulation, or fluidized-bed granulation. Of these processes, slugging generally produces tablets of less hardness and greater friability. Low-shear granulation, high-shear granulation, wet granulation and fluidized-bed granulation generally produce harder, less friable tablets.

### Pharmacokinetics of Disclosed Compositions

In one embodiment, the disclosure is directed to the use of naproxen, or pharmaceutically acceptable salt thereof, and esomeprazole, or pharmaceutically acceptable salt thereof in the manufacture of a pharmaceutical composition in unit dose form wherein said esomeprazole, or pharmaceutically acceptable salt thereof, is released from said unit dose form at a pH of from about 0 or greater to target: a mean % time at which intragastric pH remains at about 4.0 or greater for about a 24 hour period of at least about 41%.

In another embodiment, the mean % time at which intragastric pH remains at about 4.0 or greater for about a 24 hour period is at least about 60%, at least about 71%, or at least about 77%.

In another embodiment, the pharmaceutical composition further targets a pharmacokinetic (pk) profile having a mean maximum plasma concentration (Cₘₐₓ) for naproxen of about 76 μg/ml or about 79 μg/ml.

In another embodiment, the pharmaceutical composition targets a pharmacokinetic (pk) profile having a mean area under the plasma concentration-time curve from time zero when first dose is administered to about 24 hours after the first dose is administered (AUC₀₋₂₄) for esomeprazole of about 1500 hr*μg/mL, of about 2134 hr*μg/mL or about 4911 hr*μg/mL.

In one aspect, the disclosure is directed to the use of a naproxen, or pharmaceutically acceptable salt thereof, and esomeprazole, or pharmaceutically acceptable salt thereof in the manufacture of a pharmaceutical composition in unit dose form for delivery to a patient in need thereof, wherein a first unit dose form is administered as an AM dose and a second unit dose form is administered about 10 hours later as a PM dose to target:
i) a pharmacokinetic (pk) profile for naproxen where:
   a) the AM dose has a mean Cₘₐₓ of about 81 μg/mL and a median time to maximum concentration (Tₘₐₓ) of from about 2.5 to about 4 hours, and
   b) the PM dose has a mean Cₘₐₓ of about 76.2 μg/mL and a median Tₘₐₓ of from about 10 to about 14 hours; and
ii) a pharmacokinetic (pk) profile for esomeprazole where:
   a) the AM dose has a mean area under the plasma concentration-time curve from time zero when the AM dose is administered to about 10 hours after the AM dose is administered (AUC_{0-10,am}) of about 850 hr*μg/mL, and
   b) the PM dose has a mean area under the plasma concentration-time curve from time zero when the PM dose is administered to about 14 hours after the PM dose is administered (AUC_{0-14,pm}) of about 650 hr*μm/mL.

In one embodiment, the pharmaceutical composition provides for:
i) the mean Cₘₐₓ for said AM dose of naproxen is about 86.2 μg/mL or about 80.9 μg/mL and said median Tₘₐₓ is about 3.0 hours; and
ii) the mean Cₘₐₓ for said PM dose is about 76.8 μg/mL or about 76.2 μg/mL and said median Tₘₐₓ is about 10 hours.

In another embodiment, the pharmaceutical composition described above provides for:
i) the mean area under the plasma concentration-time curve from time zero when the AM dose is administered to about 10 hours after the AM dose is administered (AUC_{0-10,am}) for said AM dose of esomeprazole is about 1216 hr*μg/mL or about 2779 hr*μg/mL, and
ii) the mean area under the plasma concentration-time curve from time zero when the PM dose is administered to about 14 hours after the PM dose is administered (AUC_{0-14,pm}) for said PM dose of esomeprazole is about 919 hr*μg/mL or about 2066 hr*μg/mL.

In another embodiment, the pharmaceutical composition targets a pharmacokinetic (pk) profile having a mean area under the plasma concentration-time curve from time zero when first dose is administered to about 24 hours after the first dose is administered (AUC₀₋₂₄) for esomeprazole is about 1500 hr*μg/mL, about 2000 hr*μg/mL, or about 4911 hr*μg/mL.

In other embodiments, the pharmaceutical composition further targets a mean % time at which intragastric pH remains at about 4.0 or greater for about a 24 hour period after reaching steady state of at least about 41%, about 60%, about 71%, or about 77%.

Steady state levels of naproxen or esomeprazole are generally reached in about 4 to 5 days, about 5 to 6 days, about 6 to 7 days, about 7 to 8 days, about 8 to 9 days, or about 9 to 10 days after twice daily delivery of the pharmaceutical compositions in unit dose form disclosed herein. In other embodiments, steady state levels of naproxen or esomeprazole are reached in about 4 days, about 5 days, about 6 days, about 7 days, about 8 days, about 9 days, or about 10 days.

In one embodiment, the dose form is a multilayer tablet comprising at least one core and at least a first layer and a second layer, wherein:
i) said core comprises naproxen, or pharmaceutically acceptable salt thereof;
ii) said first layer is a coating that at least begins to release the naproxen, or pharmaceutically acceptable salt thereof, when the pH of the surrounding medium is about 3.5 or greater; and
iii) said second layer comprises esomeprazole, wherein said esomeprazole is released at a pH of from about 0 or greater.

In another embodiment, the does form provides for the release of esomeprazole at a pH of from about 1 or greater, a pH of from about 0 to about 2, or at a pH of about 2 to 4. In another embodiment, at least a portion of the esomeprazole, or pharmaceutically acceptable salt thereof, is not coated with an enteric coating. In another embodiment, the first layer is an enteric coating. In yet another embodiment, the multi-layer tablet is substantially free of sodium bicarbonate. In one embodiment, the first layer begins to release the naproxen when the pH of the surrounding medium is at about 4.0 or about 4.5 or greater.

In another embodiment, the pharmaceutical composition in unit dose form comprises about 500 mg of said naproxen, or pharmaceutically acceptable salt thereof, and about 20 mg or about 30 mg, of said esomeprazole, or pharmaceutically acceptable salt thereof.

In another embodiment, the pharmaceutical composition is administered twice a day for at least about 6 days or twice a day for at least 9 days.

In another embodiment, the pharmaceutical composition is for administration to an at risk patient. The at risk patient can be a patient being treated for a disease or disorder selected from pain and inflammation, or can be a patient being treated for osteoarthritis, rheumatoid arthritis, ankylosing spondylitis, or a combination thereof.

One embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater to target: a mean % time at which intragastric pH remains at about 4.0 or greater for about a 24 hour period after reaching steady state of at least about 41%.

In other embodiments, the mean % time at which intragastric pH remains at about 4.0 or greater for a 24 hour period after reaching steady state is at least about 40%, about 45%, about 50%, about 55%, about 60%, about 65%, about 70%, about 71%, about 75%, about 77%, about 80%, about 85%, about 90%, or about 95%.

Another embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater to target: a mean % time at which intragastric pH remains at about 4.0 or greater for about a 24 hour period after reaching steady state of at least about 41% and a pharmacokinetic (pk) profile having a mean maximum plasma concentration (Cₘₐₓ) for naproxen of at least 76 μg/mL with a coefficient of variation ranging from 17-23.

A further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater to target: a mean % time at which intragastric pH remains at about 4.0 or greater for about a 24 hour period after reaching steady state of at least about 41% and a pharmacokinetic (pk) profile having a mean maximum plasma concentration (Cₘₐₓ) for naproxen of about 76 μg/mL.

Yet another embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from 0 or greater to target: a mean % time at which intragastric pH remains at about 4.0 or greater for about a 24 hour period after reaching steady state of at least about 41% and a pharmacokinetic (pk) profile having a mean area under the plasma concentration-time curve from time zero when first dose is administered to about 24 hours after the first dose is administered (AUC₀₋₂₄) for esomeprazole of at least 1500 hr*μg/mL with a % coefficient of variation ranging from 40-80.

Yet a further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from 0 or greater to target: a mean % time at which intragastric pH remains at about 4.0 or greater for about a 24 hour period after reaching steady state of at least about 41% and a pharmacokinetic (pk) profile having a mean area under the plasma concentration-time curve from time zero when first dose is administered to about 24 hours after the first dose is administered (AUC₀₋₂₄) for esomeprazole of about 1500 hr*μg/mL.

Still yet another embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater to target: a mean time at which intragastric pH remains at about 4.0 or greater for about a 24 hour period after reaching steady state of at least about 41%; and a pharmacokinetic (pk) profile having a mean maximum plasma concentration (Cₘₐₓ) for naproxen of at least 76 μg/mL with a coefficient of variation ranging from 17-23 and a mean area under the plasma concentration-time curve from time zero when first dose is administered to about 24 hours after the first dose is administered (AUC₀₋₂₄) for esomeprazole of at least 1500 hr*μg/mL with a % coefficient of variation ranging from 40-80.

Still even yet another embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater to target: a mean % time at which intragastric pH remains at about 4.0 or greater for about a 24 hour period after reaching steady state of at least about 41%; and a pharmacokinetic (pk) profile having a mean maximum plasma concentration (Cₘₐₓ) for naproxen of about 76 μg/mL and a mean area under the plasma concentration-time curve from time zero when first dose is administered to about 24 hours after the first dose is administered (AUC₀₋₂₄) for esomeprazole of about 1500 hr*μg/mL.

A still even further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater to target: a pharmacokinetic (pk) profile having a mean maximum plasma concentration (Cₘₐₓ) for naproxen of at least 76 μg/mL with a % coefficient of variation ranging from 17-23.

Yet a further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater to target: a pharmacokinetic (pk) profile having a mean maximum plasma concentration (Cₘₐₓ) for naproxen of about 76 μg/mL.

A yet still further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater to target: and a pharmacokinetic (pk) profile having mean area under the plasma concentration-time curve from time zero when first dose is administered to about 24 hours after the first dose is administered (AUC₀₋₂₄) for esomeprazole of at least 1500 hr*μg/mL with a % coefficient of variation ranging from 40-80.

A yet even further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater to target: and a pharmacokinetic (pk) profile having mean area under the plasma concentration-time curve from time zero when first dose is administered to about 24 hours after the first dose is administered (AUC₀₋₂₄) for esomeprazole of about 1500 hr*μg/mL.

A further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of at least 81 μg/mL with a % coefficient of variation ranging from 22-23 and a median time to maximum concentration (Tₘₐₓ) of from about 2.5 to about 4 hours, and
   b) the PM dose has a mean Cₘₐₓ of at least 76.2 μg/mL with a % coefficient of variation ranging from 18-23 and a median Tₘₐₓ of from about 10 to about 14 hours; and
ii) a pk profile for esomeprazole where:
   a) the AM dose has a mean area under the plasma concentration-time curve from time zero when the AM dose is administered to about 10 hours after the AM dose is administered (AUC_{0-10,am}) of at least 850 hr*μg/mL with a % coefficient of variation ranging from 45-70, and
   b) the PM dose has a mean area under the plasma concentration-time curve from time zero when the PM dose is administered to about 14 hours after the PM dose is administered (AUC_{0-14,pm}) of at least 650 hr*μg/mL with a % coefficient of variation ranging from 50-85.

A yet further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from 0 or greater, wherein one unit dose form is administered as an AM dose and a second dose administered about 10 hours later as a PM dose to target:
i) a pk profile for naproxen where:
   a) the AM dose has a mean Cₘₐₓ of about 81 μg/mL and a median time to maximum concentration (Tₘₐₓ) of from about 2.5 to about 4 hours, and
   b) the PM dose has a mean Cₘₐₓ of about 76.2 μg/mL and a median Tₘₐₓ of from about 10 to about 14 hours; and
ii) a pk profile for esomeprazole where:
   a) the AM dose has a mean area under the plasma concentration-time curve from time zero when the AM dose is administered to about 10 hours after the AM dose is administered (AUC_{0-10,am}) of about 850 hr*μg/mL, and
   b) the PM dose has a mean area under the plasma concentration-time curve from time zero when the PM dose is administered to about 14 hours after the PM dose is administered (AUC_{0-14,pm}) of about 650 hr*μg/mL.

A still further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of at least 81 μg/mL with a % coefficient of variation ranging from 22-23 and a median time to maximum concentration (Tₘₐₓ) of from about 2.5 to about 4 hours, and
   b) the PM dose has a mean Cₘₐₓ of at least 76.2 μg/mL with a % coefficient of variation ranging from 18-23 and a median Tₘₐₓ of from about 10 to about 14 hours.

A yet still further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of about 81 μg/mL and a median time to maximum concentration (Tₘₐₓ) of from about 2.5 to about 4 hours, and
   b) the PM dose has a mean Cₘₐₓ of about 76.2 μg/mL and a median Tₘₐₓ of from about 10 to about 14 hours.

A yet even still further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for esomeprazole where:
   a) the AM dose has a mean area under the plasma concentration-time curve from time zero when the AM dose is administered to about 10 hours after the AM dose is administered (AUC_{0-10,am}) is at least 850 hr*μg/mL with a % coefficient of variation ranging from 45-70, and b) the PM dose has a mean area under the plasma concentration-time curve from time zero when the PM dose is administered to about 14 hours after the PM dose is administered (AUC_{0-14,pm}) is at least 650 hr*μg/mL with a % coefficient of variation ranging from 50-85.

A further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for esomeprazole where:
   a) the AM dose has a mean area under the plasma concentration-time curve from time zero when the AM dose is administered to about 10 hours after the AM dose is administered (AUC_{0-10,am}) is about 850 hr*μg/mL, and
   b) the PM dose has a mean area under the plasma concentration-time curve from time zero when the PM dose is administered to about 14 hours after the PM dose is administered (AUC_{0-14,pm}) of about 650 hr*μg/mL.

A yet still even further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of at least 81 μg/mL with a % coefficient of variation ranging from 22-23 and a median time to maximum concentration (Tₘₐₓ) of from about 2.5 to about 4 hours, and
   b) the PM dose has a mean Cₘₐₓ of at least 76.2 μg/mL with % coefficient of variation ranging from 18-23 and a median Tₘₐₓ of from about 10 to about 14 hours;
ii) a pk profile for esomeprazole where:
   a) the AM dose has a mean area under the plasma concentration-time curve from time zero when the AM dose is administered to about 10 hours after the AM dose is administered (AUC_{0-10,am}) is at least 850 hr*μg/mL with a % coefficient of variation ranging from 45-70, and b) the PM dose has a mean area under the plasma concentration-time curve from time zero when the PM dose is administered to about 14 hours after the PM dose is administered (AUC_{0-14,pm}) is at least 650 hr*μg/mL with a % coefficient of variation ranging from 50-85; and
iii) a mean % time at which intragastric pH remains at about 4.0 or greater for about a 24 hour period after reaching steady state of at least about 41%.

A still even further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of about least 81 μg/mL and a median time to maximum concentration (Tₘₐₓ) of from about 2.5 to about 4 hours, and
   b) the PM dose has a mean Cₘₐₓ of about 76.2 μg/mL and a median Tₘₐₓ of from about 10 to about 14 hours;
ii) a pk profile for esomeprazole where:
   a) the AM dose has a mean area under the plasma concentration-time curve from time zero when the AM dose is administered to about 10 hours after the AM dose is administered (AUC_{0-10,am}) of about 850 hr*μg/mL, and
   b) the PM dose has a mean area under the plasma concentration-time curve from time zero when the PM dose is administered to about 14 hours after the PM dose is administered (AUC_{0-14,pm}) of about 650 hr*μg/mL; and
iii) a mean % time at which intragastric pH remains at about 4.0 or greater for about a 24 hour period after reaching steady state of at least about 41%.

Still yet another embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of at least 81 μg/mL with a coefficient of variation ranging from 22-23 and a median time to maximum concentration (Tₘₐₓ) of from about 2.5 to about 4 hours, and
   b) the PM dose has a mean Cₘₐₓ of at least 76.2 μg/mL with a % coefficient of variation ranging from 18-23 and a median Tₘₐₓ of from about 10 to about 14 hours; and
ii) a mean % time at which intragastric pH remains at about 4.0 or greater for about a 24 hour period after reaching steady state of at least about 41%.

A still yet further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of about 81 μg/mL and a median time to maximum concentration (Tₘₐₓ) of from about 2.5 to about 4 hours, and
   b) the PM dose has a mean Cₘₐₓ of about 76.2 μg/mL and a median Tₘₐₓ of from about 10 to about 14 hours; and
ii) a mean % time at which intragastric pH remains at about 4.0 or greater for about a 24 hour period after reaching steady state of at least about 41%.

Even still yet a further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole is released from said unit dose form at a pH of from 0 or greater, wherein one unit dose form is administered as an AM dose and a second dose administered about 10 hours later as a PM dose to target:
i) a pk profile for esomeprazole where:
   a) the AM dose has a mean area under the plasma concentration-time curve from time zero when the AM dose is administered to about 10 hours after the AM dose is administered (AUC_{0-10,am}) is at least 850 hr*μg/mL with a % coefficient of variation ranging from 45-70, and b) the PM dose has a mean area under the plasma concentration-time curve from time zero when the PM dose is administered to about 14 hours after the PM dose is administered (AUC_{0-14,pm}) is at least 650 hr*μg/mL with a % coefficient of variation ranging from 50-85; and
ii) a mean % time at which intragastric pH remains at about 4.0 or greater for about a 24 hour after reaching steady state period of at least about 41%.

An even still further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole is released from said unit dose form at a pH of from 0 or greater, wherein one unit dose form is administered as an AM dose and a second dose administered about 10 hours later as a PM dose to target:
i) a pk profile for esomeprazole where:
   a) the AM dose has a mean area under the plasma concentration-time curve from time zero when the AM dose is administered to about 10 hours after the AM dose is administered (AUC_{0-10,am}) of about 850 hr*μg/mL, and
   b) the PM dose has a mean area under the plasma concentration-time curve from time zero when the PM dose is administered to about 14 hours after the PM dose is administered (AUC_{0-14,pm}) of about 650 hr*μg/mL; and
ii) a mean % time at which intragastric pH remains at about 4.0 or greater for about a 24 hour period after reaching steady state of at least about 41%.

A yet still even further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of at least 81 μg/mL with a % coefficient of variation ranging from 22-23 and a median time to maximum concentration (Tₘₐₓ) of from about 2.5 to about 4 hours, and
   b) the PM dose has a mean Cₘₐₓ of at least 76.2 μg/mL with a % coefficient of variation of ranging from 18-23 and a median Tₘₐₓ of from about 10 to about 14 hours;
ii) a pk profile for esomeprazole where:
   a) the AM dose has a mean area under the plasma concentration-time curve from time zero when the AM dose is administered to 10 hours after the AM dose is administered (AUC_{0-10,am}) is at least 850 hr*μg/mL with a % coefficient of variation ranging from 45-70, and b) the PM dose has a mean area under the plasma concentration-time curve from time zero when the PM dose is administered to about 14 hours after the PM dose is administered (AUC_{0-14,pm}) is at least 650 hr*μg/mL with a % coefficient of variation ranging from 50-85;
iii) a mean % time at which intragastric pH remains at about 4.0 or greater for about a 24 hour period after reaching steady state of at least about 41%; and
iv) a pharmacokinetic (pk) profile having a mean area under the plasma concentration-time curve from time zero when first dose is administered to about 24 hours after the first dose is administered (AUC₀₋₂₄) for esomeprazole of at least 1500 hr*μg/mL with a % coefficient of variation ranging from 40-80.

Another embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of about least 81 μg/mL and a median time to maximum concentration (Tₘₐₓ) of from about 2.5 to about 4 hours, and
   b) the PM dose has a mean Cₘₐₓ of about 76.2 μg/mL and a median Tₘₐₓ of from about 10 to about 14 hours;
ii) a pk profile for esomeprazole where:
   a) the AM dose has a mean area under the plasma concentration-time curve from time zero when the AM dose is administered to about 10 hours after the AM dose is administered (AUC_{0-10,am}) of about 850 hr*μg/mL, and
   b) the PM dose has a mean area under the plasma concentration-time curve from time zero when the PM dose is administered to about 14 hours after the PM dose is administered (AUC_{0-14,pm}) of about 650 hr*μg/mL;
iii) a mean % time at which intragastric pH remains at about 4.0 or greater for a 24 hour period after reaching steady state of at least about 41%; and
iv) a pharmacokinetic (pk) profile having a mean area under the plasma concentration-time curve from time zero when first dose is administered to about 24 hours after the first dose is administered (AUC₀₋₂₄) for esomeprazole of about 1500 hr*μg/mL.

A further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of at least 81 μg/mL with a % coefficient of variation ranging from 22-23 and a median time to maximum concentration (Tₘₐₓ) of from about 2.5 to about 4 hours, and
   b) the PM dose has a mean Cₘₐₓ of at least 76.2 μg/mL with a % coefficient of variation ranging from 18-23 and a median Tₘₐₓ of from about 10 to about 14 hours;
ii) a pk profile for esomeprazole where:
   a) the AM dose has a mean area under the plasma concentration-time curve from time zero when the AM dose is administered to about 10 hours after the AM dose is administered (AUC_{0-10,am}) is at least 850 hr*μg/mL with a % coefficient of variation ranging from 45-70, and
   b) the PM dose has a mean area under the plasma concentration-time curve from time zero when the PM dose is administered to about 14 hours after the PM dose is administered (AUC_{0-14,pm}) is at least 650 hr*μg/mL with a % coefficient of variation ranging from 50-85; and
iii) a pharmacokinetic (pk) profile having a mean area under the plasma concentration-time curve from time zero when first dose is administered to about 24 hours after the first dose is administered (AUC₀₋₂₄) for esomeprazole of at least 1500 hr*μg/mL with a % coefficient of variation ranging from 40-80.

A yet further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of about 81 μg/mL and a median time to maximum concentration (Tₘₐₓ) of from about 2.5 to about 4 hours, and
   b) the PM dose has a mean Cₘₐₓ of about 76.2 μg/mL and a median Tₘₐₓ of from about 10 to about 14 hours;
ii) a pk profile for esomeprazole where:
   a) the AM dose has a mean area under the plasma concentration-time curve from time zero when the AM dose is administered to about 10 hours after the AM dose is administered (AUC_{0-10,am}) of about 850 hr*μg/mL, and
   b) the PM dose has a mean area under the plasma concentration-time curve from time zero when the PM dose is administered to about 14 hours after the PM dose is administered (AUC_{0-14,pm}) of about 650 hr*μg/mL; and
iii) a pharmacokinetic (pk) profile having a mean area under the plasma concentration-time curve from time zero when first dose is administered to about 24 hours after the first dose is administered (AUC₀₋₂₄) for esomeprazole of about 1500 hr*μg/mL.

A still further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for esomeprazole where:
   a) the AM dose has a mean area under the plasma concentration-time curve from time zero when the AM dose is administered to about 10 hours after the AM dose is administered (AUC_{0-10,am}) is at least 850 hr*μg/mL with a % coefficient of variation ranging from 45-70, and
   b) the PM dose has a mean area under the plasma concentration-time curve from time zero when the PM dose is administered to about 14 hours after the PM dose is administered (AUC_{0-14,pm}) is at least 650 hr*μg/mL with a % coefficient of variation ranging from 50-85; and
ii) a pharmacokinetic (pk) profile having a mean area under the plasma concentration-time curve from time zero when first dose is administered to about 24 hours after the first dose is administered (AUC₀₋₂₄) for esomeprazole is at least 1500 hr*μg/mL with a % coefficient of variation ranging from 40-80.

Still a further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for esomeprazole where:
   a) the AM dose has a mean area under the plasma concentration-time curve from time zero when the AM dose is administered to about 10 hours after the AM dose is administered (AUC_{0-10,am}) of about 850 hr*μg/mL, and
   b) the PM dose has a mean area under the plasma concentration-time curve from time zero when the PM dose is administered to about 14 hours after the PM dose is administered (AUC_{0-14,pm}) of about 650 hr*μg/mL; and
ii) a pharmacokinetic (pk) profile having a mean area under the plasma concentration-time curve from time zero when first dose is administered to about 24 hours after the first dose is administered (AUC₀₋₂₄) for esomeprazole of about 1500 hr*μg/mL.

Yet another embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of at least 81 μg/mL with a % coefficient of variation ranging from 22-23 and a median time to maximum concentration (Tₘₐₓ) of from about 2.5 to about 4 hours, and
   b) the PM dose has a mean Cₘₐₓ of at least 76.2 μg/mL with a % coefficient of variation ranging from 18-23 and a median Tₘₐₓ of from about 10 to about 14 hours; and
ii) a pharmacokinetic (pk) profile having a mean area under the plasma concentration-time curve from time zero when first dose is administered to about 24 hours after the first dose is administered (AUC₀₋₂₄) for esomeprazole is at least 1500 hr*μg/mL with a % coefficient of variation ranging from 40-80.

Yet still another embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of about 81 μg/mL and a median time to maximum concentration (Tₘₐₓ) of from about 2.5 to about 4 hours, and
   b) the PM dose has a mean Cₘₐₓ of about 76.2 μg/mL and a median Tₘₐₓ of from about 10 to about 14 hours; and
ii) a pharmacokinetic (pk) profile having a mean area under the plasma concentration-time curve from time zero when first dose is administered to about 24 hours after the first dose is administered (AUC₀₋₂₄) for esomeprazole of about 1500 hr*μg/mL.

Yet a further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for esomeprazole where:
   a) the AM dose has a mean area under the plasma concentration-time curve from time zero when the AM dose is administered to about 10 hours after the AM dose is administered (AUC_{0-10,pm}) is at least 850 hr*μg/mL with a % coefficient of variation ranging from 45-70, and b) the PM dose has a mean area under the plasma concentration-time curve from time zero when the PM dose is administered to about 14 hours after the PM dose is administered (AUC_{0-14,pm}) is at least 650 hr*μg/mL with a % coefficient of variation ranging from 50-85; ii) a mean % time at which intragastric pH remains at about 4.0 or greater for about a 24 hour period after reaching steady state of at least about 41%; and
iii) a pharmacokinetic (pk) profile having a mean area under the plasma concentration-time curve from time zero when first dose is administered to about 24 hours after the first dose is administered (AUC₀₋₂₄) for esomeprazole is at least 1500 hr*μg/mL with a % coefficient of variation ranging from 40-80.

Yet a still further embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for esomeprazole where:
   a) the AM dose has a mean area under the plasma concentration-time curve from time zero when the AM dose is administered to about 10 hours after the AM dose is administered (AUC_{0-10,am}) of about 850 hr*μg/mL, and
   b) the PM dose has a mean area under the plasma concentration-time curve from time zero when the PM dose is administered to about 14 hours after the PM dose is administered (AUC_{0-14,pm}) of about 650 hr*μg/mL;
ii) a mean % time at which intragastric pH remains at about 4.0 or greater for about a 24 hour period after reaching steady state of at least about 41%; and
iii) a pharmacokinetic (pk) profile having a mean area under the plasma concentration-time curve from time zero when first dose is administered to about 24 hours after the first dose is administered (AUC₀₋₂₄) for esomeprazole of about 1500 hr*μg/mL.

Yet another embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of at least 81 μg/mL with a coefficient of variation ranging from 22-23 and a median time to maximum concentration (Tₘₐₓ) of from about 2.5 to about 4 hours, and
   b) the PM dose has a mean Cₘₐₓ of at least 76.2 μg/mL with a coefficient of variation ranging from 18-23 and a median Tₘₐₓ of from about 10 to about 14 hours;
ii) a mean % time at which intragastric pH remains at about 4.0 or greater for about a 24 hour period after reaching steady state of at least about 41%; and
iii) a pharmacokinetic (pk) profile having a mean area under the plasma concentration-time curve from time zero when first dose is administered to about 24 hours after the first dose is administered (AUC₀₋₂₄) for esomeprazole is at least 1500 hr*μg/mL with a % coefficient of variation ranging from 40-80.

Yet still another embodiment is directed to a method for delivering a pharmaceutical composition to a patient in need thereof, comprising: administering to said patient a pharmaceutical composition in unit dose form comprising naproxen, or pharmaceutically acceptable salt thereof, and esomeprazole, or pharmaceutically acceptable salt thereof, wherein said esomeprazole, or pharmaceutically acceptable salt thereof, is released from said unit dose form at a pH of from about 0 or greater, wherein one unit dose form is administered as an AM dose and a second dose administered about 10 hours later as a PM dose to target:
i) a pk profile for naproxen where:
   a) the AM dose has a mean Cₘₐₓ of about 81 μg/mL and a median time to maximum concentration (Tₘₐₓ) of from about 2.5 to about 4 hours, and
   b) the PM dose has a mean Cₘₐₓ of about 76.2 μg/mL and a median Tₘₐₓ of from about 10 to about 14 hours;
ii) a mean % time at which intragastric pH remains at about 4.0 or greater for about a 24 hour period after reaching steady state of at least about 41%; and
iii) a pharmacokinetic (pk) profile having a mean area under the plasma concentration-time curve from time zero when first dose is administered to about 24 hours after the first dose is administered (AUC₀₋₂₄) for esomeprazole is about 1500 hr*μg/mL.

In another embodiment, naproxen can be present as the free base in an amount of from about 500 mg. In still another embodiment, naproxen can be present as the free base in an amount of about 500 mg. In yet another embodiment, naproxen can be present in equivalent amounts of pharmaceutically acceptable salts of naproxen, e.g., sodium naproxen. In a further embodiment, esomeprazole can be present as a magnesium salt.

In an even further embodiment, esomeprazole, or pharmaceutically acceptable salt thereof, can be present in an amount to provide from about 10 mg to about 30 mg of esomeprazole. In a further embodiment, esomeprazole, or pharmaceutically acceptable salt thereof, can be present in an amount to provide about 15 mg of esomeprazole. In yet an even further embodiment, esomeprazole, or pharmaceutically acceptable salt thereof, can be present in an amount to provide about 20 mg of esomeprazole. In still yet another embodiment, esomeprazole, or pharmaceutically acceptable salt thereof, can be present in an amount to provide about 30 mg of esomeprazole.

In still another embodiment, the mean % time at which intragastric pH remains at about 4.0 or greater for about a 24 hour period after reaching steady state is at least about 60%. In yet another embodiment, the mean % time at which intragastric pH remains at about 4.0 or greater for about a 24 hour period after reaching steady state is at least about 71%. In still yet an even further embodiment, the mean % time at which intragastric pH remains at about 4.0 or greater for about a 24 hour period after reaching steady state is at least about 77%.

In an even still further embodiment, the mean maximum plasma concentration (Cₘₐₓ) for naproxen is at least 79 μg/mL with a % coefficient of variation ranging from 17-23. In a further embodiment, the mean maximum plasma concentration (Cₘₐₓ) for naproxen is about 79 μg/mL.

In yet an even still further embodiment, the mean area under the plasma concentration-time curve from time zero when first dose is administered to about 24 hours after the first dose is administered (AUC₀₋₂₄) for esomeprazole is 2134 hr*μg/mL with a coefficient of variation of 74. In another embodiment, the mean area under the plasma concentration-time curve from time zero when first dose is administered to about 24 hours after the first dose is administered (AUC₀₋₂₄) for esomeprazole is about 2134 hr*μg/mL.

In yet another embodiment, the mean area under the plasma concentration-time curve from time zero when first dose is administered to about 24 hours after the first dose is administered (AUC₀₋₂₄) for esomeprazole is about 2000 hr*μg/mL with a % coefficient of variation ranging from 40-80. In yet another embodiment, the mean area under the plasma concentration-time curve from time zero when first dose is administered to about 24 hours after the first dose is administered (AUC₀₋₂₄) for esomeprazole is about 2000 hr*μg/mL.

In still another embodiment, the mean area under the plasma concentration-time curve from time zero when first dose is administered to about 24 hours after the first dose is administered (AUC₀₋₂₄) for esomeprazole is about 1500 hr*μg/mL.

In a still further embodiment, the mean area under the plasma concentration-time curve from time zero when first dose is administered to about 24 hours after the first dose is administered (AUC₀₋₂₄) for esomeprazole is 4911 hr*μg/mL with a % coefficient of variation of 42. In yet still a further embodiment, the mean area under the plasma concentration-time curve from time zero when first dose is administered to about 24 hours after the first dose is administered (AUC₀₋₂₄) for esomeprazole is about 4911 hr*μg/mL.

In one embodiment, the pharmaceutical composition in unit dose form comprises about 500 mg of said naproxen, or pharmaceutically acceptable salt thereof, and about 20 mg of said esomeprazole, or pharmaceutically acceptable salt thereof. In another embodiment, the pharmaceutical composition in unit dose form comprises about 500 mg of said naproxen, or pharmaceutically acceptable salt thereof, and about 30 mg of said esomeprazole, or pharmaceutically acceptable salt thereof.

In yet another embodiment, the unit dose form is administered twice a day for at least 6 days. In still another embodiment, the unit dose form is administered twice a day for at least 9 days.

In still yet another embodiment, the patient in need thereof is an at risk patient. In yet another embodiment, the at risk patient is being treated for a disease or disorder selected from pain and inflammation. In a further embodiment, the at risk patient is being treated for osteoarthritis, rheumatoid arthritis, ankylosing spondylitis, or a combination thereof. In another embodiment, the patient in need thereof is being treated for a disease or disorder selected from pain and inflammation. In yet another embodiment, the patient in need thereof is being treated for osteoarthritis, rheumatoid arthritis, ankylosing spondylitis, or a combination thereof.

In yet another embodiment, the mean Cₘₐₓ for said AM dose of naproxen is 86.2 μg/mL with a % coefficient of variation of 22 and said median Tₘₐₓ is about 3.0 hours; and the mean Cₘₐₓ for said PM dose is 76.8 μg/mL with a % coefficient of variation of 18 and said median Tₘₐₓ is about 10 hours. In another embodiment, the mean Cₘₐₓ for said AM dose of naproxen is about 86.2 μg/mL and said median Tₘₐₓ is about 3.0 hours; and the mean Cₘₐₓ for said PM dose is about 76.8 μg/mL and said median Tₘₐₓ is about 10 hours. In still another embodiment, the mean Cₘₐₓ for said AM dose of naproxen is 80.9 μg/mL with a % coefficient of variation of 23 and said median Tₘₐₓ is about 3.0 hours; and b) the mean Cₘₐₓ for said PM dose is 76.2 μg/mL with a % coefficient of variation of 23 and said median Tₘₐₓ is about 10.4 hours. In still yet another embodiment, the mean Cₘₐₓ for said AM dose of naproxen is about 80.9 μg/mL and said median Tₘₐₓ is about 3.0 hours; and b) the mean Cₘₐₓ for said PM dose is about 76.2 μg/mL and said median Tₘₐₓ is about 10.4 hours.

In yet still another embodiment, the mean area under the plasma concentration-time curve from time zero when the AM dose is administered to about 10 hours after the AM dose is administered (AUC_{0-10,am}) for said AM dose of esomeprazole is 1216 hr*μg/mL with a % coefficient of variation of 69, and the mean area under the plasma concentration-time curve from time zero when the PM dose is administered to about 14 hours after the PM dose is administered (AUC_{0-14,pm}) for said PM dose of esomeprazole is 919 hr*μg/mL with a % coefficient of variation of 84. In a further embodiment, the PM dose has a mean area under the plasma concentration-time curve from time zero when the PM dose is administered to about 14 hours after the PM dose is administered (AUC_{0-14,pm}) is at least 900 hr*μg/mL with a % coefficient of variation ranging from 50-85.

In a still further embodiment, the PM dose has a mean area under the plasma concentration-time curve from time zero when the PM dose is administered to about 14 hours after the PM dose is administered (AUC_{0-14,pm}) of about 900 hr*μg/mL. In a still even further embodiment, the PM dose has a mean area under the plasma concentration-time curve from time zero when the PM dose is administered to about 14 hours after the PM dose is administered (AUC_{0-14,pm}) of about 2100 hr*μg/mL.

In yet another embodiment, the mean area under the plasma concentration-time curve from time zero when the AM dose is administered to about 10 hours after the AM dose is administered (AUC_{0-10,am}) for said AM dose of esomeprazole is about 1216 hr*μg/mL, and the mean area under the plasma concentration-time curve from time zero when the PM dose is administered to about 14 hours after the PM dose is administered (AUC_{0-14,pm}) for said PM dose of esomeprazole is about 919 hr*μg/mL.

In an even further embodiment, the AM dose has a mean area under the plasma concentration-time curve from time zero when the AM dose is administered to about 10 hours after the AM dose is administered (AUC_{0-10,am}) is at least 1200 hr*μg/mL with a % coefficient of variation ranging from 45-70. In a still even further embodiment, the AM dose has a mean area under the plasma concentration-time curve from time zero when the AM dose is administered to about 10 hours after the AM dose is administered (AUC_{0-10,am}) of about 1200 hr*μg/mL.

In a till even further embodiment, the AM dose has a mean area under the plasma concentration-time curve from time zero when the AM dose is administered to about 10 hours after the AM dose is administered (AUC_{0-10,am}) of about 2800 hr*μg/mL.

In still yet a further embodiment, the mean area under the plasma concentration-time curve from time zero when the AM dose is administered to about 10 hours after the AM dose is administered (AUC_{0-10,am}) for said AM dose of esomeprazole is 2779 hr*μg/mL with a % coefficient of variation of 45, and the mean area under the plasma concentration-time curve from time zero when the PM dose is administered to about 14 hours after the PM dose is administered (AUC_{0-14,pm}) for said PM dose of esomeprazole is 2066 hr*μm/mL with a % coefficient of variation of 53. In an even further embodiment, the mean area under the plasma concentration-time curve from time zero when the AM dose is administered to about 10 hours after the AM dose is administered (AUC_{0-10,am}) for said AM dose of esomeprazole is about 2779 hr*μg/mL, and the mean area under the plasma concentration-time curve from time zero when the PM dose is administered to about 14 hours after the PM dose is administered (AUC_{0-14,pm}) for said PM dose of esomeprazole is about 2066 hr*μg/mL.

In an even further embodiment, the pharmaceutical composition in unit dose form is a multilayer tablet comprising at least one core and at least a first layer and a second layer, wherein said core comprises naproxen, or pharmaceutically acceptable salt thereof; said first layer is a coating that at least begins to release the naproxen, or pharmaceutically acceptable salt thereof, when the pH of the surrounding medium of about 3.5 or greater; said second layer is esomeprazole, or pharmaceutically acceptable salt thereof, wherein said esomeprazole, or pharmaceutically acceptable salt thereof, is released at a pH of from about 0 or greater.

In an even further embodiment, esomeprazole, or pharmaceutically acceptable salt thereof, is released at a pH of from 0 or greater. In another embodiment, esomeprazole, or pharmaceutically acceptable salt thereof, is released at a pH of from about 1 or greater. In a further embodiment, esomeprazole, or pharmaceutically acceptable salt thereof, is released at a pH of from 1 or greater. In still another embodiment, esomeprazole, or pharmaceutically acceptable salt thereof, is released at a pH of from about 0 to about 2. In yet a further embodiment, esomeprazole, or pharmaceutically acceptable salt thereof, is released at a pH of from 0 to 2.

In yet still another embodiment, at least a portion of said esomeprazole, or pharmaceutically acceptable salt thereof, is not coated with an enteric coating. In even yet still another embodiment, the first layer is an enteric coating. In an even further embodiment, the pharmaceutical composition in unit dose form is a multilayer tablet comprising a core comprising naproxen, or pharmaceutically acceptable salt thereof, and a first layer comprising a coating that at least begins releasing the naproxen when the pH of the surrounding medium is about 3.5 or greater and a second layer comprising esomeprazole, or pharmaceutically acceptable salt thereof, wherein at least a portion of said esomeprazole, or pharmaceutically acceptable salt thereof, is not surrounded by an enteric coating.

In another embodiment, the first layer is a coating that at least begins to release the naproxen when the pH of the surrounding medium is about 4.0, 4.5, 5.0 or greater. In still yet another embodiment, said first layer begins to release the naproxen when the pH of the surrounding medium is at about 4.0 or greater. In a further embodiment, said first layer begins to release the naproxen when the pH of the surrounding medium is at about 4.5 or greater. In yet a further embodiment, said first layer begins to release the naproxen when the pH of the surrounding medium is at about 5.0 or greater.

In one embodiment, at least about 95% of the esomeprazole, or pharmaceutically acceptable salt thereof, is not surrounded by an enteric coating. In another embodiment, at least about 99% of the esomeprazole, or pharmaceutically acceptable salt thereof, is not surrounded by an enteric coating. In yet another embodiment, at least about 99.5% of the esomeprazole, or pharmaceutically acceptable salt thereof, is not surrounded by an enteric coating.

In yet another embodiment, the multilayer tablet is substantially free of sodium bicarbonate. In still another embodiment, the multilayer tablet is completely (i.e., 100%) free of sodium bicarbonate.

In one embodiment, the dosing regimen is twice a day. In another embodiment, the doses can be separated by a period of at least about 10 hours. In another embodiment, the pharmaceutical composition in unit dose form is given about 1 hour before a patient ingests a meal.

In another embodiment, the pharmaceutical compositions of the present disclosure may be administered therapeutically to patients either short term or over a longer period of time, for example chronically. In other embodiments, long term or chronic administration of the pharmaceutical compositions disclosed herein can result in intragastric pH being at least about 4.0 or greater a higher percentage of time per 24 hour period versus short-term administration. For example, administration of certain pharmaceutical compositions may result in a higher percentage time of intragastric pH being greater than about 4.0 on Day 9 versus Day 1 of treatment.

In another embodiment, the method for delivering a pharmaceutical composition to a patient in need thereof, comprises administering to the patient a pharmaceutical composition in unit dose form comprising naproxen, or pharmaceutically acceptable salt thereof, and esomeprazole, or pharmaceutically acceptable salt thereof, wherein intragastric pH is increased to at least about 4.0 within one hour of administration. In another embodiment, intragastric pH is increased to at least about 4.0 or greater within 30 or 45 minutes of administration.

The pharmaceutical compositions disclosed herein include, but are not limited to, for example, tablets and capsules that can be made in accordance with methods that are standard in the art (see, e.g., Remington's Pharmaceutical Sciences, 16^{th} ed., A Oslo editor, Easton, Pa. (1980)).

Suitable carriers include, but are not limited to: water; salt solutions; alcohols; gum arabic; vegetable oils; benzyl alcohols; polyethylene glycols; gelatin; carbohydrates such as lactose, amylose or starch; magnesium stearate; talc; silicic acid; paraffin; perfume oil; fatty acid esters; hydroxymethylcellulose; polyvinyl pyrrolidone; etc.

The pharmaceutical compositions disclosed herein can be sterilized and, if desired, mixed with, for example, auxiliary agents, such as, for example, preservatives; stabilizers; buffers; coloring agents; and flavoring agents.

In one embodiment, at least one of the layers comprising the pharmaceutical compositions disclosed herein may be applied using standard coating techniques. The layer materials may be dissolved or dispersed in organic or aqueous solvents. The layer materials may include, but are not limited to, for example, one or more of the following materials: methacrylic acid copolymers, shellac, hydroxypropylmethcellulose phthalate, polyvinyl acetate phthalate, hydroxypropylmethyl-cellulose trimellitate, carboxymethylethyl-cellulose, cellulose acetate phthalate, and/or other suitable polymer(s). The pH at which the first layer dissolves can be controlled by the polymer or combination of polymers selected and/or ratio of pendant groups. For example, dissolution characteristics of the polymer film can be altered by the ratio of free carboxyl groups to ester groups. The layers may also contain pharmaceutically acceptable plasticizers, such as, for example, triethyl citrate, dibutyl phthalate, triacetin, polyethylene glycols, polysorbates or other plasticizers. Additives, such as, for example, dispersants, colorants, anti-adhering, and anti-foaming agents may also be used.

In one embodiment, the pharmaceutical compositions disclosed herein can be in the form of a bi- or multi-layer tablet. In a bi-layer tablet, one portion/layer of the tablet contains the esomeprazole, or pharmaceutically acceptable salt thereof, in the required dose along with appropriate excipients, agents to aid dissolution, lubricants, fillers, etc.; and a second portion/layer of the tablet contains the NSAID in the required dose along with other excipients, dissolution agents, lubricants, fillers, etc.

In another embodiment, the naproxen portion/layer is surrounded by a polymeric coating that dissolves at a pH of at least about 3.5 or greater. In yet another embodiment, the naproxen portion/layer is surrounded by a polymeric coating that dissolves at a pH of at least about 4 or greater.

The naproxen, or pharmaceutically acceptable salt thereof, may be granulated by methods such as slugging, low- or high-shear granulation, wet granulation, or fluidized-bed granulation. Of these processes, slugging generally produces tablets of less hardness and greater friability. Low-shear granulation, high-shear granulation, wet granulation and fluidized-bed granulation generally produce harder, less friable tablets.

### EXAMPLES

The disclosure is further defined in the following Example(s). It should be understood the Example(s) are given by way of illustration only. From the above discussion and the Example(s), one skilled in the art can ascertain the essential characteristics of the disclosure, and without departing from the spirit and scope thereof, can make various changes and modifications to adapt the disclosure to various uses and conditions. As a result, the disclosure is not limited by the illustrative example(s) set forth hereinbelow, but rather defined by the claims appended hereto.

### Example 1

### Enteric Coated Naproxen Sodium Core and Famotidine Immediate Release

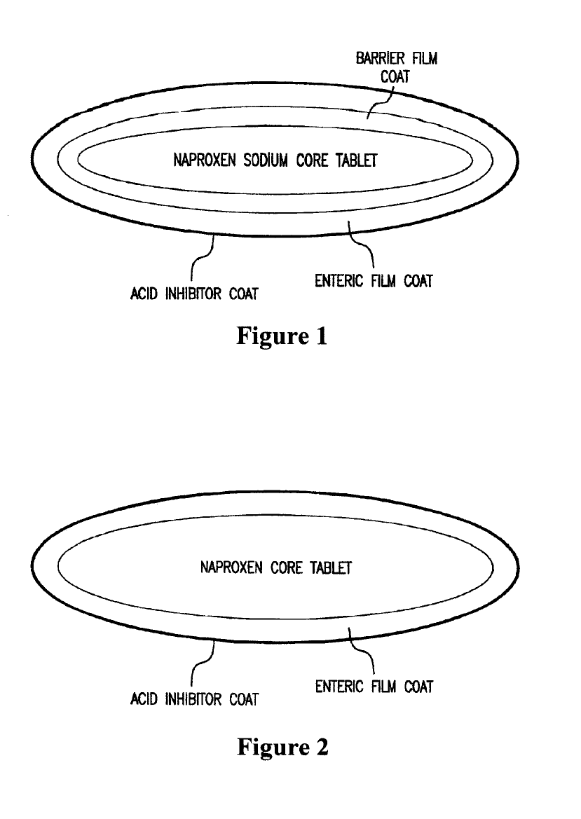

A schematic diagram of a four layer tablet dosage form is shown in FIG. 1. The first layer contains naproxen sodium distributed throughout a matrix of pharmaceutically acceptable fillers, excipients, binding agents, disintegrants, and lubricants.

The second layer is a barrier layer which protects the first layer containing naproxen sodium. The barrier film coat is applied by conventional pan coating technology and the weight of the barrier coat may vary from 1% to 3% of the core tablet weight. In particular embodiments, the core naproxen sodium tablet is coated with coating ingredients such as Opaspray® K-1-4210A or Opadry® YS-1-7006 (Colorcon, West Point, Pa.). Polymer film coating ingredients such as hydroxypropylmethylcellulose 2910 and polyethylene glycol 8000 in a coating suspension may also be used.

The function of the third layer is to prevent the release of naproxen sodium until the dosage form reaches an environment where the pH is above about 4 or 5. The enteric coating does not dissolve in areas of the GI tract where the pH may be below about 4 or 5 such as in an unprotected stomach. Methacrylic acid copolymers are used as the enteric coating ingredient, triethyl citrate and dibutyl phthalate are plasticizers, and ammonium hydroxide is used to adjust the pH of the dispersion. The coating dissolves only when the local pH is above, for example, 5.5 and, as a result, naproxen sodium is released.

The outermost layer contains an “acid inhibitor” in an effective amount which is released from the dosage form immediately after administration to the patient. The acid inhibitor in the present example is a proton pump inhibitor or, preferably the H2 blocker famotidine, which raises the pH of the gastrointestinal tract to above 4. The typical effective amount of famotidine in the dosage form will vary from 5 mg to 100 mg. A typical film coating formulation contains Opadry Clear® YS-1-7006 which helps in the formation of the film and in uniformly distributing famotidine within the fourth layer without tablets sticking to the coating pan or to each other during application of the film coat. Other ingredients may include: plasticizers such as triethyl citrate, dibutyl phthalate, and polyethylene glycol; anti-adhering agents such as talc; lubricating ingredients such as magnesium stearate; and opacifiers such as titanium dioxide. In addition, the pH of the film coating solution can be adjusted to aid in dissolution of the famotidine. The film coating is thin and rapidly releases famotidine for absorption.

| Core Tablet Ingredients | % W/W | mg/Tablet |
|---|---|---|
| Naproxen sodium, USP | 74.074 | 500.00 |
| Microcrystalline cellulose, NF | 17.166 | 115.87 |
| (Avicel PH 200) | | |
| Povidone (K29/32), USP | 3.450 | 23.29 |
| Talc, USP | 4.350 | 29.36 |
| Magnesium Stearate, NF | 0.960 | 6.48 |
| Total | 100.00 | 675.00 |

| Barrier Film Coating Ingredients | % W/W |
|---|---|
| Opadry Clear ® YS-1-7006 | 5.00 |
| Purified water USP | 95.00 |
| Total | 100.00 |

Enteric Coating Dispersion

| Ingredients | % W/W |
|---|---|
| Methacrylic Acid Copolymer, NF | 7.30 |
| (Eudragit L-100-55) | |
| Methacrylic Acid Copolymer, NF | 7.30 |
| (Eudragit L-100) | |
| Triethyl Citrate, NF | 2.95 |
| Dibutyl Phthalate, NF | 1.17 |
| Ammonium Hydroxide (30%), NF | 0.87 |
| Purified water, USP | 80.41 |
| Total | 100.00 |

Famotidine Coating Dispersion

| Ingredients | % W/W |
|---|---|
| Famotidine, USP | 3.0 |
| Opadry Clear ® (YS-1-7006) | 5.0 |
| Talc, USP | 3.0 |
| Purified Water, USP | 89.0 |
| Total | 100.0 |

### Example 2

### Enteric Coated Naproxen Core and Famotidine Immediate Release

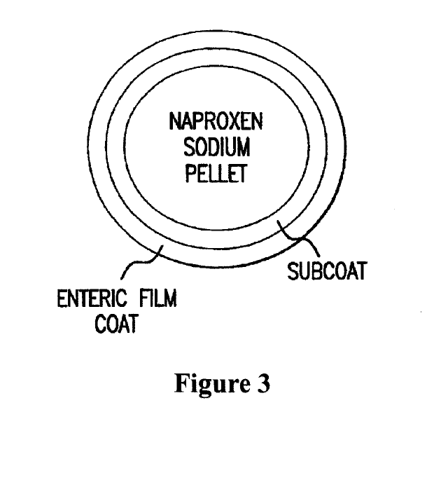

FIG. 2 illustrates a three layered dosage form which releases famotidine immediately after ingestion by the patient in order to raise the pH of the gastrointestinal tract to above about 4. The innermost layer contains naproxen uniformly distributed throughout a matrix of pharmaceutically acceptable excipients. These excipients perform specific functions and may serve as binders, disintegrants, or lubricants. A pharmaceutically acceptable enteric coating surrounds the naproxen core. The function of the enteric coat is to delay the release of naproxen until the dosage form reaches an environment where the pH is above about 4. The coating does not dissolve in the harshly acidic pH of the unprotected stomach. It contains methacrylic acid copolymers which prevent the release of naproxen in the unprotected stomach. Also included are: triethyl citrate, a plasticizer; simethicone emulsion, an anti-foaming agent; and sodium hydroxide which is used to adjust the pH of the dispersion.

The outermost layer contains an “acid inhibitor” in an effective amount which is released from the dosage form immediately after administration to the patient. The acid inhibitor in this example is a proton pump inhibitor or, preferably, the H2 blocker famotidine which raises the pH of the stomach to above 4. A typical film coating formulation contains Opadry Clear® YS-1-7006 which helps in the formation of the film and in uniformly distributing famotidine in the fourth layer without tablets sticking to the coating pan or sticking to each other during application of the film coat. Other ingredients are: plasticizers such as polyethylene glycol 8000; anti-adhering agents such as talc; lubricating ingredients such as magnesium stearate; and opacifiers such as titanium dioxide. In addition, the pH of the film coating solution can be adjusted to aid in dissolution of the famotidine. The film coating is thin and rapidly releases famotidine for absorption.

| Core Tablet Ingredients | % W/W | mg/Tablet |
|---|---|---|
| Naproxen, USP | 90.91 | 500.00 |
| Povidone K-90, USP | 2.00 | 11.00 |
| Starch, USP | 2.59 | 14.25 |
| Croscarmellose Sodium, USP | 4.00 | 22.00 |
| Magnesium Stearate, NF | 0.50 | 2.75 |
| Total | 100.00 | 550.00 |
| Purified Water, USP qs | | |

| Enteric Coating Dispersion Ingredients | % W/W |
|---|---|
| Methacrylic Acid Copolymer Type C, NF | 14.5 |
| (Eudragit L-100-55) | |
| Talc, USP | 3.8 |
| Sodium Hydroxide, NF | 0.2 |
| Triethyl Citrate, NF | 1.7 |
| Simethicone Emulsion, USP | 0.02 |
| Purified Water, USP | 79.78 |
| Total | 100.00 |

Famotidine Coating Dispersion

| Ingredients | % W/W |
|---|---|
| Famotidine, USP | 3.0 |
| Opadry Clear ® (YS-1-7006) | 5.0 |
| Talc, USP | 3.0 |
| Purified Water, USP | 89.0 |
| Total | 100.0 |

### Example 3

### Naproxen Controlled Release Core and Famotidine Immediate Release

A trilayer tablet which separates famotidine contained in the film coat from controlled-release naproxen may be used in the present invention. The core tablet of naproxen is formulated using excipients which control the drug release for therapeutic relief from pain and inflammation for 24 hours. FIG. 2 shows an example of an appropriate trilayer tablet. In this particular example, naproxen is mixed with a polymeric material, hydroxypropyl-methylcellulose and granulated with water. The granules are dried, milled, and blended with a lubricant, such as magnesium stearate. They are then compacted into tablets.

The controlled-release core tablet of naproxen is film coated with a pharmaceutically acceptable enteric coating. The function of the enteric coat is to delay the release of naproxen until the dosage form reaches an environment where the pH is above about 4. The coating does not dissolve in the extremely acidic pH of the unprotected stomach. The function of methacrylic acid copolymers is to prevent the release of naproxen until the pH of the stomach rises. Triethyl citrate is a plasticizer, simethicone emulsion is a anti-foaming agent, and sodium hydroxide is used to adjust the pH of the dispersion.

The outermost layer contains an “acid inhibitor” which is released from the dosage form immediately after administration to the patient. The acid inhibitor in the present example is a proton pump inhibitor or, preferably, the H2 blocker famotidine which consistently raises the pH of the stomach to above 4. The typical effective amount of famotidine in the dosage will vary from 5 mg to 100 mg. A typical film coating formulation contains Opadry Blue® YS-1-4215 which is essential for film formation and for the uniform application of famotidine to the core tablet. Polymer film coating ingredients, hydroxypropylmethylcellulose or Opaspray® K-1-4210A (Colorcon, West Point, Pa.) may also be used. Other ingredients which help in the formation of the film and in the uniform application of famotidine to the core tablet are: plasticizers such as triethyl citrate and dibutyl phthalate; anti-adhering agents such as talc; lubricating ingredients such as magnesium stearate; and opacifiers such as titanium dioxide. In addition, the pH of the film coating solution can be adjusted to aid in dissolution of the famotidine. The film coating is thin and rapidly releases famotidine for absorption.

| Core Tablet Ingredients | % W/W | mg/Tablet |
|---|---|---|
| Naproxen, USP | 94.00 | 750 |
| Hydroxypropyl methylcellulose | 5.00 | 39.9 |
| 2208, USP (viscosity 15000 cps) | | |
| Magnesium Stearate, NF | 1.00 | 7.95 |
| Total | 100.00 | 797.85 |

| Enteric Coating Dispersion Ingredients | % W/W |
|---|---|
| Methacrylic Acid Copolymer Type C, NF | 14.5 |
| (Eudragit L-100-55) | |
| Talc, USP | 3.8 |
| Sodium Hydroxide, NF | 0.2 |
| Triethyl Citrate, NF | 1.7 |
| Simethicone Emulsion, USP | 0.02 |
| Purified Water, USP | 79.78 |
| Total | 100.00 |

| Famotidine Coating Dispersion Ingredients | % W/W |
|---|---|
| Famotidine, USP | 2.0 |
| Opadry Blue ® (YS-1-4215) | 10.0 |
| Talc, USP | 9.0 |
| Purified Water, USP | 79.0 |
| Total | 100.0 |

### Example 4

### Naproxen and Famotidine Controlled Release Core and Famotidine Immediate Release

A trilayer tablet which separates famotidine contained in the film coat from controlled-release naproxen and famotidine may be used in the present invention. The core tablet of naproxen and famotidine is formulated using excipients which control the drug release for therapeutic relief from pain and inflammation for 24 hours. FIG. 2 is an example of an appropriate trilayer tablet. In this particular example, naproxen and famotidine are mixed with a polymeric material, hydroxypropylmethylcellulose and granulated with water. The granules are dried, milled, and blended with a lubricant, such as magnesium stearate. They are then compacted into tablets.

The controlled-release core tablet of naproxen and famotidine is film coated with a pharmaceutically acceptable enteric coating. The function of the enteric coat is to delay the release of naproxen until the dosage form reaches an environment where the pH is above about 4. The coating does not dissolve in the extremely acidic pH of the unprotected stomach. The function of methacrylic acid copolymers is to prevent the release of naproxen until the pH of the stomach rises. Triethyl citrate is a plasticizer, simethicone emulsion is a anti-foaming agent, and sodium hydroxide is used to adjust the pH of the dispersion

The outermost later contains an “acid inhibitor” which is released from the dosage form immediately after administration to the patient. The acid inhibitor in the present example is a proton pump inhibitor or, preferably, the H2 blocker famotidine which consistently raises the pH of the stomach to above 4. The typical effective amount of famotidine in the dosage will vary from 5 mg to 100 mg. A typical film coating formulation contains Opadry Blue® YS-1-4215 which is essential for film formation and for the uniform application of famotidine to the core tablet. Polymer film coating ingredients, hydroxypropylmethylcellulose or Opaspray® K-1-4210A (Colorcon, West Point, Pa.) may also be used. Other ingredients which help in the formation of the film and in the uniform application of famotidine to the core tablet are: plasticizers such as triethyl citrate and dibutyl phthalate; anti-adhering agents such as talc; lubricating ingredients such as magnesium stearate; and opacifiers such as titanium dioxide. In addition, the pH of the film coating solution can be adjusted to aid in dissolution of the famotidine. The film coating is thin and rapidly releases famotidine for absorption.

| Core Tablet Ingredients | % W/W | mg/Tablet |
|---|---|---|
| Naproxen, USP | 88.05 | 500 |
| Famotidine, USP | 3.52 | 20.0 |
| Hydroxypropyl methylcellulose | 7.03 | 39.9 |
| 2208, USP (viscosity 15000 cps) | | |
| Magnesium Stearate, NF | 1.40 | 7.95 |
| Total | 100.00 | 567.85 |

| Enteric Coating Dispersion Ingredients | % W/W |
|---|---|
| Methacrylic Acid Copolymer Type C, NF | 14.5 |
| (Eudragit L-100-55) | |
| Talc, USP | 3.8 |
| Sodium Hydroxide, NF | 0.2 |
| Triethyl Citrate, NF | 1.7 |
| Simethicone Emulsion, USP | 0.02 |
| Purified Water, USP | 79.78 |
| Total | 100.00 |

Famotidine Coating Dispersion

| Ingredients | % W/W |
|---|---|
| Famotidine, USP | 2.0 |
| Opadry Blue ® (YS-1-4215) | 10.0 |
| Talc, USP | 9.0 |
| Purified Water, USP | 79.0 |
| Total | 100.0 |

### Example 5

### Enteric Coated Naproxen Sodium Core and Pantoprazole Immediate Release in Film Coat

A schematic diagram of a four layer tablet dosage form is shown in FIG. 1. The first layer contains naproxen sodium distributed throughout a matrix of pharmaceutically acceptable fillers, excipients, binding agents, disintegrants, and lubricants.

The second layer is a barrier layer which protects the first layer containing naproxen sodium. The barrier film coat is applied by conventional pan coating technology and the weight of the barrier coat may vary from 1% to 3% of the core tablet weight. In particular embodiments, the core naproxen sodium tablet is coated with coating ingredients such as Opaspray® K-1-4210A or Opadry® YS-1-7006 (Colorcon, West Point, Pa.). Polymer film coating ingredients such as hydroxypropylmethylcellulose 2910 and polyethylene glycol 8000 in a coating suspension may also be used.

The third layer is an enteric film coat. It does not dissolve in areas of the GI tract where the pH may be below 4 such as in an unprotected stomach but it dissolves only when the local pH is above about 4. Therefore, the function of the third layer is to prevent the release of naproxen sodium until the dosage form reaches an environment where the pH is above 4. In this example, hydroxypropylmethylcellulose phthalate is the enteric coating ingredient, cetyl alcohol is a plasticizer and acetone and alcohol are solvents.

The fourth layer contains an “acid inhibitor” in an effective amount which is released from the dosage form as soon as the film coat dissolves. The acid inhibitor in this example is a proton pump inhibitor, pantoprazole, which raises the pH of the gastrointestinal tract to above 4. The typical effective amount of pantoprazole in the dosage form may vary from 10 mg to 200 mg. The film coat is applied by conventional pan coating technology and the weight of film coat may vary from 4% to 8% of the core tablet weight. Other ingredients are, plasticizers such as triethyl citrate, dibutyl phthalate, anti-adhering agents such as talc, lubricating ingredients such as magnesium stearate, opacifiers such as, titanium dioxide, and ammonium hydroxide to adjust the pH of the dispersion. The film coating is thin and rapidly releases pantoprazole for absorption. Therefore, pantoprazole releases first and then the core erodes and releases naproxen sodium.

| Core Tablet Ingredients | % W/W | mg/tablet |
|---|---|---|
| Naproxen sodium, USP | 74.075 | 500.00 |
| Microcrystalline cellulose, NF | 17.165 | 115.87 |
| (Avicel PH 200) | | |
| Povidone (K29/32), USP | 3.450 | 23.29 |
| Talc, USP | 4.350 | 29.36 |
| Magnesium Stearate, NF | 0.960 | 6.48 |
| Total | 100.00 | 675.00 |

Naproxen sodium, 50% microcrystalline cellulose and povidone are dry mixed and wet granulated in an appropriate granulator with sufficient purified water. The wet granules are dried, milled, and blended with the remaining 50% microcrystalline cellulose, talc and magnesium stearate. The final granule blend is compressed into tablets.

| Barrier Film Coating Ingredients | % W/W |
|---|---|
| Opadry ® Clear YS-1-7006 | 5.00 |
| Purified Water, USP | 95.00 |
| Total | 100.00 |

Opadry clear is added slowly to purified water and mixing is continued until Opadry is fully dispersed. The solution is sprayed on to the tablet cores in a conventional coating pan until proper amount of Opadry clear is deposited on the tablets.

| Enteric Coating Ingredients | % W/W |
|---|---|
| Hydroxypropyl methylcellulose phthalate, NF | 5.5 |
| Cetyl alcohol, NF | 0.3 |
| Acetone, NF | 66.3 |
| Alcohol, USP | 27.9 |
| Total | 100.00 |

Hydroxypropylmethylcellulose phthalate and cetyl alcohol are dissolved in a mixture of alcohol and acetone. The solution is then sprayed on to the tablet bed in proper coating equipment. A sample of the tablets is tested for gastric resistance and the coating stopped if the tablets pass the test.

| Pantoprazole Film Coating Ingredients | % W/W |
|---|---|
| Pantoprazole sodium, USP | 5.00 |
| Opadry ® Clear YS-1-7006 | 5.00 |
| Sodium carbonate, NF | 1.20 |
| Purified Water, USP | 88.80 |
| Total | 100.00 |

Pantoprazole sodium is dissolved in purified water containing sodium carbonate in solution. After thorough mixing, Opadry clear is added slowly and mixing is continued until Opadry is fully dispersed. The suspension is sprayed on to the tablet cores in a conventional coating pan until the proper amount of pantoprazole sodium is deposited.

### Example 6

### Enteric Coated Naproxen Sodium Core and Omeprazole Immediate Release in Film Coat

A schematic diagram of a four layer tablet dosage form is shown in FIG. 1. The first layer contains naproxen sodium distributed throughout a matrix of pharmaceutically acceptable fillers, excipients, binding agents, disintegrants, and lubricants.

The second layer is a barrier layer which protects the first layer containing naproxen sodium. The barrier film coat is applied by conventional pan coating technology and the weight of the barrier coat may vary from 1% to 3% of the core tablet weight. In particular embodiments, the core naproxen sodium tablet is coated with coating ingredients such as Opaspray® K-1-4210A or Opadry® YS-1-7006 (Colorcon, West Point, Pa.). Polymer film coating ingredients such as hydroxypropylmethylcellulose 2910 and polyethylene glycol 8000 in a coating suspension may also be used.

The third layer is an enteric film coat. It does not dissolve in areas of the GI tract where the pH is below 4 such as in an unprotected stomach but it dissolves only when the local pH is above 4. Therefore, the function of the third layer is to prevent the release of naproxen sodium until the dosage form reaches an environment where the pH is above about 4. In this example, hydroxypropylmethylcellulose phthalate is the enteric coating ingredient, cetyl alcohol is a plasticizer and acetone and alcohol are solvents.

The fourth layer contains an “acid inhibitor” in an effective amount which is released from the dosage form as soon as the film coat dissolves. The acid inhibitor in this example is a proton pump inhibitor, omeprazole, which raises the pH of the gastrointestinal tract to above 4. The typical effective amount of omeprazole in the dosage form may vary from 5 mg to 50 mg. The film coat is applied by conventional pan coating technology and the weight of film coat may vary from 4% to 8% of the core tablet weight. Other ingredients are, plasticizers such as triethyl citrate, dibutyl phthalate, anti-adhering agents such as talc, lubricating ingredients such as magnesium stearate, opacifiers such as, titanium dioxide, and ammonium hydroxide to adjust the pH of the dispersion. The film coating is thin and rapidly releases omeprazole for absorption. Therefore, omeprazole is released first and then the core erodes and releases naproxen sodium.

| Core Tablet Ingredients | % W/W | mg/tablet |
|---|---|---|
| Naproxen sodium, USP | 74.075 | 500.00 |
| Microcrystalline cellulose, NF | 17.165 | 115.87 |
| (Avicel PH 200) | | |
| Povidone (K29/32), USP | 3.450 | 23.29 |
| Talc, USP | 4.350 | 29.36 |
| Magnesium Stearate, NF | 0.960 | 6.48 |
| Total | 100.00 | 675.00 |

Naproxen sodium, 50% microcrystalline cellulose and povidone are dry mixed and wet granulated in an appropriate granulator with sufficient purified water. The wet granules are dried, milled, and blended with the remaining 50% microcrystalline cellulose, talc and magnesium stearate. The final granule blend is compressed into tablets.

| Barrier Film Coating Ingredients | % W/W |
|---|---|
| Opadry ® Clear YS-1-7006 | 5.00 |
| Purified Water, USP | 95.00 |
| Total | 100.00 |

Opadry clear is added slowly to purified water and mixing is continued until Opadry is fully dispersed. The solution is sprayed on to the tablet cores in a conventional coating pan until the proper amount of Opadry clear is deposited on the tablets.

| Enteric Coating Ingredients | % W/W |
|---|---|
| Methacrylic Acid Copolymer, NF | 6.0 |
| (Eudragit L-100-55) | |
| Triethyl Citrate, NF | 0.6 |
| Talc, USP | 3.0 |
| Purified Water, USP | 5.0 |
| Isopropyl Alcohol, USP | 85.40 |
| Total | 100.00 |

Methacrylic acid copolymer, triethyl citrate, and talc are dissolved in a mixture of isopropyl alcohol and water. The solution is then sprayed on to the tablet bed in a proper coating equipment. A sample of the tablets is tested for gastric resistance and the coating is stopped if the tablets pass the test.

| Omeprazole Film Coating Ingredients | % W/W |
|---|---|
| Omeprazole, USP | 5.00 |
| Opadry ® Clear YS-1-7006 | 5.00 |
| Purified Water, USP | 10.00 |
| Isopropyl Alcohol, USP | 80.00 |
| Total | 100.00 |

Omeprazole is dissolved in a purified water and isopropyl alcohol mixture. After thorough mixing, Opadry clear is added slowly and mixing is continued until Opadry is fully dispersed. The suspension is sprayed on to the tablet cores in a conventional coating pan until proper amount of omeprazole is deposited on the tablets.

### Example 7

### Naproxen Sodium Delayed Release and Omeprazole Immediate Release Capsule

A coordinated delivery dosage may be used to provide fast release of an acid inhibitor, a proton pump inhibitor, omeprazole which raises the pH of the gastrointestinal tract to above 4, and the delayed release of a non-steroidal anti-inflammatory drug, naproxen sodium. Omeprazole granules modify the pH of the stomach such that the drug readily dissolves and is absorbed in the stomach without significant degradation. The typical effective amount of omeprazole in the dosage form may vary from 5 mg to 50 mg. The release of naproxen sodium is delayed by enteric coating.

Omeprazole granules contain an alkalizing excipient such as sodium bicarbonate. Other soluble alkalizing agents such as potassium bicarbonate, sodium carbonate, sodium hydroxide, or their combinations may also be used. The alkalizing agent helps solubilize and protect omeprazole from degradation before its absorption. Sodium lauryl sulfate helps in the wetting of omeprazole. Other surfactants may be used to perform the same function. In the present example, hydroxypropyl methylcellulose helps in granule formation, sodium starch glycolate is a disintegrant, and magnesium stearate is a lubricant. Other excipients may also be used to perform these functions.

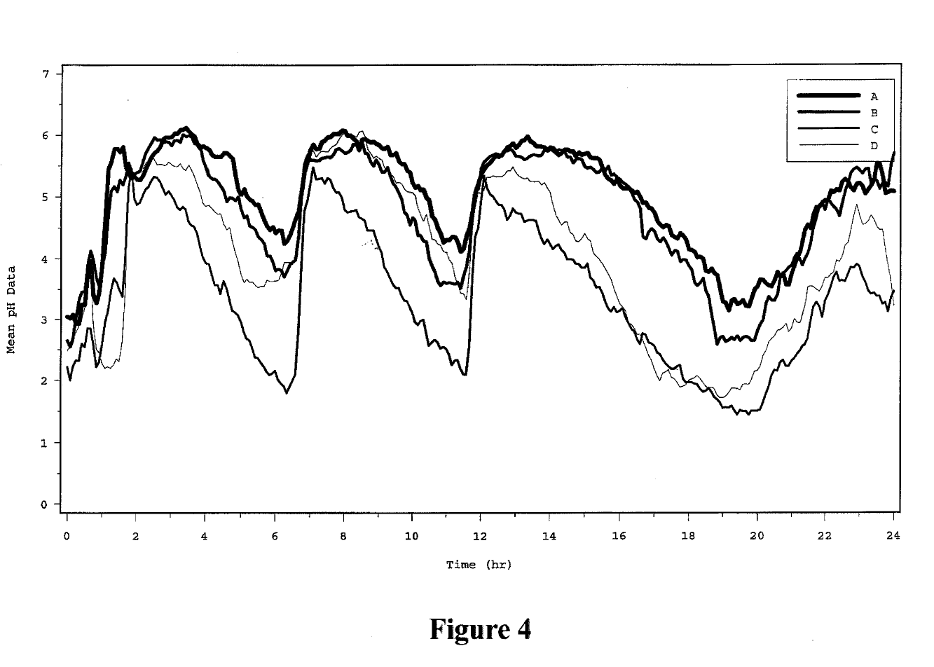

Naproxen sodium pellets as shown in FIG. 3 are prepared by the wet massing technique and the conventional extrusion and spheronization process. The excipients used in the formulation are microcrystalline cellulose, and povidone. The pellets after drying and classification are coated with a protective subcoating containing povidone. Other coating ingredients may also be used such as Opaspray K-1-4210A or Opadry YS-1-7006 (trademarks of Colorcon, West Point, Pa.). Polymer film coating ingredients such as hydroxypropylmethylcellulose 2910 and polyethylene glycol 8000 in a subcoating suspension are also alternatives. Other ingredients are, plasticizers such as triethyl citrate, dibutyl phthalate, anti-adhering agents such as talc, lubricating ingredients such as magnesium stearate, opacifiers such as, titanium dioxide.

The subcoated pellets are enteric coated using enteric coating polymers. In this example, the enteric coating polymer is methacrylic acid copolymer and the plasticizer is dibutyl phthalate which are dissolved in a mixture of acetone and alcohol. The enteric film does not dissolve in the acidic pH but dissolves when the pH in the gut is above about pH 6 and releases naproxen sodium.

| Omeprazole Granules | % W/W | mg/capsule |
|---|---|---|
| Omeprazole, USP | 12.9 | 20.00 |
| Sodium Bicarbonate, USP | 82.40 | 127.72 |
| Hydroxypropyl methylcellulose, USP | 2.00 | 3.10 |
| Sodium lauryl sulfate, NF | 0.20 | 0.31 |
| Sodium starch glycolate, NF | 2.00 | 3.10 |
| Magnesium stearate, NF | 0.50 | 0.77 |
| Total | 100 | 100 |

Hydroxypropylmethylcellulose is dissolved in water, then sodium lauryl sulfate is added and the solution is mixed. Omeprazole, microcrystalline cellulose, and sodium bicarbonate are dry mixed together and granulated with the granulating solution. The granulation is mixed until proper granule formation is reached. The granulation is then dried, milled, and blended with magnesium stearate.

| Pellet Ingredients | % W/W | mg/tablet |
|---|---|---|
| Naproxen sodium, USP | 86.80 | 250.00 |
| Microcrystalline cellulose, NF | 11.10 | 32.00 |
| (Avicel PH 200) | | |
| Povidone (K90), USP | 2.10 | 6.00 |
| Total | 100.00 | 288.00 |

Povidone is dissolved in water. Naproxen sodium and microcrystalline cellulose are dry mixed and granulated with povidone solution. The wet mass is mixed until proper consistency is reached. The wet mass is then pressed through an extruder and spheronized to form pellets. The pellets are then dried and classified into suitable particle size range.

| Subcoat Ingredients | % W/W |
|---|---|
| Povidone (K29-32), USP | 10.00 |
| Alcohol, USP | 90.00 |
| Total | 100.00 |

The pellet cores are coated using povidone solution by a conventional coating pan method to a weight gain of 1-2%.

| Enteric Coating Ingredients | % W/W |
|---|---|
| Methacrylic Acid Copolymer, NF | 8.20 |
| (Eudragit L-100) | |
| Diethyl Phthalate, NF | 1.70 |
| Acetone, NF | 33.30 |
| Isopropyl Alcohol, USP | 56.80 |
| Total | 100.0 |

Eudragit L-100 is dissolved in isopropanol and acetone and diethyl phthalate is dissolved. The solution is sprayed on the pellet cores using proper film coating equipment. A sample of the pellets is tested for gastric resistance before stopping the coating process.

Omeprazole fast release granules and naproxen sodium delayed release pellets are blended together and filled into appropriate size capsules to contain 250 mg naproxen sodium and 20 mg omeprazole per capsule.

### Example 8

### Naproxen Delayed Release and Omeprazole Immediate Release Capsule

The present Example is directed to a coordinated delivery dosage form containing omeprazole and naproxen. The formulation contains 10 mg omeprazole and uses methylcellulose as a binder and croscarmellose sodium as a disintegrant. Naproxen pellets as shown in FIG. 3 do not need a subcoating layer and are enteric coated with an aqueous dispersion of methacrylic acid copolymer. Optionally, these pellets could be compressed into a core and film coated with an acid inhibitor and thereby form a bilayer tablet.

| Omeprazole Granules | % W/W | mg/capsule |
|---|---|---|
| Omeprazole, USP | 6.45 | 10.00 |
| Sodium Bicarbonate, USP | 88.85 | 137.71 |
| Methylcellulose, USP | 2.00 | 3.10 |
| Sodium lauryl sulfate, NF | 0.20 | 0.31 |
| Croscarmellose sodium, NF | 2.00 | 3.10 |
| Magnesium stearate, NF | 0.50 | 0.78 |
| Total | 100 | 100 |

Methylcellulose is dissolved in water, then sodium lauryl sulfate is added to the solution and mixed. Omeprazole, microcrystalline cellulose, and sodium bicarbonate are dry mixed together and granulated with the granulating solution. The granulation is mixed until proper granule formation is reached. The granulation is then dried, milled, and blended with magnesium stearate.

| Pellet Ingredients | % W/W | mg/tablet |
|---|---|---|
| Naproxen, USP | 76.22 | 250.00 |
| Microcrystalline cellulose, NF | 21.78 | 71.44 |
| (Avicel PH 200) | | |
| Povidone (K90), USP | 2.00 | 6.56 |
| Total | 100.00 | 328.00 |

Povidone is dissolved in water. Naproxen and microcrystalline cellulose are dry mixed and granulated with povidone solution. The wet mass is mixed until proper consistency is reached. The wet mass is then pressed through an extruder and spheronized to form pellets. The pellets are then dried and classified into a suitable particle size range.

| Enteric Coating Ingredients | % W/W |
|---|---|
| Methacrylic Acid Copolymer, NF (Eudragit | 15.60 |
| L30D 30% dispersion) | |
| Talc, USP | 7.60 |
| Triethyl citrate, NF | 1.60 |
| Simethicone Emulsion, USP | 0.20 |
| (Silicone antifoam emulsion SE 2) | |
| Purified Water, USP | 74.80 |

Eudragit 30D is dispersed in purified water and simethicone emulsion. Talc and triethyl citrate are then dispersed. The suspension is sprayed on the pellet cores using proper film coating equipment. A sample of the pellets is tested for gastric resistance before stopping the coating process. Omeprazole fast release granules and naproxen sodium delayed release pellets are blended together and filled into appropriate size capsules to contain 250 mg naproxen and 10 mg omeprazole per capsule.

### Example 9

### Clinical Study of the Relationship of Gastric pH to NSAID-Induced Gastric Ulcers

Sixty-two subjects were enrolled in a clinical study and randomly assigned to three groups. The following three groups were administered study medication twice daily for five days: (a) 550 mg naproxen sodium (n=10), (b) 40 mg famotidine given with 550 mg of naproxen or famotidine followed 90 minutes later by 550 mg naproxen, (n=39) or (c) 20 mg omeprazole followed by 550 mg naproxen sodium (n=13). Gastric pH was measured hourly beginning at the time of dosing of the final daily dose of study medication and for 8-10 hours thereafter. Subjects had a gastric endoscopy performed at the beginning and on Day 5 prior to the morning dose of study medication to identify gastric and duodenal irritation; no subjects were admitted to the study if gastric irritation was present at the time of initial endoscopy.

Five patients, three (33%) in the naproxen alone group and two (5%) in the famotidine/naproxen group, presented with gastroduodenal ulcers at the end of the study. In the naproxen alone group, the pH was greater than 4 only 4% of the time, and in the famotidine/naproxen group the pH was greater than 4 forty-nine percent of the time during the 8-10 hours following naproxen sodium dosing. Additionally, Lanza grade 3 or 4 damage was present in 28% (n=11) of the subjects receiving famotidine/naproxen sodium, and present 100% (n=10) in the naproxen sodium treatment group. Monitoring of gastric acidity on day 5 indicated that patients with Lanza scores of greater than 2 had integrated gastric acidity of greater than 100 mmol-hr·/L. Only 20-40% of patients with integrated gastric acidity of less than 100 mmol-hr/L had gastric pathology, whereas all patients with integrated gastric acidity greater than 100 mmol-hr/L had pathology.

### Example 10

### Famotidine and Enteric Coated Naproxen Reduce Gastroduodenal Damage Due to NSAID Therapy

Thirty-seven patients were randomized to two groups for a one week study of twice-daily dosing of: 500 mg enteric coated naproxen, and 500 mg enteric coated naproxen preceded by 40 mg famotidine. Endoscopies were conducted on all patients prior to first dosing and on the final day of the study. No subjects had evidence of gastroduodenal damage at the beginning of the study (at first endoscopy).

At the second endoscopy, Lanza scores for gastroduodenal damage were assessed for all subjects. 39% of the subjects in the enteric coated naproxen 500 mg group had grade 3-4 gastroduodenal damage. This is lower than the percentage that would be expected for the administration of 500 mg of non-enteric naproxen based upon previous work. Nevertheless, subjects administered 500 mg enteric coated naproxen and 40 mg famotidine had an even lower incidence of grade 3-4 gastroduodenal damage (26%) than subjects who had previously taken enteric coated naproxen alone which demonstrates the value of combining acid inhibition with enteric coating of NSAID to minimize the gastrointestinal damage.

### Example 11

### Clinical Study of the Pharmacodynamic Measurements of Intragastric pH

A randomized, open-label, 4-way cross-over study to evaluate the effect on days 1 and 9 of twice daily oral administration of three PN 400 formulations (enteric coated naproxen 500 mg combined with non-enteric coated esomeprazole 10, 20, or 30 mg) versus the effect of twice daily oral administration of a separate 500 mg non-enteric coated naproxen tablet and once daily oral administration of a separate EC esomeprazole (20 mg) capsule (Nexium® 20 mg capsule) on the 24-hour intragastric pH and pharmacokinetic parameters (i.e., Cₘₐₓ, Tₘₐₓ, AUC_{0-10,am}, AUC_{0-14,pm}, AUC₀₋₂₄, AUC_{0-t,am}, and AUC_{0-t,pm}) of healthy volunteers. The PN400 tablet is a multilayer tablet comprising an inner core of naproxen surrounded by a first layer comprising an enteric coating and a second layer comprising non-enteric coated esomeprazole.

The study was designed to compare the pharmacodynamic (PD) measurements of intragastric pH (percent time of pH>4.0) on Day 9 of three PN 400 formulations following twice daily (BID) administration versus a combination of non-enteric coated naproxen taken BID and EC esomeprazole (20 mg) taken once daily.

The study was also designed to compare the PD measurement of intragastric pH (percent time of pH>4.0) on Day 1 of three PN 400 formulations following BID administration versus a combination of non-enteric coated naproxen taken BID and EC esomeprazole taken once daily; and assess the pharmacokinetics of esomeprazole and naproxen on Day 1 and Day 9 in each of the treatment groups.

This was a single-center study in 28 healthy adults. The study consisted of four 9-day treatment periods. The first, second and third treatment periods were followed by a washout period of at least 12 days. Eligible subjects reported to the Phase 1 unit in the PM of Day 0 for screening.

Screening procedures included procurement of informed consent, medical and drug history, physical examination, vital signs, 12-lead electrocardiogram (ECG), clinical laboratory testing, urine drug screen, pregnancy test for females of childbearing potential, and helicobacter pylori breath test. Clinical laboratory tests, physical examination, and measurement of vital signs were performed at Screening and the Final Visit. A 12-lead electrocardiogram (ECG) and 13C-urea breath test to screen for possible helicobacter pylori infection were performed at Screening. A urine drug screen for all subjects and a urine pregnancy test for women of childbearing potential were performed on Days 0 and 8 of each treatment period. On Days 1 and 9 of each treatment period, 24-hour blood sampling was performed for pharmacokinetic (PK) assessments. Subjects were instructed to report to the Phase 1 unit within 14 days of the initiation of screening procedures.

Twenty-eight subjects were planned, randomized and treated, and data for 25 subjects were analyzed as the Per-Protocol (PP) population; the Intent-to-Treat (ITT), Safety and PK populations included all 28 subjects. Subjects were healthy males or non-lactating, non-pregnant females 18 to 55 years of age with a body mass index of 19-32 kg/m², were helicobacter pylori (H. pylori) negative, and were generally in good health with no history of peptic ulcer disease or other acid-related gastrointestinal (GI) symptoms.

Subject enrollment and disposition are summarized in Table 2. Twenty-eight subjects were randomized and treated at one investigational center; these subjects comprised the PK population. One subject dropped out of the study for personal reasons after completing 3 treatment periods (Treatments A [PN 400/E30], B [PN 400/E20] and D [EC E20+naproxen]). The PP population included 25 subjects.

TABLE 2
Subject Enrollment and Disposition - All Subjects

| | Subjects (%) |
|---|---|
| | N = 28 |
| Subjects Randomized and Treated | 28 (100) |
| Safety Population | 28 (100) |
| Intent-to-Treat Population | 28 (100) |
| Per Protocol Population | 25 (89) |
| PK Population | 28 (100) |
| Subjects Completed | 27 (96) |
| Subjects Withdrawn Prematurely | 1 (4) |
| Adverse event | 0 |
| Withdrew consent | 0 |
| Lost to follow-up | 0 |
| Other | 1 (4) |

The demographic characteristics of the ITT population at Screening are summarized in Table 3. The study population was 68% male and had a mean age of approximately 25 years. All subjects were white and non-Hispanic.

TABLE 3
Demographic Characteristics - ITT Population

| | Total Subjects |
|---|---|
| | N = 28 |
| Age (years) | |
| n | 28 |
| Mean (SD) | 24.9 (3.9) |
| Median | 24 |
| Range | 18-34 |
| Gender - n (%) | |
| Males | 19 (68) |
| Females | 9 (32) |
| Race - n (%) | |
| White | 28 (100) |
| Black/African American | 0 |
| Asian | 0 |
| Other | 0 |
| Ethnicity - n (%) | |
| Hispanic or Latino | 0 |
| Not Hispanic or Latino | 28 (100) |
| Height (in) | |
| n | 28 |
| Mean (SD) | 70.1 (4.1) |
| Median | 70.0 |
| Range | 63-79 |
| Weight (lb) | |
| n | 28 |
| Mean (SD) | 177.9 (34.6) |
| Median | 178.0 |
| Range | 112-250 |

At any time during Screening but at least prior to Day 1 of the first treatment period, subjects had their lower esophageal sphincter (LES) located to determine accurate placement of the pH probe.

Subjects were randomized on Day 1 of the first treatment period into 1 of 4 dosing sequences to receive a 9-day course of each one of the following daily treatment regimens in a crossover fashion:
Treatment A: 1 tablet PN 400/E30 (EC naproxen 500 mg and non-EC esomeprazole 30 mg) BID.
Treatment B: 1 tablet PN 400/E20 (EC naproxen 500 mg and non-EC esomeprazole 20 mg) BID.
Treatment C: 1 tablet PN 400/E10 (EC naproxen 500 mg and non-EC esomeprazole 10 mg) BID.
Treatment D: EC E20+naproxen (1 tablet non-EC naproxen 500 mg and 1 capsule EC esomeprazole 20 mg in the AM and 1 tablet non-EC naproxen 500 mg in the PM)

All treatments were administered by study personnel 60 minutes prior to meals (i.e. doses were taken 60 minutes prior to breakfast (after an over night fast, for the AM dose) and/or 60 minutes prior to dinner (for the PM dose) for 9 days). The study medications administered BID were administered approximately 10 hours apart. Prior to administration of the Day 1 AM dose of study drug, the pH probe was placed to monitor intragastric pH for a period of 24 hours. The distal electrode was placed 10 cm below the LES with the proximal electrode placed 5 cm above the LES using the LES locator and/or by use of formal esophageal manometry by the investigator. The position of the distance of the electrode from the nostrils was recorded to facilitate the 24-hour intragastric pH assessments.

In addition, a pre-AM dose blood sample was collected. Post-AM dose blood samples were drawn approximately: 10, 20, 30 and 45 minutes and 1, 1.5, 2, 2.5, 3, 4, 6, 8, 10 (pre-PM dose blood sample), 10.17, 10.33, 10.5, 10.75, 11, 11.5, 12, 12.5, 13, 14, 16, 18, and 24 hours for PK assessments. After AM dosing on Day 2, subjects were discharged from the Phase 1 unit and instructed to return for the next dosing in the PM of Day 2 and on Days 3-8 to receive the AM and PM doses. Subjects were confined to the Phase 1 unit in the PM of Day 8 in preparation for the 24-hour PK and pH assessments on Day 9. Monitoring of pH and blood sample collection on Day 9 was as described above for Day 1.

The pH probe was removed in the AM on Day 10. Final PK samples were collected in the AM of Day 10.

Subjects were instructed to return to the Phase 1 unit for subsequent treatment periods and reminded of the timing for the next treatment period (i.e., the wash-out period of at least 12 days between treatments). In each subsequent treatment period, the same procedures were performed as during the first period, and final study procedures were performed on Day 10 of the last treatment period or whenever a subject discontinued from the study.

The PK measurements used in this study are assessments commonly used in Phase I studies.

### Measuring Intragatric pH

The procedure used to measuring intragastric pH is standard commonly used in assessing the pharmacodynamic effect of acid-suppressing drugs. A Medtronics Digitrapper pH data logger (Medtronics, Minneapolis, Minn.) was used to record pH. The 24-hour pH assessments were performed on Days 1 and 9. The pH recording system measured the difference in potential between the recording and reference electrodes in the tip of the probe and stored this value every couple of seconds. The pH data was provided to a third party blinded to the assigned treatment groups. The third party evaluated the data to determine the validity of the pH recordings based on the following established criteria: At least 20 hours of valid pH data within a pre-specified reference range; No technical failures of the pH recording; and Less than 1 continuous hour with pH data outside the reference range.

### Collection of Samples

PK blood samples were collected in 6 mL sodium heparin VACUTAINER tubes and stored on ice until centrifuged within 30 minutes of sample draw for approximately 10 minutes at 3000 rpm (approximately 1800×g) in a refrigerated centrifuge maintained at approximately 4° C. Plasma supernatant was withdrawn and frozen at −20° C. or colder within 60 minutes of collection. Samples were divided into 2 equal aliquots, with 1 tube being used for naproxen analysis and the other for esomeprazole analysis. Samples were kept frozen at ±20° C. and shipped overnight to PPD Development, Richmond Va. for analysis at the end of the study.

### Analysis of Esomeprazole in Plasma

### Assay Methods

Esomeprazole is optically stable and the degree of conversion from the S to the R enantiomer in humans is negligible. Thus sample analysis for esomeprazole was performed using the validated high performance liquid chromatography tandem mass spectrometry (HPLC/MS/MS) method for omeprazole. This method was developed and validated by PPD Development, Richmond, Va.

Assay methods were generally performed as essentially described hereinbelow:

A 100 μL aliquot of human plasma containing the anti-coagulant (sodium heparin) and the analytes was fortified with the internal standard (deuterated analog of omeprazole). The analytes were isolated by solid phase extraction (SPE) using a Phenomenex Strata-X (10 mg) 96-well SPE plate. The final extract was analyzed by HPLC with MS/MS detection using a Micromass Quattro Micro, triple quadrupole instrument. Chromatographic retention and separation of the analytes were obtained on Betasil Silica-100 analytical column (3×50 mm, 5 μm particle size) using a gradient mobile phase program. Mobile phase A consisted of 0.1% formic acid in acetonitrile and mobile phase B consisted of 0.1% formic acid. The analytes were detected by MS/MS with positive electrospray ionization in the mode of multiple reaction monitoring (MRM), with ions monitored for omeprazole (m/z 346→198) and deuterated omeprazole (m/z 349→198).

Quantification was by analyte to internal standard peak area ratio. The linear range of quantitation was 1 to 1000 ng/mL in human plasma, with a lower limit of quantification (LLOQ) of 1 ng/mL. The assay was validated in terms of specificity, precision, accuracy, and sample stability.

### Assay Performance

A set of 8 calibration standards ranging from 1.00 to 1000 ng/mL and quality control (QC) samples at 5 different concentrations (2.60, 8.00, 30.0, 130, and 750 ng/mL) of each analyte were prepared and stored at −20° C. Between-batch precision and accuracy for analysis of the QC samples were determined from batch analyses of clinical samples in this study. Precision was measured as the percent coefficient of variation (% CV) of the set of values obtained for each QC level. Accuracy was expressed as the percent difference of the mean value from the theoretical concentration at each QC level.

The inter-assay CV of the QCs for the omeprazole runs ranged from 3.44% to 5.88%, with mean percent differences from theoretical ranging from 0.329% to 1.80%. The differences of back-calculated calibration curve values from nominal values ranged from −2.87% to 1.54%. For the analytical runs, which contained diluted subject samples, the appropriate level quality control pool was diluted and analyzed in a similar manner to validate the dilution of study samples. The % CV of the diluted QCs for the run ranged from 0.599% to 2.74% with mean percent differences from theoretical ranging from −1.24% to 3.99%.

### Analysis of Naproxen in Plasma

### Assay Methods

Concentrations of naproxen in human plasma were generally determined essentially as described hereinbelow using a validated HPLC method with fluorescence detection developed and validated at PPD Development, Richmond, Va.

A 100 μL aliquot of human plasma containing sodium heparin and the analytes was combined with the internal standard solution (2-naphthylacetic acid) and diluted with potassium chloride. The analyte and the internal standard were isolated using liquid-liquid extraction. Chromatographic retention and separation of the analytes was obtained on a Symmetry C18 column (4.6×150 mm, 5 μm particle size) using an isocratic mobile phase consisting of 45% acetonitrile: 55% 14.8 mM phosphate buffer. The analytes were detected by fluorescence using excitation and emission wavelengths of 230 nm and 370 nm, respectively.

Quantification was by analyte to internal standard peak height ratio. The nominal range of the method was 0.10 to 100 μg/mL for naproxen in human plasma with an LLOQ of 0.10 μg/mL.

### Assay Performance

A set of 8 calibration standards ranging from 0.1 to 100 μg/mL and QC samples at 5 different concentrations (0.28, 0.80, 3.0, 12.0 and 76.0 μg/mL) of the analyte were prepared and stored at −20° C. Between-batch precision and accuracy for analysis of the QC samples were determined from batch analyses of clinical samples in this study. Precision was measured as the % CV of the set of values obtained for each QC level. Accuracy was expressed as the percent difference of the mean value from the theoretical concentration at each QC level.

The inter-assay CV of the QCs for the naproxen runs ranged from 2.22% to 9.59%, with mean percent differences from theoretical ranging from −3.59% to 0.933%. The differences of back-calculated calibration curve values from nominal values ranged from −4.73% to 3.79%. For the analytical run that contained diluted subject samples, the appropriate level of QC samples was diluted and analyzed in a similar manner to validate the dilution of study samples. The % CV of the diluted QCs for the run was 4.12% with a mean percent difference from theoretical of −1.54%.

### Statistics and Analysis

Data were summarized by reporting the frequency and percentage of subjects in each category for categorical and ordinal measures, and means, standard deviation or standard error, medians, and ranges for continuous measures. All statistical analyses and data listings were completed using the SAS® system, version 8.2 or higher.

Three analysis populations were used for analysis:

1) Intent-to-Treat (ITT) population: all randomized subjects who had valid pH data for at least 1 treatment period. A subject was considered to have valid pH data for each treatment period if the subject received all doses of study medication per protocol, had at least 20 hours of valid pH data determined by the clinical investigator, did not have technical failures of the pH recording, and did not have one continuous hour or more with pH data outside the reference range.

2) Per-Protocol (PP) population: all ITT subjects who had valid pH data for all four treatment periods and did not violate the protocol in a way that would have significantly impacted the evaluation of PD endpoints.

3) PK population: all randomized subjects who received all doses of study medication for at least one treatment period and had adequate blood sampling to determine the PK parameters of the study drugs.

### Pharmacodynamic Endpoints

For each Digitrapper session, separate plots of the esophageal and intragastric pH readings for each subject were prepared and reviewed for non-valid pH data. Particular attention was paid to recorded pH values that were outside the range from 0.6 to 8.0. Values considered by the investigator to be unlikely to have occurred due to a reasonable expectation of method variability, and which persisted to such an extent as to suggest possible transient unreliability of the equipment, were identified and excised from the database. Other apparent ‘flat-lining’ of the pH readings could also result in a determination of non-valid data.

The PD endpoints were summarized by treatment and analyzed by Analysis of Variance (ANOVA). The ANOVA model included sequence, period, and treatment as fixed effects, and subject within sequence as a random effect. The least square (LS) means for each treatment, the difference of LS means between each of the PN 400 treatments and the active control, and 95% confidence intervals (CIs) for all treatment differences were calculated. Both ITT and PP populations were used for the PD analysis. The PP population was the primary analysis population. In addition, the percent time of pH>3.0 and ≧5.0 on Days 1 and 9 was analyzed in a similar fashion as the percent time of pH>4.0. Mean pH data over 24 hours on Days 1 and 9 were plotted by treatment.

From a previous study, the within-subject standard deviation (SD) of percent time of pH>4.0 was 10%. The current study planned to enroll 28 subjects with the aim to have 24 evaluable subjects for analysis. A total of 24 subjects provides 80% power to reject the null hypothesis that the difference between each of the PN 400 treatments and the active control in percent time of pH>4.0 over 24 hours is ≧−8% using a pairwise t-test with a one-sided significance level of 0.05.

Primary Pharmacodynamic Endpoint: Percent Time Intragastric pH>4.0 on Day 9

Results from Day 9 are set forth in Table 4. On Day 9, both PN 400/E30 and PN 400/E20 treatments resulted in a greater percent time with intragastric pH>4.0 than treatment with EC E20+naproxen. PN 400/E10 had the lowest percent time with intragastric pH>4.0 and was also the most variable treatment as evidenced by the high % CV in Table 4.

TABLE 4
Percent Time of pH Greater
than 4.0 - Day 9 - Per-Protocol Population

| | | | | D |
|---|---|---|---|---|
| | A | B | C | EC E20 + |
| | PN 400/E30 | PN 400/E20 | PN 400/E10 | naproxen |
| | N = 25 | N = 25 | N = 25 | N = 25 |
| % Time of pH >4.0 | | | | |
| Mean (SD) | 76.50 (12.26) | 71.35 (13.01) | 40.85 (22.51) | 56.85 (10.06) |
| Median | 78.79 | 70.42 | 35.76 | 55.14 |
| % Coefficient of variation | 16 | 18 | 55 | 18 |
| Range | 49.79-95.32 | 51.76-97.61 | 10.30-85.26 | 40.63-75.51 |
| LS Mean (SD) | 76.75 (3.02) | 71.46 (3.02) | 41.09 (3.02) | 57.23 (3.02) |
| | A vs. D | B vs. D | C vs. D | |
| LS Mean Difference (SE) | 19.52 (3.25) | 14.23 (3.25) | −16.14 (3.25) | — |
| 95% Confidence Interval | 13.04-26.01 | 7.75-20.71 | −22.26-−9.66 | — |

On Day 1, the LS mean percent time intragastric pH>4.0 ranged from 13% with PN 400/E10 to 28% with PN 400/E30. Treatment differences compared to EC E20+naproxen were small. Only PN 400/E30 (28%) had a statistically significant, greater percent time with pH>4.0 compared to EC E20+naproxen (21%).

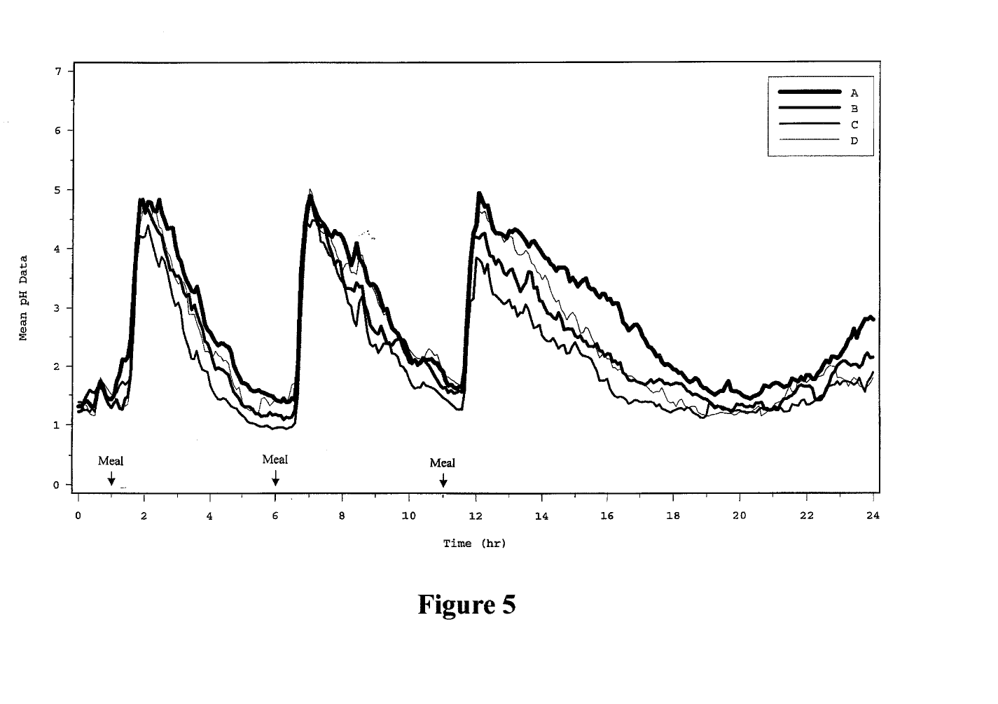

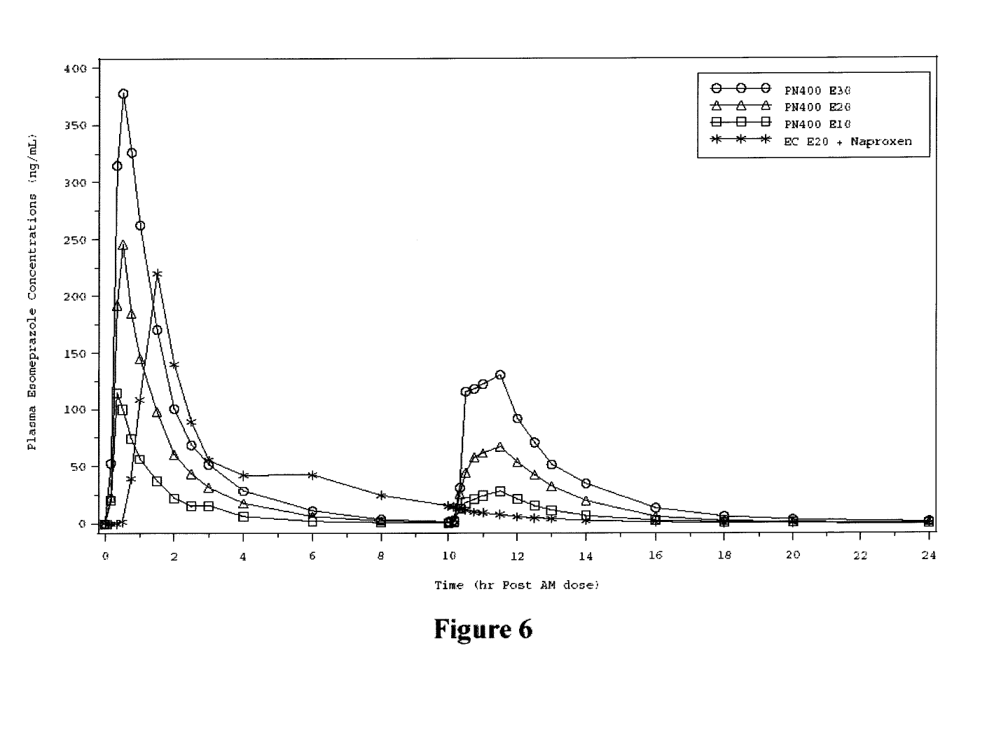

As shown in FIG. 4, there were three increases in pH throughout the day which were associated with food intake at 1, 6 and 11 hours, for breakfast, lunch and dinner, respectively. These increases in pH occurred approximately one hour after each meal for all treatments. Following the AM dose of PN 400 on Day 9, the pH increase occurred approximately one hour earlier than the food induced increase in pH (see also FIG. 5). After the AM dose of EC E20+naproxen, the pH increase occurred at least 30 minutes later than PN 400.

The overall pH profiles on Day 9 showed an esomeprazole dose-related effect on intragastric pH beyond the influence of food intake. The effect on intragastric pH profiles was similar between PN 400/E30 and PN 400/E20, with each of these treatments reflecting a slower return of gastric contents to lower pH levels after food intake than either the PN400/E10 or EC E20+naproxen treatments.

Results on Day 9 for the ITT population were similar to those from the PP population. The initial Day 9 pH measurements from all treatments showed the mean intragastric pH after an overnight fast was between 2.0 and 3.0, which was higher than the initial pH (between 1.0 and 2.0) on Day 1 (FIG. 5).

As shown in the table above, the primary PD response of this study, i.e., percent time intragastric pH>4.0 on Day 9, increased with esomeprazole dose in the PN 400 treatments. However, a greater increase in the primary PD response was observed when esomeprazole dose increased from 10 to 20 mg in PN 400, i.e., from 40.9 to 76.5%. There was only a small increase in intragastric pH>4.0, from 71.4 to 76.5%, as the esomeprazole dose increased from 20 to 30 mg in PN 400.

Secondary Pharmacodynamic Endpoint: Percent Time Intragastric pH>4.0 on Day 1

Results from Day 1 are set forth in Table 5. The LS mean percent time intragastric pH>4.0 ranged from approximately 13% with PN 400/E10 to approximately 28% with PN 400/E30.

TABLE 5
Percent of Time with Intragastric pH
Greater than 4.0 - Day 1- Per-Protocol Population

| | Treatment | | | |
|---|---|---|---|---|
| | | | | D |
| | A | B | C | EC E20 + |
| | PN 400/E30 | PN 400/E20 | PN 400/E10 | Naproxen |
| | N = 25 | N = 25 | N = 24 | N = 25 |
| % Time pH >4.0 | | | | |
| Mean (SD) | 27.79 (22.63) | 20.50 (16.61) | 12.81 (11.11) | 21.34 (13.63) |
| Median | 19.96 | 15.26 | 9.09 | 16.82 |
| % CV | 81 | 81 | 87 | 64 |
| Range | 1.77-89.61 | 4.35-74.40 | 3.00-53.75 | 3.16-58.20 |
| LS Mean (SE) | 27.90 (3.31) | 20.58 (3.31) | 12.66 (3.35) | 21.51 (3.31) |
| | A vs. D | B vs. D | C vs. D | |
| LS Mean Difference (SE) | 6.39 (3.18) | −0.92 (3.18) | −8.85 (3.22) | — |
| 95% Confidence Interval | 0.04-12.75 | −7.28-5.43 | −15.28-−2.42 | — |

Mean pH data over 24 hours on Day 1 is presented in FIG. 5. The pH profiles on Day 1 showed that following an overnight fast, the mean baseline intragastric pH was low, between 1.0 and 2.0, prior to any treatment. There were three pH increases above pH 4.0 throughout the day that were associated with food intake at 1, 6 and 11 hours. The increase in intragastric pH occurred at approximately one hour after each meal for all treatments. There was only a minimal effect of any of the study treatments on intragastric pH, beyond the effect of food, throughout the first 24 hours on the first day of treatment.

### Other Pharmacodynamic Findings

Analysis of percent time of pH>3.0 and >5.0 on Day 9 resulted in a similar pattern statistically as that of the primary endpoint of percent time pH>4 on Day 9 for PP population, with PN 400/E30 and PN 400/E20 showing a greater acid-reducing capacity than EC E20+naproxen, which had a greater capacity than PN 400/E10, based on LS mean differences and 95% CIs. Analysis results of percent time of pH>3.0 and >5.0 on Day 1 were similar as results for the primary endpoint of % time pH>4 on Day 1 based on the PP population. ITT population results were similar to those of the PP population.

As PN 400 is dosed BID, the individual time intervals corresponding to this dosing regimen, i.e., 0-10 hours and 10-24 hours, were analyzed for percent time intragastric pH>4.0 on Day 9. The results indicated that for the 0-10 hours period, PN 400/E30 treatment resulted in a greater percent time with intragastric pH>4.0 (84%) than treatment with EC E20+naproxen (71%). While PN 400/E20 also had a high percent time intragastric pH>4.0 (79%), the results were not statistically significantly different from the EC E20+naproxen treatment. With BID dosing, both PN400/E30 and PN400/E20 had greater percent time intragastric pH>4.0 (71% and 66%, respectively) compared to treatment with EC E20+naproxen (47%) for the 10-24 hour treatment interval. The PN400/E10 treatment had a lower percent time intragastric pH>4.0 compared to treatment with EC E20+naproxen for both the 0-10 hours (52%) and the 10-24 hours (33%) treatment interval.

### Pharmacokinetic Endpoints

On Day 1, PK data analysis was performed for esomeprazole and naproxen plasma profiles obtained from 28 subjects completing PN 400/E30, PN 400/E20 and EC E20+naproxen; and 27 subjects completing PN 400/E10.

On Day 9, PK data analysis was performed for esomeprazole or naproxen plasma profiles from 28 subjects completing PN 400/E30 and EC E20+naproxen, and 27 subjects completing PN 400/E20 and PN 400/E10.

PK parameters for esomeprazole were determined following the three different PN 400 treatments and PK parameters for naproxen were determined following each of the 4 treatments including peak plasma concentration (Cₘₐₓ) on Days 1 and 9; time to peak plasma concentration (tₘₐₓ) on Days 1 and 9; area under the plasma concentration vs time curve from time zero to the last time point with measurable drug concentration (AUC₀₋ₜ) on Days 1 and 9; and the terminal half-life (t1/2), if possible, following both the AM and PM doses on Days 1 and 9. In addition, the AUC from time zero (time of dosing) to 10 hours post-AM dose (AUC_{0-10,am}) and AUC from time zero (time of dosing) to 14 hours post-PM dose (AUC_{0-14,pm}) and a total daily AUC (AUC₀₋₂₄) were determined on Days 1 and 9. PK parameters for esomeprazole following EC E20+naproxen included Cₘₐₓ, tₘₐₓ, AUC₀₋ₜ, t1/2, and AUC₀₋₂₄ following the AM dose on both Days 1 and 9.

Statistical analysis was performed using Analysis of Variance (ANOVA) to determine the point estimate and 90% CI of the Day 9 to Day 1 ratios for the following parameters for both naproxen and esomeprazole C_{max,am}, C_{max,pm}, AUC_{0-10,am}, AUC_{0-14,pm}, and AUC₀₋₂₄.

Plasma esomeprazole and naproxen concentration vs. time data are listed by treatment, study day and subject. The concentration data were summarized by treatment and study day at each nominal (or scheduled) sampling time using descriptive statistics including mean, SD, % CV, median, minimum and maximum. Plasma concentrations below the LLOQ (i.e., 1 ng/mL for esomeprazole, and 0.10 μg/mL for naproxen) were treated as a zero value for calculating descriptive statistics. The mean/median value at a time point with one or more below the LLOQ (BQL) values was reported unless the mean/median value was below the LLOQ of the assay, in which case the value was reported as BQL. Individual subject plasma concentration vs time curves were plotted against the actual sampling time, and the mean/median plasma concentration vs. time curves were plotted against nominal sampling time by treatment.

The plasma concentration vs. time data of each analyte were subjected to non-compartmental analysis using WinNonlin version 4.1 (Pharsight Corporation, Mountain View, Calif.). The actual blood sampling time for each sample was used for PK data analysis. For analyte concentrations resulting from BID doses, i.e., esomeprazole in Treatments A, B, and C or naproxen in all 4 treatments, PK analysis was performed separately for the plasma profiles obtained after the AM and PM doses. Thus, the actual post-dose sampling times for concentration vs. time profiles after the PM dose were calculated based on the elapsed time from the actual dosing time of the PM dose.

For PK analysis, plasma concentrations below the LLOQ (BQL value) in individual profiles of each analyte were handled as follows. If the value occurred in a profile during the absorptive phase of the profile, i.e., before the maximum concentration in a profile was observed, it was assigned a value of zero concentration. A single BQL value occurring between two measurable analyte concentrations, not in the absorptive phase of a profile, was generally omitted. If two or more BQL values occurred in succession, post peak time (or during the elimination phase), the profile was determined to have terminated at the last time point with measurable analyte concentration of the profile.

PK parameters calculated for esomeprazole (following each of the three PN 400 treatments) and for naproxen (following each of the 4 treatments) on Days 1 and 9 included the following: Maximum observed plasma concentration following the AM dose (C_{max,am}) and following the PM dose (C_{max,pm}); Time to peak plasma concentration following the AM dose (t_{max,am}) and following the PM dose (t_{max,pm}); Time to the first measurable plasma concentration following the AM dose (tlag,am) and following the PM dose (t_{lag,pm}); Area under the plasma concentration vs. time curve (AUC) from time zero to the last time point with measurable drug concentration (tₗₐₛₜ) following the AM dose (AUC_{0-t,am}) and following the PM dose (AUC_{0-t,pm}), calculated using the linear-up and log-down trapezoidal method in WinNonlin; Apparent first-order elimination rate constant (λ_{z,am} and λ_{z,pm}), determined, if data permit, by the slope of the apparent terminal log-linear phase of the plasma drug concentration vs. time curve using at least 3 time points; Apparent plasma half-life (t1/2), if data permit, determined as 0.693/λ_{z} following the AM and PM doses, i.e., t1/2,ₐₘ and t_{1/2,pm}, respectively; AUC from time zero (time of AM dosing) to 10 hours after the AM dose (AUC_{0-10,am}), if necessary, with extrapolation using λ_{z, am} estimate from tₗₐₛₜ to 10 hrs post the AM dose; AUC from time zero (time of PM dosing) to 14 hours after the PM dose (AUC_{0-14,pm}), if necessary, with extrapolation using λ_{z,pm} estimate from tₗₐₛₜ to 14 hrs post PM dose; and Total daily AUC (AUC₀₋₂₄) from time zero (time of AM dosing) to 24 hours after the AM dose, which is determined as AUC_{0-10,am}+AUC_{0-14,pm}.

The PK parameters calculated for esomeprazole following Treatment D after the AM dose on Days 1 and 9 included Cₘₐₓ, tₘₐₓ, AUC₀₋ₜ, t1/2 and AUC₀₋₂₄. The same methods as described above were applied.

Descriptive statistics, including mean, SD, % CV, median, minimum and maximum, were calculated for all PK parameters of naproxen and esomeprazole by treatment and study day. Geometric mean and associated 95% confidence interval (CI) were also calculated for all PK parameters, except tₘₐₓ.

Statistical analysis was performed using ANOVA to determine the point estimate and associated 90% CI of the Day 9 to Day 1 ratios for the following parameters: C_{max,am}, C_{max,pm}, AUC_{0-10,am}, AUC_{0-14,pm}, and AUC₀₋₂₄ for esomeprazole data in each of the three PN 400 treatments, and for naproxen data in each of the 4 treatments. For esomeprazole data from Treatment D, the Day 9 to Day 1 ratios for Cₘₐₓ and AUC₀₋₂₄ were determined. Natural log-transformed Cₘₐₓ and AUC values were used for the analyses, thus geometric least-squares mean ratios for each parameter were determined.

Dose proportionality in the C_{max,am}, C_{max,pm}, AUC_{0-10,am}, AUC_{0-14,pm}, and AUC₀₋₂₄ of esomeprazole from the three PN400 treatments was analyzed on Days 1 and 9 separately, using the power model as follows:

y=a*(dose)^{b}

ln(y)=ln(a)+b*ln(dose)

where y is the PK parameter value and ln=natural log. The power model included ln(dose) and period as fixed effects and subject as a random effect.

### Esomeprazole

Table 6 summarizes the results from the PK analysis that was performed for esomeprazole plasma concentration vs. time data for the 28 subjects who completed PN 400/E30 and EC E20+naproxen treatments and 27 subjects who completed PN 400/E10 treatment on Days 1 and 9; and 28, and 27 subjects who completed PN 400/E20 treatment on Day 1 and Day 9, respectively.

TABLE 6
Summary of Esomeprazole Pharmacokinetic Results

| | | | | AUC_{0-10, am} or | | |
|---|---|---|---|---|---|---|
| | Day/Dose | Cₘₐₓ | tₘₐₓ | AUC_{0-14, pm} | AUC₀₋₂₄ | t½ |
| Treatment | Time | (ng/mL) | (hr) | (hr * ng/mL) | (hr * ng/mL) | (hr) |
| A | 1 | 487 | 0.50 | 591 | | 0.892 |
| PN 400/E30 | AM | (82) | (0.33-1.50) | (108) | | (35) |
| | 1 | 187 | 1.50 | 388 | 978 | 1.11 |
| | PM | (132) | (0.33-4.00) | (137) | (115) | (62) |
| | 9 | 1584 | 0.50 | 2779 | | 1.26 |
| | AM | (39) | (0.17-1.50) | (45) | | (25) |
| | 9 | 810 | 1.00 | 2066 | 4911 | 1.46 |
| | PM | (59) | (0.33-8.00) | (53) | (42) | (34) |
| B | 1 | 292 | 0.50 | 350 | | 0.846 |
| PN 400/E20 | AM | (77) | (0.20-1.50) | (113) | | (42) |
| | 1 | 96.6 | 1.49 | 206 | 556 | 0.994 |
| | PM | (104) | (0.33-3.00) | (141) | (119) | (55) |
| | 9 | 715 | 0.50 | 1216 | | 1.12 |
| | AM | (52) | (0.17-1.50) | (69) | | (33) |
| | 9 | 428 | 0.75 | 919 | 2134 | 1.31 |
| | PM | (73) | (0.33-3.00) | (84) | (74) | (42) |
| C | 1 | 138 | 0.33 | 148 | | 0.810 |
| PN 400/E10 | AM | (71) | (0.17-3.10) | (111) | | (48) |
| | 1 | 35.3 | 1.50 | 85.7 | 237 | 0.878 |
| | PM | (84) | (0.33-3.00) | (179) | (133) | (50) |
| | 9 | 278 | 0.33 | 368 | | 0.860 |
| | AM | (57) | (0.17-1.00) | (89) | | (41) |
| | 9 | 97.6 | 1.00 | 223 | 602 | 1.09 |
| | PM | (136) | (0.33-2.00) | (134) | (103) | (47) |
| D | 1 | 282 | 1.50 | 520 | 580 | 1.09 |
| EC E20 + | AM | (66) | (1.00-16.0) | (64) | (67) | (44) |
| naproxen | 9 | 435 | 1.50 | 1046 | 1212 | 1.27 |
| | AM | (48) | (1.00-14.0) | (54) | (47) | (36) |

Values are mean (% CV) for all parameters, except for tmax, which are median (range).

Following oral administration of PN 400 on Day 1, esomeprazole concentrations were measurable at 10 minutes after the AM dose, and at 20-30 minutes after the PM dose. Plasma esomeprazole concentrations after the PM dose were lower than those after the AM dose on both days. Cₘₐₓ and AUCs of esomeprazole increased nearly dose proportionally after the AM dose on Day 1, but more than dose proportionally after the PM dose on Day 1 and both the AM and PM doses on Day 9. Esomeprazole concentrations were much higher on Day 9 than on Day 1 for each PN 400 treatment. The geometric least-squares mean AUC₀₋₂₄ ratios, Day 9 to Day 1, were 7.13, 4.10, and 2.26 for treatment with PN 400/E30, PN 400/E20, and PN 400/E10, respectively.

Following EC E20+naproxen treatment, the first measurable esomeprazole concentration was at 0.5 to 1.5 hrs post dose. To evaluate the effect of esomeprazole in the PN400/E20 treatment group with the EC E20 in the EC E20+naproxen treatment group, the PK parameters from PN400/E20 and EC E20+naproxen treatment groups were compared. On Day 1, esomeprazole C_{max,am} mean values were approximately equal for the PN400/E20 and EC E20+naproxen treatments (292 and 282 ng/mL, respectively). The AUC₀₋₁₀ mean values on Day 1 from the PN400/E20 treatment were approximately two-thirds that of the EC E20+naproxen treatment (350 vs. 520 hr*ng/mL, respectively). By Day 9, however, the esomeprazole AUC₀₋₁₀ for the PN400/E20 treatment group was greater than the EC E20+naproxen treatment group (1216 vs 1046 hr·ng/mL, respectively) and C_{max,am} from the PN400/E20 treatment group was almost double that of the EC E2030 naproxen treatment group (715 vs 435 ng/mL, respectively).

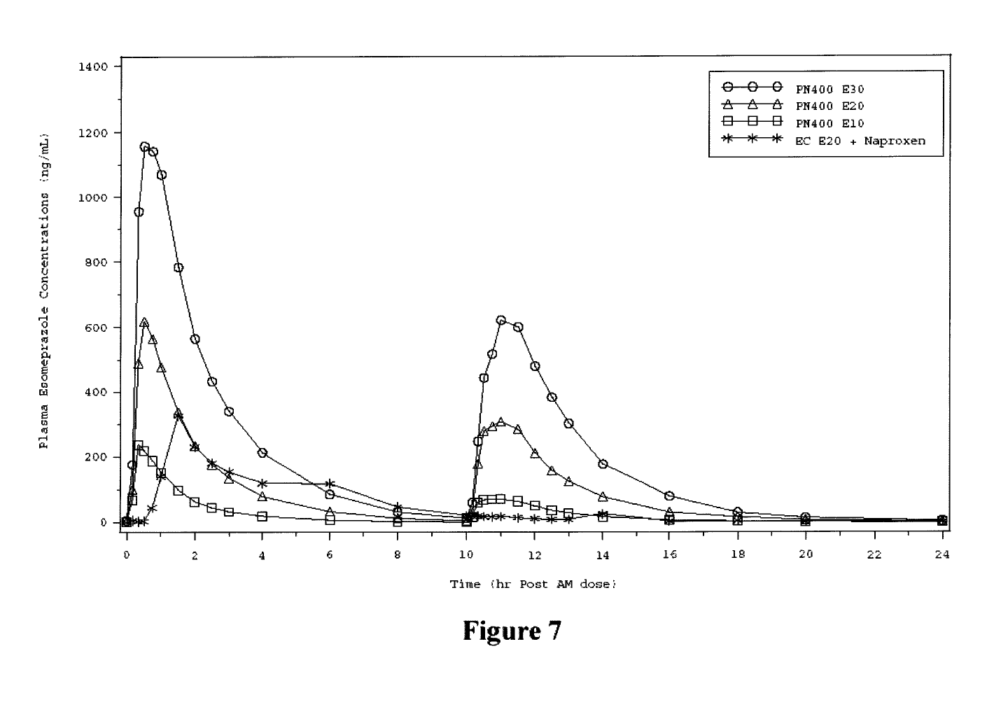

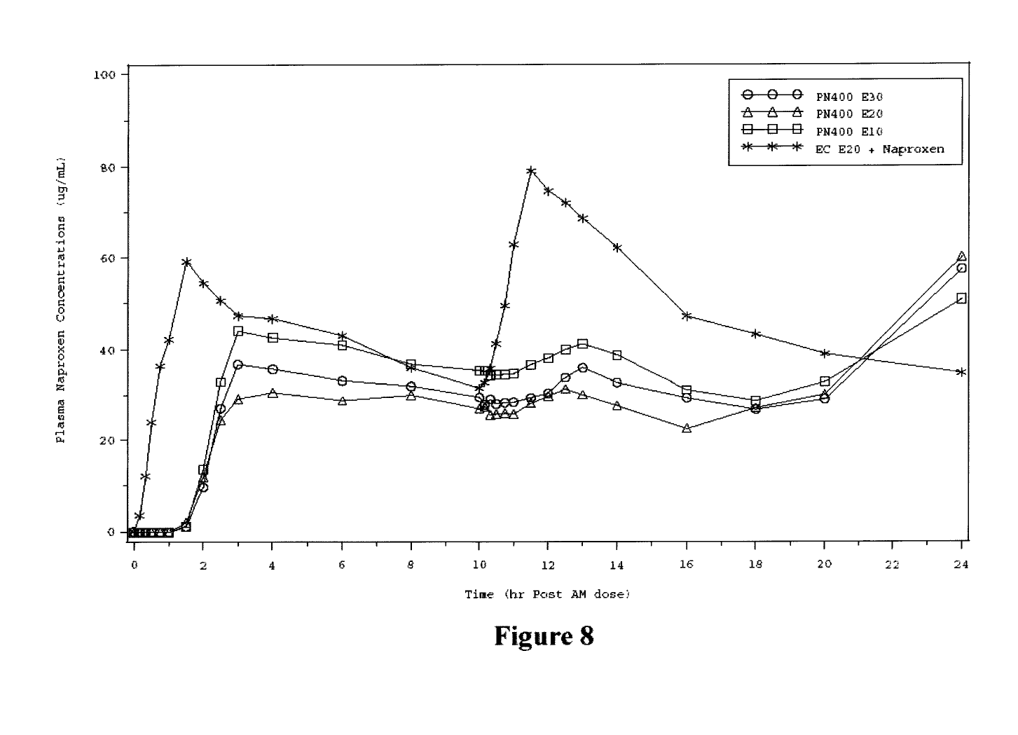

Mean and median plasma esomeprazole concentration vs time profiles following all four treatments were plotted and the mean plots are presented in FIGS. 6 and 7. These figures demonstrate that esomeprazole concentrations following AM or PM doses on Day 1 or Day 9 increased with the esomeprazole dose in the PN 400 treatments. Furthermore, on both Days 1 and 9, esomeprazole concentrations after the AM dose were higher than those after the PM dose for each PN 400 treatment. Following EC E20+naproxen treatment, the mean profiles demonstrate a delayed absorption peak on both Days 1 and 9 as compared to PN 400. In addition, peak esomeprazole concentrations following EC E20+naproxen treatment were lower than those following PN 400/E20, especially on Day 9 (about 50% lower).

The pharmacokinetic parameters of esomeprazole following the AM and PM doses on Day 1 and Day 9 of each treatment group are summarized in the tables below.

TABLE 7
Summary of Esomeprazole Pharmacokinetic Parameters by Study
Day and Dose Time for Treatment A (PN 400/E30)

| | | | | | | AUC_{0-10, am} or | | |
|---|---|---|---|---|---|---|---|---|
| | Dose | | Cₘₐₓ | tₘₐₓ | AUC₀₋ₜ | AUC_{0-14, pm} | AUC₀₋₂₄ | t½ |
| Day | Time | Statistics | (ng/mL) | (hr) | (hr * ng/mL) | (hr * ng/mL) | (hr * ng/mL) | (hr) |
| 1 | AM | Mean | 487 | | 588 | 591 | | 0.892 |
| | n = 28 | % CV | 82 | | 109 | 108 | | 35 |
| | | Median | 325 | 0.50 | 352 | 354 | | 0.810 |
| | | Min | 52.1 | 0.33 | 59.4 | 61.0 | | 0.520 |
| | | Max | 1400 | 1.50 | 3087 | 3087 | | 1.96 |
| 1 | PM | Mean | 187 | | 385 | 388 | 978 | 1.11 |
| | n = 28 | % CV | 132 | | 138 | 137 | 115 | 62 |
| | | Median | 114 | 1.50 | 204 | 207 | 579 | 0.861 |
| | | Min | 21.1 | 0.33 | 37.4 | 40.3 | 101 | 0.593 |
| | | Max | 1290 | 4.00 | 2315 | 2315 | 5402 | 3.89 |
| 9 | AM | Mean | 1584 | | 2778 | 2779 | | 1.26 |
| | n = 28 | % CV | 39 | | 46 | 45 | | 25 |
| | | Median | 1560 | 0.50 | 2586 | 2586 | | 1.18 |
| | | Min | 384 | 0.17 | 874 | 879 | | 0.83 |
| | | Max | 3520 | 1.50 | 5841 | 5841 | | 1.80 |
| 9 | PM | Mean | 810 | | 1990 | 2066 | 4911 | 1.46^{a} |
| | n = 28 | % CV | 59 | | 57 | 53 | 42 | 34 |
| | | Median | 749 | 1.00 | 2090 | 2159 | 5488 | 1.38 |
| | | Min | 13.7 | 0.33 | 21.7 | 342 | 1519 | 0.75 |
| | | Max | 1970 | 8.00 | 4956 | 4956 | 9770 | 2.91 |

^{a}n = 27

TABLE 8
Summary of Esomeprazole Pharmacokinetic Parameters by
Study Day and Dose Time for Treatment B (PN 400/E20)

| | | | | | | AUC_{0-10, am} or | | |
|---|---|---|---|---|---|---|---|---|
| | Dose | Statis- | Cₘₐₓ | tₘₐₓ | AUC₀₋ₜ | AUC_{0-14, pm} | AUC₀₋₂₄ | t½ |
| Day | Time | Tics | (ng/mL) | (hr) | (hr * ng/mL) | (hr * ng/mL) | (hr * ng/mL) | (hr) |
| 1 | AM | Mean | 292 | | 348 | 350 | | 0.846 |
| | n = 28 | % CV | 77 | | 114 | 113 | | 42 |
| | | Median | 209 | 0.50 | 245 | 248 | | 0.696 |
| | | Min | 47.1 | 0.20 | 57.0 | 58.1 | | 0.434 |
| | | Max | 916 | 1.50 | 1971 | 1971 | | 1.90 |
| 1 | PM | Mean | 96.6 | | 203 | 206 | 556 | 0.994 |
| | n = 28 | % CV | 104 | | 144 | 141 | 119 | 55 |
| | | Median | 71.7 | 1.49 | 106 | 108 | 362 | 0.844 |
| | | Min | 16.0 | 0.33 | 24.3 | 28.6 | 86.7 | 0.410 |
| | | Max | 439 | 3.00 | 1459 | 1459 | 3429 | 2.82 |
| 9 | AM | Mean | 715 | | 1215 | 1216 | | 1.12 |
| | n = 27 | % CV | 52 | | 70 | 69 | | 33 |
| | | Median | 700 | 0.50 | 947 | 948 | | 1.03 |
| | | Min | 112 | 0.17 | 186 | 188 | | 0.485 |
| | | Max | 1300 | 1.50 | 2931 | 2931 | | 1.82 |
| 9 | PM | Mean | 428 | | 914 | 919 | 2134 | 1.31 |
| | n = 27 | % CV | 73 | | 85 | 84 | 74 | 42 |
| | | Median | 373 | 0.75 | 603 | 642 | 1727 | 1.32 |
| | | Min | 30.5 | 0.33 | 59.3 | 63.5 | 288 | 0.686 |
| | | Max | 1300 | 3.00 | 2931 | 2931 | 5737 | 3.10 |

TABLE 9
Summary of Esomeprazole Pharmacokinetic Parameters by
Study Day and Dose Time for Treatment C (PN 400/E10)

| | | | | | | AUC_{0-10, am} or | | |
|---|---|---|---|---|---|---|---|---|
| | Dose | Statis- | Cₘₐₓ | tₘₐₓ | AUC₀₋ₜ | AUC_{0-14, pm} | AUC₀₋₂₄ | t½ |
| Day | Time | Tics | (ng/mL) | (hr) | (hr * ng/mL) | (hr * ng/mL) | (hr * ng/mL) | (hr) |
| 1 | AM | Mean | 138 | | 143 | 148 | | 0.810 |
| | n = 27 | % CV | 71 | | 115 | 111 | | 48 |
| | | Median | 123 | 0.33 | 84.8 | 105 | | 0.703 |
| | | Min | 24.4 | 0.17 | 35.0 | 36.1 | | 0.454 |
| | | Max | 370 | 3.10 | 882 | 882 | | 2.21 |
| 1 | PM | Mean | 35.3 | | 80.6 | 85.7^{b} | 237^{b} | 0.878^{a} |
| | n = 27 | % CV | 84 | | 188 | 179 | 133 | 50 |
| | | Median | 32.1 | 1.50 | 41.8 | 48.7 | 155 | 0.734 |
| | | Min | 4.2 | 0.33 | 8.40 | 9.80 | 45.9 | 0.593 |
| | | Max | 141 | 3.00 | 818 | 818 | 1700 | 2.67 |
| 9 | AM | Mean | 278 | | 366 | 368 | | 0.860 |
| | n = 27 | % CV | 57 | | 90 | 89 | | 41 |
| | | Median | 242 | 0.33 | 223 | 224 | | 0.653 |
| | | Min | 33.0 | 0.17 | 41.0 | 42.6 | | 0.526 |
| | | Max | 594 | 1.00 | 1284 | 1284 | | 1.85 |
| 9 | PM | Mean | 97.6 | | 215 | 223^{b} | 602^{b} | 1.09^{b} |
| | n = 27 | % CV | 136 | | 137 | 134 | 103 | 47 |
| | | Median | 42.2 | 1.00 | 69.9 | 76.9 | 306 | 0.944 |
| | | Min | 11.0 | 0.33 | 17.3 | 18.8 | 63.7 | 0.370 |
| | | Max | 554 | 2.00 | 1076 | 1080 | 2186 | 2.79 |

^{a}n = 25;
^{b}n = 26

TABLE 10
Summary of Esomeprazole Pharmacokinetic Parameters by
Study Day and Dose Time for Treatment D (EC E20 + Naproxen)

| | Dose | Statis- | Cₘₐₓ | tₘₐₓ | AUC₀₋ₜ | AUC_{0-10, am} | AUC₀₋₂₄ | t½ |
|---|---|---|---|---|---|---|---|---|
| Day | Time | Tics | (ng/mL) | (hr) | (hr * ng/mL) | (hr * ng/mL) | (hr * ng/mL) | (hr) |
| 1 | AM | Mean | 282 | | 567 | 540^{b} | 580^{b} | 1.09^{b} |
| | n = 28 | % CV | 66 | | 68 | 60 | 67 | 44 |
| | | Median | 231 | 1.50 | 466 | 471 | 471 | 0.931 |
| | | Min | 1.1 | 1.00 | 143 | 143 | 144 | 0.633 |
| | | Max | 678 | 16.0 | 1777 | 1359 | 1777 | 2.56 |
| 9 | AM | Mean | 435 | | 1136 | 1046 | 1212^{b} | 1.27^{a} |
| | n = 28 | % CV | 48 | | 52 | 54 | 47 | 36 |
| | | Median | 453 | 1.50 | 1056 | 859 | 1123 | 1.16 |
| | | Min | 98.0 | 1.00 | 292 | 289 | 429 | 0.755 |
| | | Max | 939 | 14.0 | 2279 | 2217 | 2279 | 2.46 |

^{a}n = 25;
^{b}n = 26

Naproxen

Table 11 summarizes the results from the PK analysis that was performed for naproxen plasma concentration vs. time data for the 28 subjects who completed PN 400/E30 and EC E20+naproxen treatments and 27 subjects who completed the PN 400/E10 treatment on Days 1 and 9; and 28, and 27 subjects who completed PN 400/E20 treatment on Day 1 and Day 9, respectively.

TABLE 11
Naproxen Pharmacokinetic Parameters

| | Day/ | | | AUC_{0-10, am} or | | |
|---|---|---|---|---|---|---|
| | Dose | Cₘₐₓ | tₘₐₓ | AUC_{0-14, pm} | AUC₀₋₂₄ | t½ |
| Treatment | Time | (μg/mL) | (hr) | (hr * μg/mL) | (hr * μg/mL) | (hr) |
| A | 1 | 48.1 | 4.00 | 259 | | 8.52 |
| PN 400/E30 | AM | (53) | (2.00-10.0) | (56) | | (25) |
| | 1 | 68.9 | 14.0 | 471 | 730 | 12.1 |
| | PM | (28) | (0.50-14.0) | (30) | (32) | (30) |
| | 9 | 80.9 | 3.00 | 603 | | 9.17 |
| | AM | (23) | (0.00-8.00) | (21) | | (21) |
| | 9 | 76.2 | 10.4 | 648 | 1251 | 12.3 |
| | PM | (23) | (0.00-14.0) | (20) | (16) | (27) |
| B | 1 | 44.4 | 4.00 | 231 | | 8.75 |
| PN 400/E20 | AM | (68) | (2.00-10.0) | (70) | | (33) |
| | 1 | 71.5 | 14.0 | 450 | 680 | 11.8 |
| | PM | (26) | (0.00-14.0) | (33) | (36) | (28) |
| | 9 | 86.2 | 3.00 | 607 | | 9.42 |
| | AM | (22) | (0.00-8.05) | (19) | | (23) |
| | 9 | 76.8 | 10.0 | 678 | 1275 | 11.3 |
| | PM | (18) | (0.00-14.0) | (16) | (15) | (28) |
| C | 1 | 57.0 | 4.00 | 310 | | 9.24 |
| PN 400/E10 | AM | (31) | (2.00-10.0) | (35) | | (42) |
| | 1 | 68.6 | 10.0 | 508 | 819 | 12.7 |
| | PM | (26) | (0.00-14.0) | (29) | (21) | (23) |
| | 9 | 87.1 | 2.50 | 637 | | 9.91 |
| | AM | (21) | (0.00-8.00) | (17) | | (26) |
| | 9 | 78.6 | 14.0 | 672 | 1309 | 10.5 |
| | PM | (17) | (1.50-14.0) | (19) | (15) | (23) |
| D | 1 | 65.5 | 1.50 | 409 | | 8.85 |
| EC E20 + | AM | (25) | (0.75-6.00) | (16) | | (22) |
| naproxen | 1 | 81.5 | 1.50 | 685 | 1094 | 15.4 |
| | PM | (14) | (0.50-2.50) | (10) | (12) | (31) |
| | 9 | 90.0 | 1.50 | 617 | | 9.32 |
| | AM | (19) | (0.50-4.00) | (12) | | (23) |
| | 9 | 86.5 | 1.50 | 769 | 1387 | 14.4 |
| | PM | (13) | (0.75-4.00) | (10) | (10) | (17) |

Values are mean (% CV) for all parameters, except for tₘₐₓ, which are median (range).

Following oral administration of PN 400 on Day 1, the first measurable naproxen concentrations occurred at about 2 hrs post AM dose. Plasma exposure to naproxen was comparable among the three PN 400 treatments. Following repeated doses of PN 400, the Day 9 to Day 1 naproxen concentration ratio was consistent with the expected accumulation based on the half-life estimates of naproxen. The variability in naproxen AUC between AM and PM doses was less on Day 9 than on Day 1, reflecting that naproxen levels are approximately at steady state with repeat dosing. Cₘₐₓ values were somewhat more variable between the AM and PM doses on Day 1 compared to Day 9, with mean AM levels being lower than mean PM levels for all treatments on Day 1 and mean AM levels slightly higher than mean PM levels for all treatments on Day 9.

Following the first (AM) dose of Treatment D on Day 1, plasma naproxen concentrations were measurable in all subjects at the first sampling time, i.e., 10 minutes post-dose and then up to 24 hours post AM dose (or 14 hours post PM dose), demonstrating typical performance of non-enteric coated naproxen.

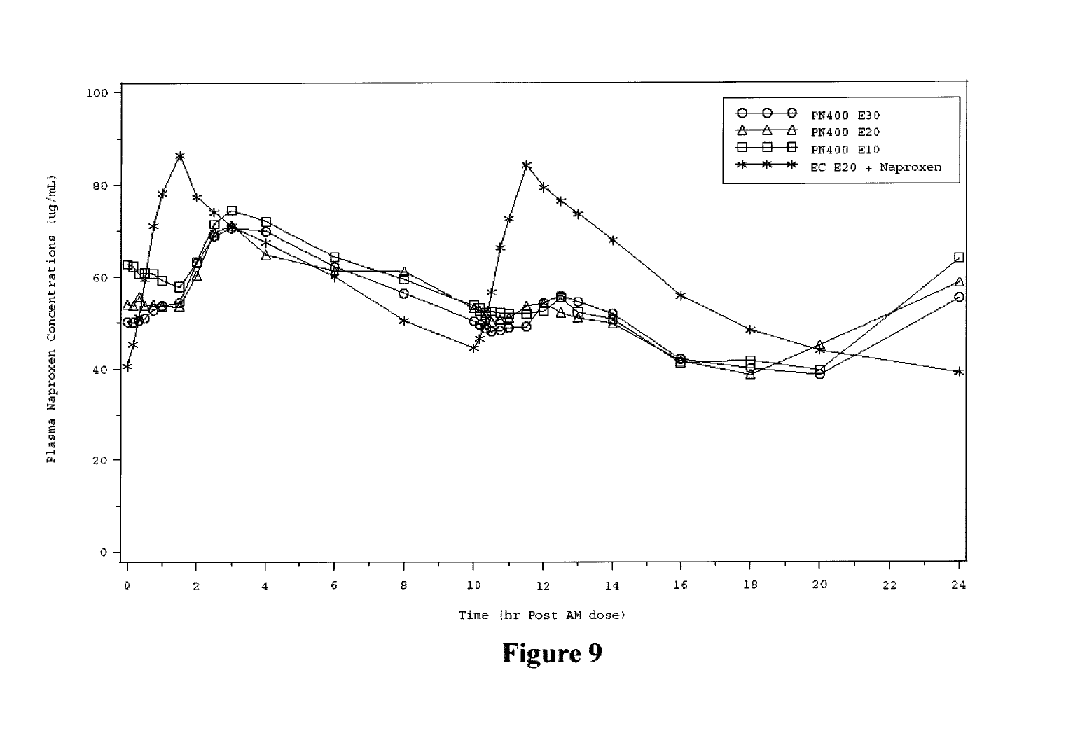

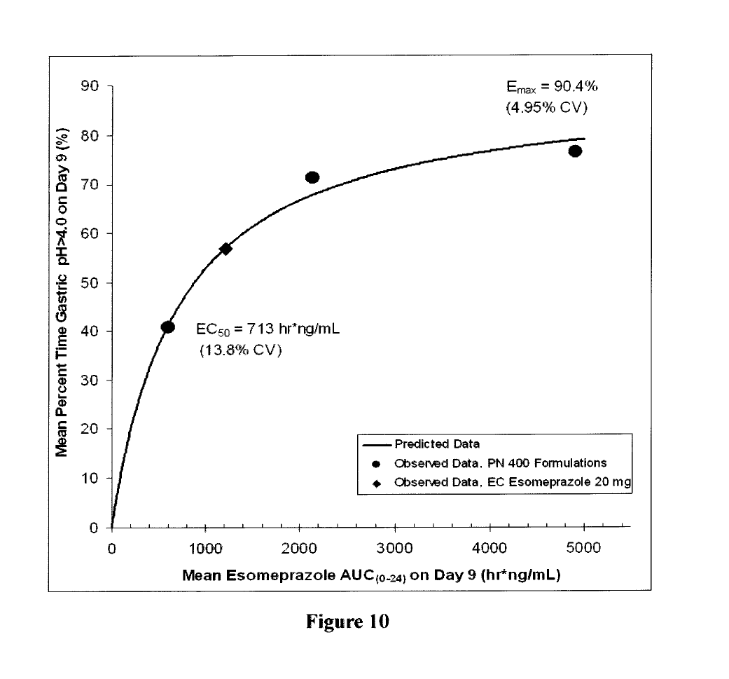

Mean and median plasma naproxen concentration vs time profiles following all 4 treatments are presented in FIGS. 8 and 9.

The PK parameters of naproxen following administration of the AM and PM doses on Day 1 and Day 9 of each treatment are summarized in Tables 12 to 15 below.

TABLE 12
Summary of Naproxen Pharmacokinetic Parameters by
Study Day and Dose Time for Treatment A (PN 400/E30)

| | | | | | | AUC_{0-10, am} or | | |
|---|---|---|---|---|---|---|---|---|
| | Dose | Statis- | Cₘₐₓ | tₘₐₓ | AUC₀₋ₜ | AUC_{0-14, pm} | AUC₀₋₂₄ | t½ |
| Day | Time | Tics | (μg/mL) | (hr) | (hr * μg/mL) | (hr * μg/mL) | (hr * μg/mL) | (hr) |
| 1 | AM | Mean | 48.1 | | 259 | 259 | | 8.52^{a} |
| | n = 28 | % CV | 53 | | 56 | 56 | | 25 |
| | | Median | 53.2 | 4.00 | 339 | 339 | | 7.86 |
| | | Min | 0.0 | 2.00 | 0.0 | 0.0 | | 6.07 |
| | | Max | 82.5 | 10.0 | 428 | 428 | | 13.8 |
| 1 | PM | Mean | 68.9 | | 471 | 471 | 730 | 12.1^{b} |
| | n = 28 | % CV | 28 | | 30 | 30 | 32 | 30 |
| | | Median | 68.5 | 14.0 | 464 | 464 | 780 | 10.8 |
| | | Min | 31.9 | 0.50 | 190 | 190 | 190 | 7.16 |
| | | Max | 114 | 14.0 | 716 | 716 | 1092 | 19.2 |
| 9 | AM | Mean | 80.9 | | 603 | 603 | | 9.17^{c} |
| | n = 28 | % CV | 23 | | 21 | 21 | | 21 |
| | | Median | 80.0 | 3.00 | 568 | 568 | | 9.31 |
| | | Min | 44.5 | 0.00 | 345 | 345 | | 5.32 |
| | | Max | 139 | 8.00 | 944 | 944 | | 13.2 |
| 9 | PM | Mean | 76.2 | | 648 | 648 | 1251 | 12.3^{d} |
| | n = 28 | % CV | 23 | | 20 | 20 | 16 | 27 |
| | | Median | 69.7 | 10.4 | 635 | 635 | 1269 | 12.0 |
| | | Min | 53.9 | 0.00 | 384 | 384 | 729 | 6.56 |
| | | Max | 127 | 14.0 | 932 | 932 | 1744 | 22.1 |

^{a}n = 17;
^{b}n = 21;
^{c}n = 26;
^{d}n = 24.

TABLE 13
Summary of Naproxen Pharmacokinetic Parameters by
Study Day and Dose Time for Treatment B (PN 400/E20)

| | | | | | | AUC_{0-10, am} or | | |
|---|---|---|---|---|---|---|---|---|
| | Dose | | Cₘₐₓ | tₘₐₓ | AUC₀₋ₜ | AUC_{0-14, pm} | AUC₀₋₂₄ | t½ |
| Day | Time | Statis-Tics | (μg/mL) | (hr) | (hr * μg/mL) | (hr * μg/mL) | (hr * μg/mL) | (hr) |
| 1 | AM | Mean | 44.4 | | 231 | 231 | | 8.75^{a} |
| | n = 28 | % CV | 68 | | 70 | 70 | | 33 |
| | | Median | 50.1 | 4.00 | 291 | 291 | | 7.72 |
| | | Min | 0.00 | 2.00 | 0.00 | 0.0 | | 6.07 |
| | | Max | 94.4 | 10.0 | 490 | 490 | | 15.1 |
| 1 | PM | Mean | 71.5 | | 450 | 450 | 680 | 11.8^{b} |
| | n = 28 | % CV | 26 | | 33 | 33 | 36 | 28 |
| | | Median | 69.4 | 14.0 | 443 | 443 | 756 | 10.9 |
| | | Min | 45.1 | 0.00 | 159 | 157 | 157 | 6.70 |
| | | Max | 110 | 14.0 | 831 | 831 | 977 | 18.7 |
| 9 | AM | Mean | 86.2 | | 607 | 607 | | 9.42^{b} |
| | n = 27 | % CV | 22 | | 19 | 19 | | 23 |
| | | Median | 85.1 | 3.00 | 577 | 577 | | 8.65 |
| | | Min | 53.5 | 0.00 | 378 | 378 | | 5.48 |
| | | Max | 137 | 8.05 | 856 | 856 | | 13.9 |
| 9 | PM | Mean | 76.8 | | 668 | 678 | 1275 | 11.3^{c} |
| | n = 27 | % CV | 18 | | 16 | 16 | 15 | 28 |
| | | Median | 73.0 | 10.0 | 661 | 661 | 1306 | 10.9 |
| | | Min | 51.2 | 0.00 | 458 | 458 | 939 | 7.97 |
| | | Max | 116 | 14.0 | 847 | 847 | 1659 | 19.2 |

^{a}n = 15,
^{b}n = 22,
^{c}n = 20

TABLE 14
Summary of Naproxen Pharmacokinetic Parameters by
Study Day and Dose Time for Treatment C (PN 400/E10)

| | | | | | | AUC_{0-10, am} or | | |
|---|---|---|---|---|---|---|---|---|
| | Dose | | Cₘₐₓ | tₘₐₓ | AUC₀₋ₜ | AUC_{0-14, pm} | AUC₀₋₂₄ | t½ |
| Day | Time | Statis-Tics | (μg/mL) | (hr) | (hr * μg/mL) | (hr * μg/mL) | (hr * μg/mL) | (hr) |
| 1 | AM | Mean | 57.0 | | 310 | 310 | | 9.24^{a} |
| | n = 27 | % CV | 31 | | 35 | 35 | | 42 |
| | | Median | 60.3 | 4.00 | 339 | 339 | | 7.88 |
| | | Min | 6.90 | 2.00 | 50.5 | 50.5 | | 3.36 |
| | | Max | 88.2 | 10.0 | 488 | 488 | | 21.8 |
| 1 | PM | Mean | 68.6 | | 508 | 508 | 819 | 12.7^{b} |
| | n = 27 | % CV | 26 | | 29 | 29 | 21 | 23 |
| | | Median | 70.0 | 10.0 | 512 | 512 | 805 | 12.5 |
| | | Min | 30.0 | 0.00 | 271 | 271 | 454 | 6.93 |
| | | Max | 97.2 | 14.0 | 791 | 791 | 1225 | 20.2 |
| 9 | AM | Mean | 87.1 | | 637 | 637 | | 9.91^{c} |
| | n = 27 | % CV | 21 | | 17 | 17 | | 26 |
| | | Median | 83.9 | 2.50 | 654 | 654 | | 9.29 |
| | | Min | 45.0 | 0.00 | 332 | 332 | | 6.88 |
| | | Max | 120 | 8.00 | 787 | 787 | | 16.1 |
| 9 | PM | Mean | 78.6 | | 672 | 672 | 1309 | 10.5^{b} |
| | n = 27 | % CV | 17 | | 19 | 19 | 15 | 23 |
| | | Median | 73.5 | 14.0 | 687 | 687 | 1328 | 9.98 |
| | | Min | 62.4 | 1.50 | 349 | 349 | 681 | 7.17 |
| | | Max | 109 | 14.0 | 953 | 953 | 1647 | 16.1 |

^{a}n = 22;
^{b}n = 25;
^{c}n = 23.

TABLE 15
Summary of Naproxen Pharmacokinetic Parameters by Study
Day and Dose Time for Treatment D (EC E20 + Naproxen)

| | | | | | | AUC_{0-10, am} or | | |
|---|---|---|---|---|---|---|---|---|
| | Dose | | Cₘₐₓ | tₘₐₓ | AUC₀₋ₜ | AUC_{0-14, pm} | AUC₀₋₂₄ | t½ |
| Day | Time | Statistics | (μg/mL) | (hr) | (hr * μg/mL) | (hr * μg/mL) | (hr * μg/mL) | (hr) |
| 1 | AM | Mean | 65.5 | | 409 | 409 | | 8.85^{a} |
| | n = 28 | % CV | 25 | | 16 | 16 | | 22 |
| | | Median | 67.0 | 1.50 | 411 | 411 | | 8.44 |
| | | Min | 39.9 | 0.75 | 293 | 293 | | 6.17 |
| | | Max | 113 | 6.00 | 562 | 562 | | 14.1 |
| 1 | PM | Mean | 81.5 | | 685 | 685 | 1094 | 15.4 |
| | n = 28 | % CV | 14 | | 10 | 10 | 12 | 31 |
| | | Median | 80.8 | 1.50 | 662 | 662 | 1068 | 14.7 |
| | | Min | 58.2 | 0.50 | 592 | 592 | 909 | 9.04 |
| | | Max | 107 | 2.50 | 855 | 855 | 1398 | 32.8 |
| 9 | AM | Mean | 90.0 | | 617 | 617 | | 9.32 |
| | n = 28 | % CV | 19 | | 12 | 12 | | 23 |
| | | Median | 87.0 | 1.50 | 619 | 619 | | 9.39 |
| | | Min | 59.4 | 0.50 | 493 | 493 | | 5.77 |
| | | Max | 126 | 4.00 | 793 | 793 | | 15.4 |
| 9 | PM | Mean | 86.5 | | 769 | 769 | 1387 | 14.4 |
| | n = 28 | % CV | 13 | | 10 | 10 | 10 | 17 |
| | | Median | 89.6 | 1.50 | 760 | 760 | 1371 | 14.7 |
| | | Min | 67.3 | 0.75 | 619 | 619 | 1130 | 10.5 |
| | | Max | 123 | 4.00 | 930 | 930 | 1723 | 21.1 |

^{a}n = 27.

### Drug Concentrations or Pharmacokinetics in Relation to Pharmacodynamic Measurements

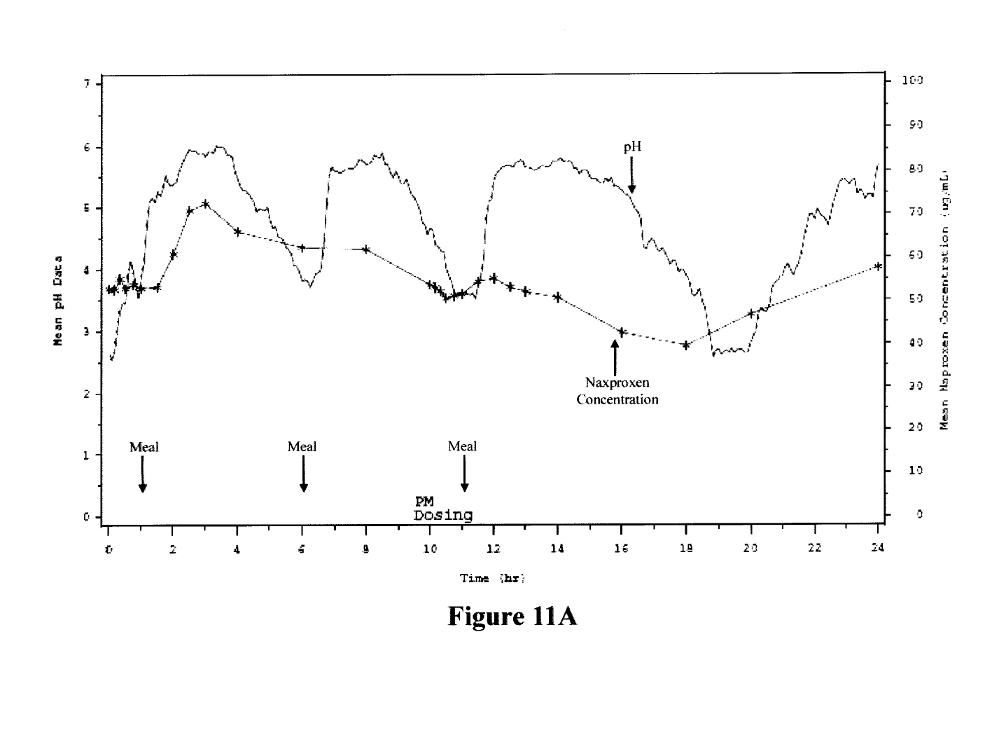

As shown in FIG. 10, the relationship between the mean total plasma exposure to esomeprazole, i.e., AUC₀₋₂₄ on Day 9 (representing steady-state exposure), and the mean percent time with intragastric pH>4.0 on Day 9 (the primary PD endpoint) can be described by a typical pharmacological maximal response (Eₘₐₓ) model defined below:

Effect=(Eₘₐₓ*AUC₀₋₂₄)/(EC50+AUC₀₋₂₄), where
Effect=Mean percent time intragastric pH>4.0 on Day 9 (assuming zero time intragastric pH>4.0 when esomeprazole AUC₀₋₂₄ equals zero)
Eₘₐₓ=Maximal Effect
EC50=Plasma mean AUC₀₋₂₄ required to produce 50% of the Maximal Effect

The Eₘₐₓ was estimated to be 90.4% of time with intragastric pH>4.0 over the daily interval at steady state. The AUC₀₋₂₄ value required to achieve half (or 50%) of the maximal response was estimated to be 713 hr*ng/mL. Following PN 400/E20, the PD response had achieved about 80% of the maximal response, which was only slightly less than that (85% of Eₘₐₓ) achieved by PN 400/E30.

Repeat doses of PN 400/E30 and PN 400/E20 resulted in faster onset of increased intragastric pH (at about 1 hour post dose) than EC E20+naproxen, which was at about 1.5 hours post-dose (FIG. 4).

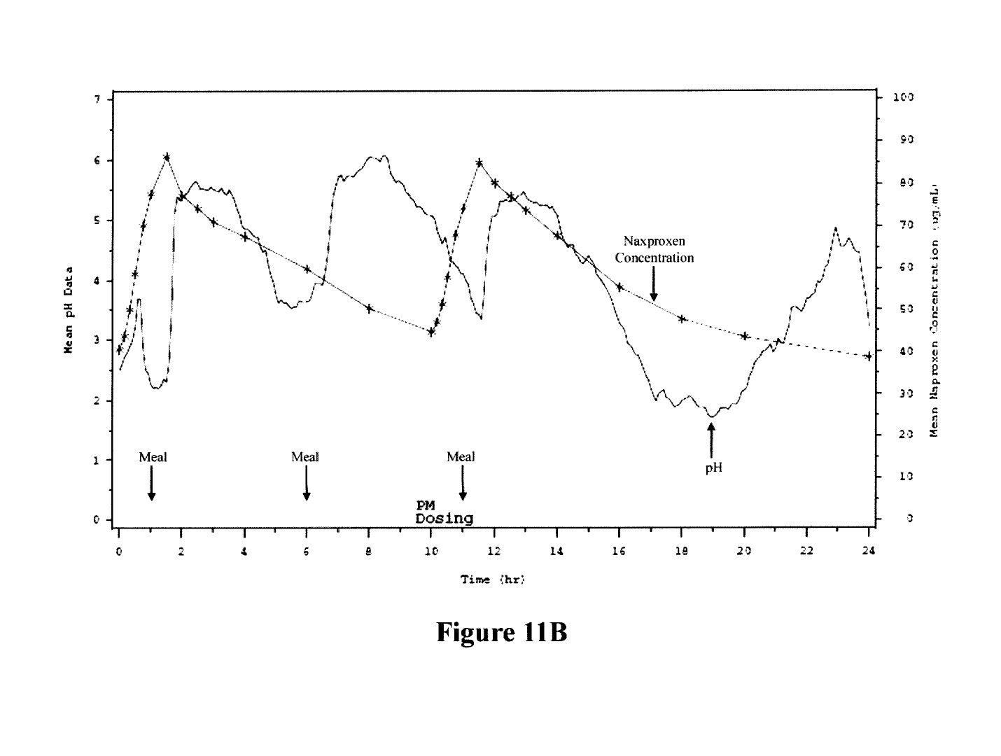

As shown in the FIG. 11A, the release of naproxen from PN 400 occurred 1.5 to 2 hours post AM dose. Before naproxen was absorbed to peak concentrations following PN 400 treatment, intragastric pH had already achieved high levels, well above pH 4.0 (FIG. 11A). In fact, with the BID regimen of PN 400/E20, given 1 hour before a meal, the intragastric pH was maintained at above 4.0 for greater than 70% of time over a 24-hour period, which would encompass any rise in plasma naproxen concentrations throughout the day.

In contrast, EC E20+naproxen produced peak naproxen concentrations that preceded the increase in intragastric pH (FIG. 11B). In fact, peak naproxen concentrations occurred 1 to 2 hours post dose, which coincided with the time period when intragastric pH was lowest (FIG. 11B).

### Example 12

### Clinical Study of the Pharmacokinetic Parameters of Naproxen and Esomeprazole

A randomized, open-label, 4-way crossover study was conducted to evaluate naproxen and esomeprazole plasma levels in healthy subjects following oral administration of PN 400, enteric-coated naproxen 500 mg plus enteric-coated esomeprazole 20 mg, enteric-coated naproxen 500 mg alone, and enteric-coated esomeprazole 20 mg alone. The study consisted of 4 single-dose treatment periods with a washout period of at least 12 days between periods. Subjects received a single dose of each of the following four treatments following a 10-hour overnight fast in a crossover fashion based on a randomization schedule with balanced treatment sequences.

| Treatment | Study Drug |
|---|---|
| A | PN 400 (delayed-release naproxen 500 mg/immediate-release |
| | esomeprazole 20 mg) tablet |
| B | EC naproxen (EC NAPROSYN ®) 500 mg tablet plus EC |
| | esomeprazole (NEXIUM ®) 20 mg capsule |
| C | EC naproxen 500 mg tablet (EC NAPROSYN ®) |
| D | EC esomeprazole 20 mg capsule (NEXIUM ®) |

EC = enteric-coated

Plasma concentration vs. time profiles of naproxen were obtained from the analysis of frequent blood samples taken over a 72-hour post-dose period following each treatment. Plasma concentration vs. time profiles of esomeprazole were obtained from the analysis of blood samples taken over a 12-hour post-dose period following Treatment A (PN 400), Treatment B (EC naproxen+EC esomeprazole), and Treatment D (EC esomeprazole alone).

Pharmacokinetic (PK) parameters of naproxen and esomeprazole were determined from the plasma concentration vs. time profiles using noncompartmental methods. PK parameters included maximum plasma concentration (Cₘₐₓ), time to maximum concentration (tₘₐₓ), lag time in absorption (t_{lag}), area under the plasma concentration vs. time curve from time zero to the last time point with measurable drug concentration (AUC₀₋ₜ), AUC from time zero with extrapolation to infinity (AUC_{0-inf}), percentage of the AUC_{0-inf} that is extrapolated from the last time point with measurable drug concentration to infinity (% AUCₑₓₜᵣₐₚ), and terminal elimination half-life (t_{1/2}).

The relative bioavailability of esomeprazole from PN 400 was determined by the ratios of AUC₀₋ₜ, AUC₀₋ₜ, and Cₘₐₓ of esomeprazole for Treatment A vs. Treatment B, and Treatment A vs. Treatment D. The relative bioavailability of naproxen from PN 400 was determined by the ratios of AUC₀₋ₜ, AUC_{0-inf}, and Cₘₐₓ of naproxen for Treatment A vs. Treatment B, and Treatment A vs. Treatment Cₘₐₓ The ratios were estimated from the analysis of variance (ANOVA) of logₑ-transformed parameters with sequence, period, and treatment as fixed effects, and subject within sequence as a random effect. The geometric least-squares mean (GLSM) ratios between treatments and the associated 90% confidence intervals for AUCs and Cₘₐₓ for each treatment comparison were calculated.

A total of forty subjects were enrolled and 37 subjects completed the study as planned, receiving all four treatments. Plasma concentration-time profiles were available from 38, 39, 39, and 39 subjects completing Treatments A, B, C, and D, respectively. Five subjects had measurable naproxen concentrations (>0.1 μg/mL) in pre-dose plasma samples during 1 or 3 dose periods. Most of the measurable pre-dose concentrations were less than 5% of the maximum concentration in each profile, and were included in the PK analysis because they would have little impact on the PK parameter estimates. However, two subjects (Subjects 1003 and 1018) had measurable pre-dose naproxen concentrations in 1 or all 3 dose periods that were >5% of the maximum concentration of the profile. In these two subjects, naproxen concentration at each time point in each profile was corrected by subtracting the residual concentration (calculated based on the decay curve from time zero (pre-dose) concentration) from the measured concentration. Subsequently, PK data analysis was based on the corrected naproxen concentrations in these two subjects.

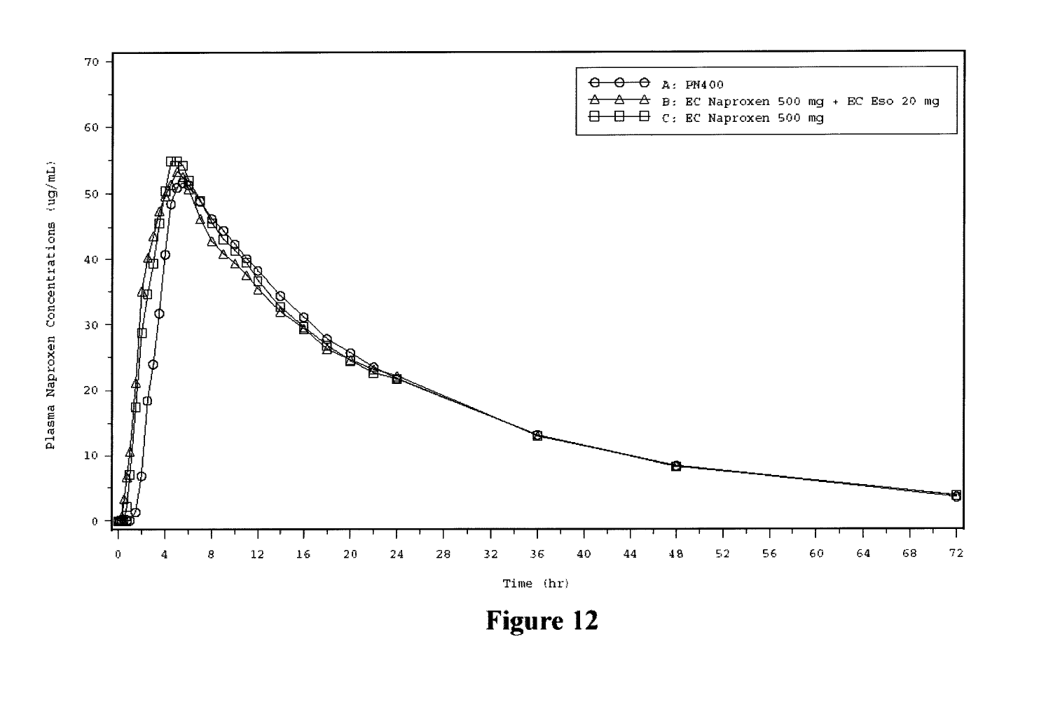

In general, plasma naproxen concentrations were not measurable at sampling times before 2 hours post dose in the majority of subjects receiving Treatment A, or before 1.5 hours post dose in the majority of subjects receiving Treatment B or Cₘₐₓ Plasma naproxen concentrations were measurable up to 72 hours post dose in all subjects after each treatment. All three treatments show very similar mean/median plasma naproxen profiles, with Treatment A (PN 400) showing slightly right-shifted (delayed) profiles (see FIG. 12).

Except in one subject in one dose period, none of the pre-dose plasma samples had quantifiable esomeprazole concentrations (all were <1 ng/mL). Following Treatment A (PN 400), plasma esomeprazole concentrations appeared rapidly in plasma, generally at the first sampling time (10 minutes post dose) in 32 out of 38 subjects. Plasma esomeprazole concentrations were measurable in all subjects at 20 minutes post dose of Treatment A. However, following Treatment B or D, plasma esomeprazole concentrations were generally not measurable until 0.75 to 1.0 hour post dose. The duration for measurable esomeprazole concentrations in plasma was about 8 hours following Treatment A, and up to 10-11 hours post dose following Treatment B or D.

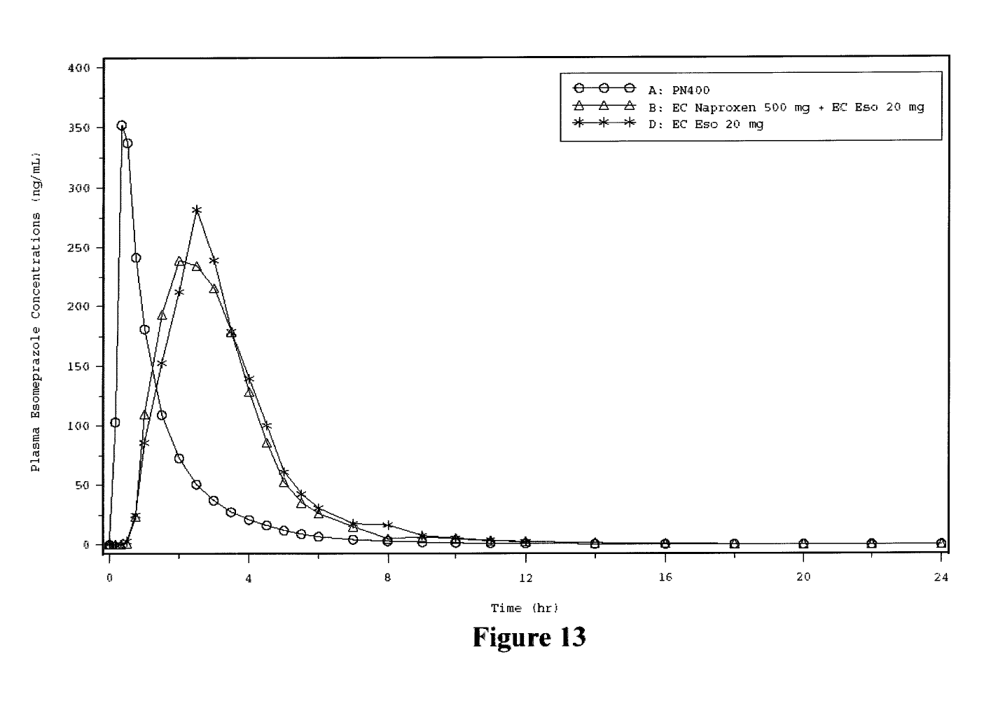

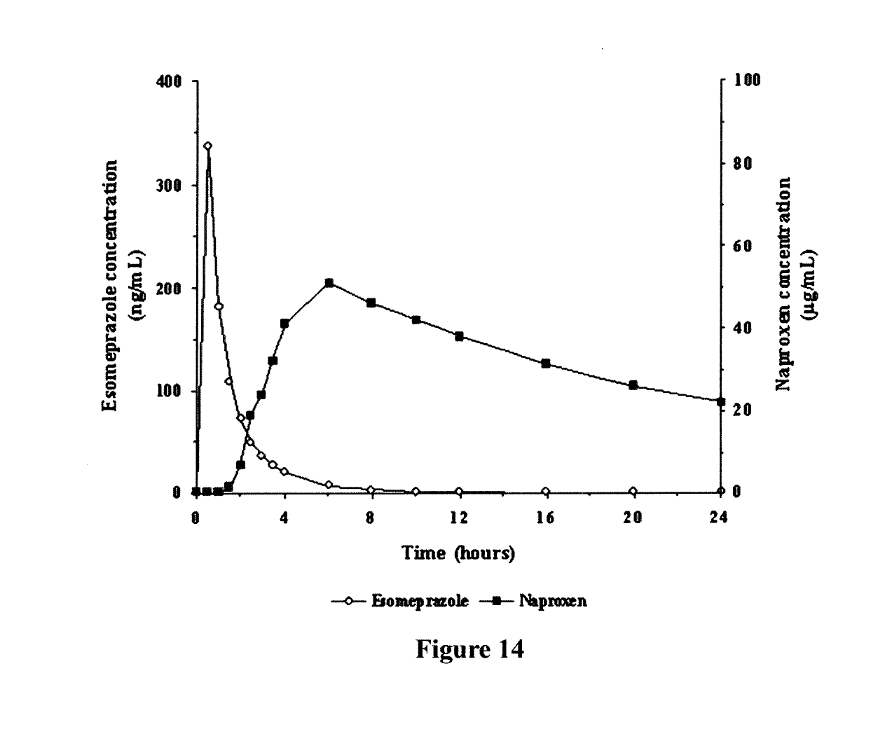

The 8-hour post-dose sample collected during Period 4 for Subject 1036 (receiving Treatment B) and Subject 1037 (receiving Treatment D) appeared to have been inadvertently switched. However, this potential sample switch could not be verified, thus PK data analysis was performed by including and excluding this time point in both subjects. Treatment A (PN 400) showed immediate-release characteristics in esomeprazole plasma profile, and Treatments B and D showed delayed-release characteristics in esomeprazole plasma profile with right-shifted curves (see FIG. 13).

A summary of naproxen and esomeprazol PK parameters by treatment are provided in the tables below.

TABLE 16
Summary of Naproxen PK Parameters by Treatment

| | | Cₘₐₓ | tₘₐₓ | t_{lag} | AUC₀₋ₜ | AUC_{0-inf} | t_{1/2} |
|---|---|---|---|---|---|---|---|
| Treatment | Statistics | (μg/mL) | (hr) | (hr) | (hr * μg/mL) | (hr * μg/mL) | (hr) |
| A | Mean | 66.9 | 6.15 | 1.98 | 1226 | 1326 | 18.9 |
| PN 400 | % CV | 22 | 58 | 53 | 15 | 17 | 14 |
| (N = 38) | Median | 66.2 | 5.25 | 1.50 | 1232 | 1324 | 19.0 |
| | Min | 37.8 | 2.50 | 0.00 | 664 | 683 | 9.20 |
| | Max | 97.0 | 20.2 | 5.00 | 1644 | 1804 | 26.8 |
| B | Mean | 74.3 | 4.95 | 1.22 | 1263 | 1374 | 19.6 |
| EC Naproxen + | % CV | 22 | 96 | 88 | 15 | 17 | 14 |
| EC Eso | Median | 74.0 | 3.50 | 1.00 | 1254 | 1359 | 18.8 |
| (N = 39) | Min | 38.7 | 0.75 | 0.00 | 908 | 985 | 13.1 |
| | Max | 114 | 24.0 | 4.00 | 1863 | 2107 | 26.1 |
| C | Mean | 75.3 | 4.99 | 1.69 | 1266 | 1375 | 19.4 |
| EC Naproxen | % CV | 20 | 85 | 69 | 15 | 16 | 11 |
| (N = 39) | Median | 77.7 | 4.00 | 1.50 | 1245 | 1345 | 19.6 |
| | Min | 35.2 | 1.50 | 0.00 | 882 | 894 | 12.4 |
| | Max | 96.0 | 24.0 | 4.50 | 1800 | 2028 | 26.2 |

Eso = esomeprazole

TABLE 17
Summary of Esomeprazole PK Parameters by Treatment

| | | Cₘₐₓ | tₘₐₓ | t_{lag} | AUC₀₋ₜ | AUC_{0-inf} | t_{1/2} |
|---|---|---|---|---|---|---|---|
| Treatment | Statistics | (ng/mL) | (hr) | (hr) | (hr * ng/mL) | (hr * ng/mL) | (hr) |
| A | Mean | 425 | 0.51 | 0.03 | 465 | 467 | 0.971 |
| PN400 | % CV | 81 | 49 | 234 | 91 | 91 | 45 |
| (N = 38) | Median | 267 | 0.45 | 0.00 | 301 | 302 | 0.881 |
| | Min | 58.1 | 0.33 | 0.00 | 104 | 107 | 0.600 |
| | Max | 1330 | 1.50 | 0.17 | 1940 | 1949 | 3.67 |
| B | Mean | 432 | 2.50 | 0.91 | 801 | 803 | 0.945 |
| EC Naproxen + | % CV | 48 | 54 | 134 | 79 | 79 | 49 |
| EC Eso | Median | 369 | 2.50 | 0.75 | 626 | 628 | 0.873 |
| (N = 39) | Min | 1.10 | 1.00 | 0.00 | 230 | 233 | 0.530 |
| | Max | 897 | 9.00 | 8.00 | 3840 | 3871 | 3.29 |
| D | Mean | 455 | 2.43 | 0.87 | 806 | 815 | 0.936 |
| EC | % CV | 40 | 34 | 47 | 78 | 81 | 44 |
| Esomeprazole | Median | 425 | 2.50 | 0.75 | 607 | 609 | 0.915 |
| (N = 39) | Min | 176 | 0.75 | 0.33 | 279 | 280 | 0.538 |
| | Max | 915 | 4.50 | 2.00 | 3707 | 3924 | 2.86 |

Eso = esomeprazole

Following oral administration of PN 400, esomeprazole was rapidly absorbed without any lag time. As expected, there was delayed absorption of esomeprazole following administration of EC esomeprazole alone or in combination with EC naproxen. Median tₘₐₓ of esomeprazole following administration of EC esomeprazole formulation was about 2 hours longer than that after administration of PN 400 containing an immediate-release formulation of esomeprazole. There was large intersubject variability in esomeprazole Cₘₐₓ and AUC values, but not t_{1/2} values for all treatments.

Statistical analysis results of bioavailability parameters of naproxen and esomeprazole for all treatment comparisons are presented in Tables 18 through 20 below.

TABLE 18
Summary of Statistical Analysis of Bioavailability Parameters
of Naproxen between Treatments (All Subjects)

| | Treatment Comparison | | |
|---|---|---|---|
| | GLSM Ratio | | |
| | (90% Confidence Interval) | | |
| PK Parameter | A/C | B/C | A/B |
| AUC_{0-inf} | 0.968 | 1.000 | 0.968 |
| (hr * μg/mL) | (0.937, 1.000) | (0.968, 1.032) | (0.938, 1.000) |
| AUC₀₋ₜ | 0.971 | 0.999 | 0.972 |
| (hr * μg/mL) | (0.942, 1.001) | (0.970, 1.029) | (0.943, 1.002) |
| Cₘₐₓ | 0.886 | 0.984 | 0.901 |
| (μg/mL) | (0.823, 0.954) | (0.915, 1.058) | (0.837, 0.970) |

GLSM = geometric least square mean

Similar results were obtained by excluding the two subjects (1003 and 1018) with pre-dose naproxen concentrations >5% of Cₘₐₓ of the profile as can be seen in Table 19.

TABLE 19
Summary of Statistical Analysis of Bioavailability Parameters
of Naproxen between Treatments (excluding Subjects 1003 and 1018))

| | Treatment Comparison | | |
|---|---|---|---|
| | GLSM Ratio | | |
| | (90% Confidence Interval) | | |
| PK Parameter | A/C | B/C | A/B |
| AUC_{0-inf} | 0.985 | 1.003 | 0.981 |
| (hr * μg/mL) | (0.960, 1.011) | (0.978, 1.029) | (0.956, 1.007) |
| AUC₀₋ₜ | 0.986 | 1.001 | 0.985 |
| (hr * μg/mL) | (0.963, 1.009) | (0.978, 1.024) | (0.962, 1.009) |
| Cₘₐₓ | 0.855 | 0.966 | 0.886 |
| (μg/mL) | (0.803, 0.911) | (0.907, 1.028) | (0.832, 0.944) |

GLSM = geometric least square mean

TABLE 20
Summary of Statistical Analysis of Bioavailability Parameters
of Esomeprazole between Treatments

| | Treatment Comparison | | |
|---|---|---|---|
| | GLSM Ratio | | |
| | (90% Confidence Interval) | | |
| PK Parameter | A/D | B/D | A/B |
| AUC_{0-inf} | 0.492 | 0.979 | 0.502 |
| (hr * ng/mL) | (0.424, 0.571) | (0.843, 1.14) | (0.432, 0.585) |
| AUC₀₋ₜ | 0.491 | 0.981 | 0.500 |
| (hr * ng/mL) | (0.422, 0.570) | (0.843, 1.14) | (0.429, 0.583) |
| Cₘₐₓ | 0.715 | 0.826 | 0.866 |
| (ng/mL) | (0.536, 0.955) | (0.620, 1.10) | (0.648, 1.16) |

GLSM = geometric least square mean

The 90% confidence interval (CI) of the geometric least squares mean (GLSM) ratio of AUC_{0-inf} and Cₘₐₓ of naproxen for all treatment comparisons fell within the 0.80 to 1.25 limits, indicating bioequivalence between treatments in terms of naproxen bioavailability parameters.

The 90% CI of the GLSM ratio (Test/Reference) of AUC_{0-inf} of esomeprazole for Treatment B vs. Treatment D fell within the 0.80 to 1.25 limits, indicating that esomeprazole AUC values for the two treatments with EC esomeprazole (with or without concomitant naproxen) are bioequivalent. Based on the point estimates and 90% CI, AUC_{0-inf} of esomeprazole for Treatment A (immediate-release esomeprazole in PN 400) were about 50% lower than those of Treatment B or D (i.e., EC esomeprazole) after a single dose administration.

An overview of aderse events (AE) is displayed in Table 21. There were no serious adverse evendts (SAEs) or withdrawals due to AEs. The incidence of all AEs ranged from 3% with Treatment C to 15% with Treatment B. The most frequent AEs included dizziness, headache, and nausea or pharyngolaryngeal pain. Most of the AEs were mild in severity.

TABLE 21
Overview of Adverse Events - Safety Population

| | Treatment | | | |
|---|---|---|---|---|
| | | B | | |
| | A | EC Nap + | C | D |
| | PN 400 | EC Eso | EC Nap | EC Eso |
| | N = 38 | N = 39 | N = 39 | N = 39 |
| | n (%) | n (%) | n (%) | n (%) |
| Subjects with at least 1 | 3 (8) | 6 (15) | 1 (3) | 2 (5) |
| adverse event | | | | |
| Subjects with at least 1 | 0 | 0 | 0 | 0 |
| serious adverse vent | | | | |
| Deaths | 0 | 0 | 0 | 0 |
| Withdrawals due to adverse | 0 | 0 | 0 | 0 |
| events | | | | |

EC = enteric-coated;
Nap = naproxen;
Eso = esomeprazole
A = PN 400 = naproxen 500 mg/esomeprazole 20 mg
B = EC Nap + EC Eso = enteric-coated naproxen 500 mg + enteric-coated esomeprazole 20 mg
C = EC Nap = enteric-coated naproxen 500 mg
D = EC Eso = enteric-coated esomeprazole 20 mg

AE data are summarized by system organ class (SOC) and provided in Table 22. The overall occurrence rate was notably greater with Treatment B (15%) than with the other treatments (3-8%). The only AEs occurring in more than 1 subject per treatment period were dizziness, which occurred in 2 subjects (5%) with Treatment B; headache, which occurred in 2 subjects with Treatment B; and pharyngolaryngeal pain, which occurred in 3 subjects (8%) with Treatment B.

TABLE 22
Treatment-Emergent Adverse Events - Safety Population

| | | B | | |
|---|---|---|---|---|
| | A | EC Nap + | C | D |
| | PN 400 | EC Eso | EC Nap | EC Eso |
| System Organ Class | N = 38 | N = 39 | N = 39 | N = 39 |
| Adverse Event | n (%) | n (%) | n (%) | n (%) |
| Subjects with at least 1 adverse | 3 (8) | 6 (15) | 1 (3) | 2 (5) |
| event | | | | |
| Nervous system disorders | 2 (5) | 3 (8) | 0 | 2 (5) |
| Dizziness | 1 (3) | 2 (5) | 0 | 1 (3) |
| Headache | 0 | 2 (5) | 0 | 1 (3) |
| Syncope | 1 (3) | 0 | 0 | 0 |
| General disorders and | 1 (3) | 1 (3) | 1 (3) | 0 |
| administration site conditions | | | | |
| Chest discomfort | 0 | 0 | 1 (3) | 0 |
| Cyst | 1 (3) | 0 | 0 | 0 |
| Pain | 0 | 1 (3) | 0 | 0 |
| Pyrexia | 0 | 1 (3) | 0 | 0 |
| Respiratory, thoracic and | 0 | 3 (8) | 0 | 0 |
| mediastinal disorders | | | | |
| Pharyngolaryngeal pain | 0 | 3 (8) | 0 | 0 |
| Cough | 0 | 1 (3) | 0 | 0 |
| Gastrointestinal disorders | 0 | 1 (3) | 0 | 0 |
| Nausea | 0 | 1 (3) | 0 | 0 |
| Injury, poisoning and procedural | 1 (3) | 0 | 0 | 0 |
| complications | | | | |
| Head Injury | 1 (3) | 0 | 0 | 0 |

EC = enteric-coated;
Nap = naproxen;
Eso = esomeprazole
A = PN 400 = naproxen 500 mg/esomeprazole 20 mg
B = EC Nap + EC Eso = enteric-coated naproxen 500 mg + enteric-coated esomeprazole 20 mg
C = EC Nap = enteric-coated naproxen 500 mg
D = EC Eso = enteric-coated esomeprazole 20

The only AEs assessed as treatment-related by the investigator were nausea, dizziness and headache in one subject and headache and pyrexia in another subject, each occurring during Treatment B.

All references cited herein are fully incorporated by reference. Having now fully described the invention, it will be understood by those of skill in the art that the invention may be performed within a wide and equivalent range of conditions, parameters and the like, without affecting the spirit or scope of the invention or any embodiment thereof.

1.-56. (canceled)

57. A pharmaceutical composition in unit dosage form suitable for administration to a patient, comprising:
(a) esomeprazole, at least a portion of which is not surrounded by an enteric coating; and
(b) naproxen, wherein the naproxen is surrounded by a coating that substantially inhibits the release of the naproxen from the dosage form unless it is exposed to an environment with a pH of about 3.5 or higher;
wherein the unit dosage form provides for release of the esomeprazole and the naproxen such that:
i) upon introduction of the unit dosage form into a medium, at least a portion of the esomeprazole is released regardless of the pH of the medium; and
ii) the naproxen is released when the pH of the environment is 3.5 or higher.

58. The pharmaceutical composition of claim 57, wherein the coating surrounding the naproxen dissolves when the pH of the environment is 5.5 or higher.

59. The pharmaceutical composition of claim 57, wherein the naproxen is present in an amount of between 200 mg and 600 mg.

60. The pharmaceutical composition of claim 57, wherein the esomeprazole is present in an amount of between 5 mg and 100 mg.

61. The pharmaceutical composition of claim 57, wherein the unit dosage form is suitable for oral administration to a patient.

62. The pharmaceutical composition of claim 57, wherein the unit dosage form is a tablet.

63. The pharmaceutical composition of claim 57, wherein the unit dosage form is a multilayer tablet comprising a single core and one or more layers outside of the core, wherein:
i) the naproxen is present in the core;
ii) the coating surrounds the core; and
iii) the esomeprazole is in the one or more layers outside the core.

64. The pharmaceutical composition of claim 63, wherein the one or more layers outside of the core do not contain naproxen and are not surrounded by an enteric coating.

65. The pharmaceutical composition of claim 63, wherein the unit dosage form is a bilayer tablet having an outer layer of the esomeprazole and an inner core of the naproxen and wherein the outer layer of the tablet is surrounded by a non-enteric film coating.

66. The pharmaceutical composition of claim 63, wherein the coating surrounding the core substantially inhibits the release of the naproxen unless it is in a medium with a pH of 5.5 or greater.

67. A method of treating pain or inflammation in a patient in need of such treatment, comprising:
(a) orally administering to the patient esomeprazole, wherein at least a portion of the esomeprazole is not surrounded by an enteric coating, wherein the dose of the esomeprazole is effective to raise the gastric pH of the patient to at least 3.5 or higher; and
(b) orally administering to the patient naproxen, wherein the naproxen is surrounded by a coating that substantially inhibits the release of the naproxen unless it is in an environment with a pH of 3.5 or higher,
wherein the naproxen is released and is effective to treat the pain or inflammation of the patient.

68. The method of claim 67, wherein the coating surrounding the naproxen substantially inhibits the release of the naproxen unless it is in an environment with a pH of 5.5 or higher.

69. The method of claim 67, wherein the pain or inflammation is due to osteoarthritis or rheumatoid arthritis.

70. The method of claim 67, wherein the naproxen is administered in an amount of between 200 mg and 600 mg.

71. The method of claim 67, wherein the esomeprazole is administered in an amount of between 5 mg and 100 mg.

72. The method of claim 67, wherein the esomeprazole and the naproxen are delivered as a unit dosage form.

73. The method of claim 72, wherein the unit dosage form provides for the sequential release of the esomeprazole followed by the naproxen.

74. The method of claim 72, wherein the unit dosage form is a tablet.

75. The method of claim 72, wherein the unit dosage form is a bilayer tablet having an outer layer of the esomeprazole and an inner core of the naproxen and wherein the outer layer of the tablet is surrounded by a non-enteric film coating.

76. A method of treating ankylosing spondylitis in a patient in need of such treatment, comprising:
(a) orally administering to the patient esomeprazole, wherein at least a portion of the esomeprazole is not surrounded by an enteric coating, wherein the dose of the esomeprazole is effective to raise the gastric pH of the patient to at least 3.5 or higher; and
(b) orally administering to the patient naproxen, wherein the naproxen is surrounded by a coating that substantially inhibits the release of the naproxen unless it is in an environment with a pH of 3.5 or higher,
wherein the naproxen is released and is effective to treat the ankylosing spondylitis of the patient.
